SEQUENCE LISTING (328 sequences)

SEQ ID NO: 1 - DNA, 1431 bp, Synthetic construct
  source 1..1431: mol_type = other DNA
agccctccaggacaggctgcatcagaagaggccatcaagcaggtctgttccaagggcctttgcgtcaggtgggctcaggattccagggtggctggaccccaggccccagctctgcagcagggaggacgtggctgggctcgtgaagcatgtgggggtgagcccaggggccccaaggcagggcacctggccttcagcctgcctcagccctgcctgtctcccagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcagggtgagccaactgcccattgctgcccctggccgcccccagccaccccctgctcctggcgctcccacccagcatgggcagaagggggcaggaggctgccacccagcagggggtcaggtgcacttttttaaaaagaagttctcttggtcacgtcctaaaagtgaccagctccctgtggcccagtcagaatctcagcctgaggacggtgttggcttcggcagccccgagatacatcagagggtgggcacgctcctccctccactcgcccctcaaacaaatgccccgcagcccatttctccaccctcatttgatgaccgcagattcaagtgttttgttaagtaaagtcctgggtgacctggggtcacagggtgccccacgctgcctgcctctgggcgaacaccccatcacgcccggaggagggcgtggctgcctgcctgagtgggccagacccctgtcgccaggcctcacggcagctccatagtcaggagatggggaagatgctggggacaggccctggggagaagtactgggatcacctgttcaggctcccactgtgacgctgccccggggcgggggaaggaggtgggacatgtgggcgttggggcctgtaggtccacacccagtgtgggtgaccctccctctaacctgggtccagcccggctggagatgggtgggagtgcgacctagggctggcgggcaggcgggcactgtgtctccctgactgtgtcctcctgtgtccctctgcctcgccgctgttccggaacctgctctgcgcggcacgtcctggcagtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagc

SEQ ID NO: 2 - DNA, 50 bp, Synthetic construct
  source 1..50: mol_type = other DNA
gcaccaaacgaaaatactaagggccaacgataccgggtgtacggacacag

SEQ ID NO: 3 - DNA, 50 bp, Synthetic construct
  source 1..50: mol_type = other DNA
tcatctgtgtccgtacacccggtatcgttggcccttagtattttcgtttg

SEQ ID NO: 4 - DNA, 50 bp, Synthetic construct
  source 1..50: mol_type = other DNA
atgattacgtctaaatctcatgcccagtagaaacaggcgcggccagaaag

SEQ ID NO: 5 - DNA, 50 bp, Synthetic construct
  source 1..50: mol_type = other DNA
caatctttctggccgcgcctgtttctactgggcatgagatttagacgtaa

SEQ ID NO: 6 - DNA, 50 bp, Synthetic construct
  source 1..50: mol_type = other DNA
attgaggcgatcgcccacccgaatggagtcacaagccacaacccgtattg

SEQ ID NO: 7 - DNA, 50 bp, Synthetic construct
  source 1..50: mol_type = other DNA
caatcaatacgggttgtggcttgtgactccattcgggtgggcgatcgcct

SEQ ID NO: 8 - DNA, 50 bp, Synthetic construct
  source 1..50: mol_type = other DNA
attgtctattgcaattgattgccgcatcatctagtgatgaggacacctga

SEQ ID NO: 9 - DNA, 50 bp, Synthetic construct
  source 1..50: mol_type = other DNA
gtgttcaggtgtcctcatcactagatgatgcggcaatcaattgcaataga

SEQ ID NO: 10 - DNA, 50 bp, Synthetic construct
  source 1..50: mol_type = other DNA
acaccttactccctaattcgtcaccacattgttgtgagccatagggggac

SEQ ID NO: 11 - DNA, 50 bp, Synthetic construct
  source 1..50: mol_type = other DNA
ggatgtccccctatggctcacaacaatgtggtgacgaattagggagtaag

SEQ ID NO: 12 - DNA, 50 bp, Synthetic construct
  source 1..50: mol_type = other DNA
atcctgtttaagctagttagttagcacgtgaagacgtgggttcgggttag

SEQ ID NO: 13 - DNA, 50 bp, Synthetic construct
  source 1..50: mol_type = other DNA
acggctaacccgaacccacgtcttcacgtgctaactaactagcttaaaca

SEQ ID NO: 14 - DNA, 50 bp, Synthetic construct
  source 1..50: mol_type = other DNA
ccgtccaaccatctgaagggggaattgtaaacgtcggtagggcctggcac

SEQ ID NO: 15 - DNA, 50 bp, Synthetic construct
  source 1..50: mol_type = other DNA
ggaggtgccaggccctaccgacgtttacaattcccccttcagatggttgg

SEQ ID NO: 16 - DNA, 50 bp, Synthetic construct
  source 1..50: mol_type = other DNA
ctcctggcgctctcggttatcggtgctgcgcaaacccggagcagaggcgt

SEQ ID NO: 17 - DNA, 50 bp, Synthetic construct
  source 1..50: mol_type = other DNA
gcttacgcctctgctccgggtttgcgcagcaccgataaccgagagcgcca

SEQ ID NO: 18 - DNA, 50 bp, Synthetic construct
  source 1..50: mol_type = other DNA
aagcgtggtccgacgtgtgagcgtgcccgttagcgagtggggtcagggtg

SEQ ID NO: 19 - DNA, 50 bp, Synthetic construct
  source 1..50: mol_type = other DNA
tccgcaccctgaccccactcgctaacgggcacgctcacacgtcggaccac

SEQ ID NO: 20 - DNA, 50 bp, Synthetic construct
  source 1..50: mol_type = other DNA
cggagtgaacaccacccccattggtctaatgccctatcaaagagagccca

SEQ ID NO: 21 - DNA, 50 bp, Synthetic construct
  source 1..50: mol_type = other DNA
gcattgggctctctttgatagggcattagaccaatgggggtggtgttcac

SEQ ID NO: 22 - DNA, 50 bp, Synthetic construct
  source 1..50: mol_type = other DNA
atgcccttcgataccgaatatccatctcggtcgaacccatattgcgataa

SEQ ID NO: 23 - DNA, 50 bp, Synthetic construct
  source 1..50: mol_type = other DNA
aaccttatcgcaatatgggttcgaccgagatggatattcggtatcgaagg

SEQ ID NO: 24 - DNA, 50 bp, Synthetic construct
  source 1..50: mol_type = other DNA
ggttctctggcggcaagaccctcagtcgataactcccggctaggggtatg

SEQ ID NO: 25 - DNA, 50 bp, Synthetic construct
  source 1..50: mol_type = other DNA
tactcatacccctagccgggagttatcgactgagggtcttgccgccagag

SEQ ID NO: 26 - DNA, 128 bp, Synthetic construct
  source 1..128: mol_type = other DNA
tgtagtcatgcccacgggtgcaaagcacgaaacagcagcggaggagaagtatcgtacgggggtcaggtgactataggttatcactggtagggtaccgcccatagtccccatgatacccgattcggtct

SEQ ID NO: 27 - DNA, 16 bp, Synthetic construct
  source 1..16: mol_type = other DNA
tgtagtcatgcccacg

SEQ ID NO: 28 - DNA, 16 bp, Synthetic construct
  source 1..16: mol_type = other DNA
cacccgtgggcatgac

SEQ ID NO: 29 - DNA, 16 bp, Synthetic construct
  source 1..16: mol_type = other DNA
ggtgcaaagcacgaaa

SEQ ID NO: 30 - DNA, 16 bp, Synthetic construct
  source 1..16: mol_type = other DNA
gctgtttcgtgctttg

SEQ ID NO: 31 - DNA, 16 bp, Synthetic construct
  source 1..16: mol_type = other DNA
cagcagcggaggagaa

SEQ ID NO: 32 - DNA, 16 bp, Synthetic construct
  source 1..16: mol_type = other DNA
atacttctcctccgct

SEQ ID NO: 33 - DNA, 16 bp, Synthetic construct
  source 1..16: mol_type = other DNA
gtatcgtacgggggtc

SEQ ID NO: 34 - DNA, 16 bp, Synthetic construct
  source 1..16: mol_type = other DNA
acctgacccccgtacg

SEQ ID NO: 35 - DNA, 16 bp, Synthetic construct
  source 1..16: mol_type = other DNA
aggtgactataggtta

SEQ ID NO: 36 - DNA, 16 bp, Synthetic construct
  source 1..16: mol_type = other DNA
gtgataacctatagtc

SEQ ID NO: 37 - DNA, 16 bp, Synthetic construct
  source 1..16: mol_type = other DNA
tcactggtagggtacc

SEQ ID NO: 38 - DNA, 16 bp, Synthetic construct
  source 1..16: mol_type = other DNA
gggcggtaccctacca

SEQ ID NO: 39 - DNA, 16 bp, Synthetic construct
  source 1..16: mol_type = other DNA
gcccatagtccccatg

SEQ ID NO: 40 - DNA, 16 bp, Synthetic construct
  source 1..16: mol_type = other DNA
gtatcatggggactat

SEQ ID NO: 41 - DNA, 16 bp, Synthetic construct
  source 1..16: mol_type = other DNA
atacccgattcggtct

SEQ ID NO: 42 - DNA, 16 bp, Synthetic construct
  source 1..16: mol_type = other DNA
tggtagaccgaatcgg

SEQ ID NO: 43 - DNA, 260 bp, Synthetic construct
  source 1..260: mol_type = other DNA
gcaccaaacgaaaatactatgggccaacgataccgagtgtacggacacagacgattacgtctgaatctcatgcccagtagtcggaggcgcggccagagatattgaggcgatcagccacccgaatggagaacaaagccacaacccgtattgcgtctctatttgttttgattgccgcatcatctagtgatgagggcacctgaacaccttaatacctaattcgtcaccacattgttgtgagccatagggggacatcctgttta

SEQ ID NO: 44 - DNA, 16 bp, Synthetic construct
  source 1..16: mol_type = other DNA
gcaccaaacgaaaata

SEQ ID NO: 45 - DNA, 16 bp, Synthetic construct
  source 1..16: mol_type = other DNA
atagtattttcgtttg

SEQ ID NO: 46 - DNA, 16 bp, Synthetic construct
  source 1..16: mol_type = other DNA
ctatgggccaacgata

SEQ ID NO: 47 - DNA, 16 bp, Synthetic construct
  source 1..16: mol_type = other DNA
tcggtatcgttggccc

SEQ ID NO: 48 - DNA, 16 bp, Synthetic construct
  source 1..16: mol_type = other DNA
ccgagtgtacggacac

SEQ ID NO: 49 - DNA, 16 bp, Synthetic construct
  source 1..16: mol_type = other DNA
gtctgtgtccgtacac

SEQ ID NO: 50 - DNA, 16 bp, Synthetic construct
  source 1..16: mol_type = other DNA
agacgattacgtctga

SEQ ID NO: 51 - DNA, 16 bp, Synthetic construct
  source 1..16: mol_type = other DNA
agattcagacgtaatc

SEQ ID NO: 52 - DNA, 16 bp, Synthetic construct
  source 1..16: mol_type = other DNA
atctcatgcccagtag

SEQ ID NO: 53 - DNA, 16 bp, Synthetic construct
  source 1..16: mol_type = other DNA
ccgactactgggcatg

SEQ ID NO: 54 - DNA, 16 bp, Synthetic construct
  source 1..16: mol_type = other DNA
tcggaggcgcggccag

SEQ ID NO: 55 - DNA, 16 bp, Synthetic construct
  source 1..16: mol_type = other DNA
atctctggccgcgcct

SEQ ID NO: 56 - DNA, 16 bp, Synthetic construct
  source 1..16: mol_type = other DNA
agatattgaggcgatc

SEQ ID NO: 57 - DNA, 16 bp, Synthetic construct
  source 1..16: mol_type = other DNA
ggctgatcgcctcaat

SEQ ID NO: 58 - DNA, 16 bp, Synthetic construct
  source 1..16: mol_type = other DNA
agccacccgaatggag

SEQ ID NO: 59 - DNA, 16 bp, Synthetic construct
  source 1..16: mol_type = other DNA
tgttctccattcgggt

SEQ ID NO: 60 - DNA, 16 bp, Synthetic construct
  source 1..16: mol_type = other DNA
aacaaagccacaaccc

SEQ ID NO: 61 - DNA, 16 bp, Synthetic construct
  source 1..16: mol_type = other DNA
atacgggttgtggctt

SEQ ID NO: 62 - DNA, 16 bp, Synthetic construct
  source 1..16: mol_type = other DNA
gtattgcgtctctatt

SEQ ID NO: 63 - DNA, 16 bp, Synthetic construct
  source 1..16: mol_type = other DNA
aacaaatagagacgca

SEQ ID NO: 64 - DNA, 16 bp, Synthetic construct
  source 1..16: mol_type = other DNA
tgttttgattgccgca

SEQ ID NO: 65 - DNA, 16 bp, Synthetic construct
  source 1..16: mol_type = other DNA
atgatgcggcaatcaa

SEQ ID NO: 66 - DNA, 16 bp, Synthetic construct
  source 1..16: mol_type = other DNA
tcatctagtgatgagg

SEQ ID NO: 67 - DNA, 16 bp, Synthetic construct
  source 1..16: mol_type = other DNA
gtgccctcatcactag

SEQ ID NO: 68 - DNA, 16 bp, Synthetic construct
  source 1..16: mol_type = other DNA
gcacctgaacacctta

SEQ ID NO: 69 - DNA, 16 bp, Synthetic construct
  source 1..16: mol_type = other DNA
gtattaaggtgttcag

SEQ ID NO: 70 - DNA, 16 bp, Synthetic construct
  source 1..16: mol_type = other DNA
atacctaattcgtcac

SEQ ID NO: 71 - DNA, 16 bp, Synthetic construct
  source 1..16: mol_type = other DNA
tgtggtgacgaattag

SEQ ID NO: 72 - DNA, 16 bp, Synthetic construct
  source 1..16: mol_type = other DNA
cacattgttgtgagcc

SEQ ID NO: 73 - DNA, 16 bp, Synthetic construct
  source 1..16: mol_type = other DNA
ctatggctcacaacaa

SEQ ID NO: 74 - DNA, 16 bp, Synthetic construct
  source 1..16: mol_type = other DNA
atagggggacatcctg

SEQ ID NO: 75 - DNA, 16 bp, Synthetic construct
  source 1..16: mol_type = other DNA
cacttagtgctcgtcc

SEQ ID NO: 76 - DNA, 16 bp, Synthetic construct
  source 1..16: mol_type = other DNA
cgttgatcgctggtgc

SEQ ID NO: 77 - DNA, 16 bp, Synthetic construct
  source 1..16: mol_type = other DNA
atcagggccttgagct

SEQ ID NO: 78 - DNA, 16 bp, Synthetic construct
  source 1..16: mol_type = other DNA
ggcctgttggtgggcc

SEQ ID NO: 79 - DNA, 16 bp, Synthetic construct
  source 1..16: mol_type = other DNA
tgctaacgccgccctc

SEQ ID NO: 80 - DNA, 16 bp, Synthetic construct
  source 1..16: mol_type = other DNA
ctgtggacgaagtgga

SEQ ID NO: 81 - DNA, 16 bp, Synthetic construct
  source 1..16: mol_type = other DNA
atgagagacgtgcgcc

SEQ ID NO: 82 - DNA, 16 bp, Synthetic construct
  source 1..16: mol_type = other DNA
atgttatctccgccag

SEQ ID NO: 83 - DNA, 16 bp, Synthetic construct
  source 1..16: mol_type = other DNA
ccgtccatgcgtgacc

SEQ ID NO: 84 - DNA, 16 bp, Synthetic construct
  source 1..16: mol_type = other DNA
gggtcgggtacgaccg

SEQ ID NO: 85 - DNA, 16 bp, Synthetic construct
  source 1..16: mol_type = other DNA
ggccggaacggactca

SEQ ID NO: 86 - DNA, 16 bp, Synthetic construct
  source 1..16: mol_type = other DNA
cacccatggtgagcaa

SEQ ID NO: 87 - DNA, 16 bp, Synthetic construct
  source 1..16: mol_type = other DNA
tacgtatccaccggga

SEQ ID NO: 88 - DNA, 16 bp, Synthetic construct
  source 1..16: mol_type = other DNA
gcttagtttggccccc

SEQ ID NO: 89 - DNA, 16 bp, Synthetic construct
  source 1..16: mol_type = other DNA
ttgggccgacccgggg

SEQ ID NO: 90 - DNA, 16 bp, Synthetic construct
  source 1..16: mol_type = other DNA
agtccgaacgctcggg

SEQ ID NO: 91 - DNA, 16 bp, Synthetic construct
  source 1..16: mol_type = other DNA
actactacttcacgag

SEQ ID NO: 92 - DNA, 16 bp, Synthetic construct
  source 1..16: mol_type = other DNA
caagcatctccggcgc

SEQ ID NO: 93 - DNA, 16 bp, Synthetic construct
  source 1..16: mol_type = other DNA
cgctgcccgcgatctg

SEQ ID NO: 94 - DNA, 16 bp, Synthetic construct
  source 1..16: mol_type = other DNA
gcacgccccttccaga

SEQ ID NO: 95 - DNA, 16 bp, Synthetic construct
  source 1..16: mol_type = other DNA
ctcctgggcagcgaca

SEQ ID NO: 96 - DNA, 16 bp, Synthetic construct
  source 1..16: mol_type = other DNA
gcgccgagagggcggt

SEQ ID NO: 97 - DNA, 16 bp, Synthetic construct
  source 1..16: mol_type = other DNA
cgcccaggggaaccgc

SEQ ID NO: 98 - DNA, 16 bp, Synthetic construct
  source 1..16: mol_type = other DNA
cctacagcccccgaga

SEQ ID NO: 99 - DNA, 16 bp, Synthetic construct
  source 1..16: mol_type = other DNA
tcgtttctccccccgc

SEQ ID NO: 100 - DNA, 16 bp, Synthetic construct
  source 1..16: mol_type = other DNA
ttccacccctgggcga

SEQ ID NO: 101 - DNA, 16 bp, Synthetic construct
  source 1..16: mol_type = other DNA
ggtgcatacctttcaa

SEQ ID NO: 102 - DNA, 16 bp, Synthetic construct
  source 1..16: mol_type = other DNA
cccgtcaaatgccact

SEQ ID NO: 103 - DNA, 16 bp, Synthetic construct
  source 1..16: mol_type = other DNA
tagaacgtcctgctcg

SEQ ID NO: 104 - DNA, 16 bp, Synthetic construct
  source 1..16: mol_type = other DNA
tccgtagaaaacaggc

SEQ ID NO: 105 - DNA, 16 bp, Synthetic construct
  source 1..16: mol_type = other DNA
agacgccggacgccca

SEQ ID NO: 106 - DNA, 16 bp, Synthetic construct
  source 1..16: mol_type = other DNA
tatgggaggcgctcgt

SEQ ID NO: 107 - DNA, 16 bp, Synthetic construct
  source 1..16: mol_type = other DNA
tggcccgctgggcgac

SEQ ID NO: 108 - DNA, 16 bp, Synthetic construct
  source 1..16: mol_type = other DNA
tgggcgtgactggcga

SEQ ID NO: 109 - DNA, 16 bp, Synthetic construct
  source 1..16: mol_type = other DNA
ccgccaggtatccccg

SEQ ID NO: 110 - DNA, 16 bp, Synthetic construct
  source 1..16: mol_type = other DNA
gggggagcgcgccacg

SEQ ID NO: 111 - DNA, 16 bp, Synthetic construct
  source 1..16: mol_type = other DNA
ctgcaggcggacgcga

SEQ ID NO: 112 - DNA, 16 bp, Synthetic construct
  source 1..16: mol_type = other DNA
gggagatgcagggacg

SEQ ID NO: 113 - DNA, 16 bp, Synthetic construct
  source 1..16: mol_type = other DNA
gggtggtcagcttgac

SEQ ID NO: 114 - DNA, 16 bp, Synthetic construct
  source 1..16: mol_type = other DNA
ccgggcctgggaggct

SEQ ID NO: 115 - DNA, 16 bp, Synthetic construct
  source 1..16: mol_type = other DNA
cgtccggcccctcgcg

SEQ ID NO: 116 - DNA, 16 bp, Synthetic construct
  source 1..16: mol_type = other DNA
gctagactgggatagc

SEQ ID NO: 117 - DNA, 16 bp, Synthetic construct
  source 1..16: mol_type = other DNA
ggtataccagcgggag

SEQ ID NO: 118 - DNA, 16 bp, Synthetic construct
  source 1..16: mol_type = other DNA
gagtctctccggcgtc

SEQ ID NO: 119 - DNA, 16 bp, Synthetic construct
  source 1..16: mol_type = other DNA
cggccctgcaggcaaa

SEQ ID NO: 120 - DNA, 16 bp, Synthetic construct
  source 1..16: mol_type = other DNA
ggcgtgagcgtttccc

SEQ ID NO: 121 - DNA, 16 bp, Synthetic construct
  source 1..16: mol_type = other DNA
gctgtccgggcggtag

SEQ ID NO: 122 - DNA, 16 bp, Synthetic construct
  source 1..16: mol_type = other DNA
cggctggggccatgtc

SEQ ID NO: 123 - DNA, 16 bp, Synthetic construct
  source 1..16: mol_type = other DNA
cgacggcgcgcgatgt

SEQ ID NO: 124 - DNA, 16 bp, Synthetic construct
  source 1..16: mol_type = other DNA
agtcggggcccattcc

SEQ ID NO: 125 - DNA, 16 bp, Synthetic construct
  source 1..16: mol_type = other DNA
acggtgcaacgtcccc

SEQ ID NO: 126 - DNA, 16 bp, Synthetic construct
  source 1..16: mol_type = other DNA
ctacagtaggtcaatc

SEQ ID NO: 127 - DNA, 16 bp, Synthetic construct
  source 1..16: mol_type = other DNA
ggcaggcacaggcgcg

SEQ ID NO: 128 - DNA, 16 bp, Synthetic construct
  source 1..16: mol_type = other DNA
cgcgcccgccgcgcta

SEQ ID NO: 129 - DNA, 16 bp, Synthetic construct
  source 1..16: mol_type = other DNA
gttccgtggccgtcgt

SEQ ID NO: 130 - DNA, 16 bp, Synthetic construct
  source 1..16: mol_type = other DNA
acagagcagaggaatc

SEQ ID NO: 131 - DNA, 16 bp, Synthetic construct
  source 1..16: mol_type = other DNA
cgaagtcgggttgccc

SEQ ID NO: 132 - DNA, 16 bp, Synthetic construct
  source 1..16: mol_type = other DNA
gggctcctgtccggcc

SEQ ID NO: 133 - DNA, 16 bp, Synthetic construct
  source 1..16: mol_type = other DNA
accgccccgagctccc

SEQ ID NO: 134 - DNA, 16 bp, Synthetic construct
  source 1..16: mol_type = other DNA
tacgggacagcccggg

SEQ ID NO: 135 - DNA, 16 bp, Synthetic construct
  source 1..16: mol_type = other DNA
aaagctgggagggaca

SEQ ID NO: 136 - DNA, 16 bp, Synthetic construct
  source 1..16: mol_type = other DNA
gggtagtcggcccccc

SEQ ID NO: 137 - DNA, 16 bp, Synthetic construct
  source 1..16: mol_type = other DNA
cgctctgtcgttccgc

SEQ ID NO: 138 - DNA, 16 bp, Synthetic construct
  source 1..16: mol_type = other DNA
atccgccttgcgggcc

SEQ ID NO: 139 - DNA, 16 bp, Synthetic construct
  source 1..16: mol_type = other DNA
aacgggacgagcacta

SEQ ID NO: 140 - DNA, 16 bp, Synthetic construct
  source 1..16: mol_type = other DNA
tgatgcaccagcgatc

SEQ ID NO: 141 - DNA, 16 bp, Synthetic construct
  source 1..16: mol_type = other DNA
ggccagctcaaggccc

SEQ ID NO: 142 - DNA, 16 bp, Synthetic construct
  source 1..16: mol_type = other DNA
agcaggcccaccaaca

SEQ ID NO: 143 - DNA, 16 bp, Synthetic construct
  source 1..16: mol_type = other DNA
acaggagggcggcgtt

SEQ ID NO: 144 - DNA, 16 bp, Synthetic construct
  source 1..16: mol_type = other DNA
tcattccacttcgtcc

SEQ ID NO: 145 - DNA, 16 bp, Synthetic construct
  source 1..16: mol_type = other DNA
acatggcgcacgtctc

SEQ ID NO: 146 - DNA, 16 bp, Synthetic construct
  source 1..16: mol_type = other DNA
acggctggcggagata

SEQ ID NO: 147 - DNA, 16 bp, Synthetic construct
  source 1..16: mol_type = other DNA
acccggtcacgcatgg

SEQ ID NO: 148 - DNA, 16 bp, Synthetic construct
  source 1..16: mol_type = other DNA
ggcccggtcgtacccg

SEQ ID NO: 149 - DNA, 16 bp, Synthetic construct
  source 1..16: mol_type = other DNA
ggtgtgagtccgttcc

SEQ ID NO: 150 - DNA, 16 bp, Synthetic construct
  source 1..16: mol_type = other DNA
cgtattgctcaccatg

SEQ ID NO: 151 - DNA, 16 bp, Synthetic construct
  source 1..16: mol_type = other DNA
aagctcccggtggata

SEQ ID NO: 152 - DNA, 16 bp, Synthetic construct
  source 1..16: mol_type = other DNA
ccaagggggccaaact

SEQ ID NO: 153 - DNA, 16 bp, Synthetic construct
  source 1..16: mol_type = other DNA
gactccccgggtcggc

SEQ ID NO: 154 - DNA, 16 bp, Synthetic construct
  source 1..16: mol_type = other DNA
tagtcccgagcgttcg

SEQ ID NO: 155 - DNA, 16 bp, Synthetic construct
  source 1..16: mol_type = other DNA
cttgctcgtgaagtag

SEQ ID NO: 156 - DNA, 16 bp, Synthetic construct
  source 1..16: mol_type = other DNA
agcggcgccggagatg

SEQ ID NO: 157 - DNA, 16 bp, Synthetic construct
  source 1..16: mol_type = other DNA
gtgccagatcgcgggc

SEQ ID NO: 158 - DNA, 16 bp, Synthetic construct
  source 1..16: mol_type = other DNA
ggagtctggaaggggc

SEQ ID NO: 159 - DNA, 16 bp, Synthetic construct
  source 1..16: mol_type = other DNA
gcgctgtcgctgccca

SEQ ID NO: 160 - DNA, 16 bp, Synthetic construct
  source 1..16: mol_type = other DNA
ggcgaccgccctctcg

SEQ ID NO: 161 - DNA, 16 bp, Synthetic construct
  source 1..16: mol_type = other DNA
tagggcggttcccctg

SEQ ID NO: 162 - DNA, 16 bp, Synthetic construct
  source 1..16: mol_type = other DNA
acgatctcgggggctg

SEQ ID NO: 163 - DNA, 16 bp, Synthetic construct
  source 1..16: mol_type = other DNA
ggaagcggggggagaa

SEQ ID NO: 164 - DNA, 16 bp, Synthetic construct
  source 1..16: mol_type = other DNA
cacctcgcccaggggt

SEQ ID NO: 165 - DNA, 16 bp, Synthetic construct
  source 1..16: mol_type = other DNA
cgggttgaaaggtatg

SEQ ID NO: 166 - DNA, 16 bp, Synthetic construct
  source 1..16: mol_type = other DNA
tctaagtggcatttga

SEQ ID NO: 167 - DNA, 16 bp, Synthetic construct
  source 1..16: mol_type = other DNA
cggacgagcaggacgt

SEQ ID NO: 168 - DNA, 16 bp, Synthetic construct
  source 1..16: mol_type = other DNA
gtctgcctgttttcta

SEQ ID NO: 169 - DNA, 16 bp, Synthetic construct
  source 1..16: mol_type = other DNA
catatgggcgtccggc

SEQ ID NO: 170 - DNA, 16 bp, Synthetic construct
  source 1..16: mol_type = other DNA
gccaacgagcgcctcc

SEQ ID NO: 171 - DNA, 16 bp, Synthetic construct
  source 1..16: mol_type = other DNA
cccagtcgcccagcgg

SEQ ID NO: 172 - DNA, 16 bp, Synthetic construct
  source 1..16: mol_type = other DNA
gcggtcgccagtcacg

SEQ ID NO: 173 - DNA, 16 bp, Synthetic construct
  source 1..16: mol_type = other DNA
cccccggggatacctg

SEQ ID NO: 174 - DNA, 16 bp, Synthetic construct
  source 1..16: mol_type = other DNA
gcagcgtggcgcgctc

SEQ ID NO: 175 - DNA, 16 bp, Synthetic construct
  source 1..16: mol_type = other DNA
tccctcgcgtccgcct

SEQ ID NO: 176 - DNA, 16 bp, Synthetic construct
  source 1..16: mol_type = other DNA
accccgtccctgcatc

SEQ ID NO: 177 - DNA, 16 bp, Synthetic construct
  source 1..16: mol_type = other DNA
ccgggtcaagctgacc

SEQ ID NO: 178 - DNA, 16 bp, Synthetic construct
  source 1..16: mol_type = other DNA
gacgagcctcccaggc

SEQ ID NO: 179 - DNA, 16 bp, Synthetic construct
  source 1..16: mol_type = other DNA
tagccgcgaggggccg

SEQ ID NO: 180 - DNA, 16 bp, Synthetic construct
  source 1..16: mol_type = other DNA
taccgctatcccagtc

SEQ ID NO: 181 - DNA, 16 bp, Synthetic construct
  source 1..16: mol_type = other DNA
actcctcccgctggta

SEQ ID NO: 182 - DNA, 16 bp, Synthetic construct
  source 1..16: mol_type = other DNA
gccggacgccggagag

SEQ ID NO: 183 - DNA, 16 bp, Synthetic construct
  source 1..16: mol_type = other DNA
cgcctttgcctgcagg

SEQ ID NO: 184 - DNA, 16 bp, Synthetic construct
  source 1..16: mol_type = other DNA
cagcgggaaacgctca

SEQ ID NO: 185 - DNA, 16 bp, Synthetic construct
  source 1..16: mol_type = other DNA
gccgctaccgcccgga

SEQ ID NO: 186 - DNA, 16 bp, Synthetic construct
  source 1..16: mol_type = other DNA
gtcggacatggcccca

SEQ ID NO: 187 - DNA, 16 bp, Synthetic construct
  source 1..16: mol_type = other DNA
gactacatcgcgcgcc

SEQ ID NO: 188 - DNA, 16 bp, Synthetic construct
  source 1..16: mol_type = other DNA
ccgtggaatgggcccc

SEQ ID NO: 189 - DNA, 16 bp, Synthetic construct
  source 1..16: mol_type = other DNA
gtagggggacgttgca

SEQ ID NO: 190 - DNA, 16 bp, Synthetic construct
  source 1..16: mol_type = other DNA
tgccgattgacctact

SEQ ID NO: 191 - DNA, 16 bp, Synthetic construct
  source 1..16: mol_type = other DNA
cgcgcgcgcctgtgcc

SEQ ID NO: 192 - DNA, 16 bp, Synthetic construct
  source 1..16: mol_type = other DNA
gaactagcgcggcggg

SEQ ID NO: 193 - DNA, 16 bp, Synthetic construct
  source 1..16: mol_type = other DNA
ctgtacgacggccacg

SEQ ID NO: 194 - DNA, 16 bp, Synthetic construct
  source 1..16: mol_type = other DNA
ttcggattcctctgct

SEQ ID NO: 195 - DNA, 16 bp, Synthetic construct
  source 1..16: mol_type = other DNA
gcccgggcaacccgac

SEQ ID NO: 196 - DNA, 16 bp, Synthetic construct
  source 1..16: mol_type = other DNA
cggtggccggacagga

SEQ ID NO: 197 - DNA, 16 bp, Synthetic construct
  source 1..16: mol_type = other DNA
cgtagggagctcgggg

SEQ ID NO: 198 - DNA, 16 bp, Synthetic construct
  source 1..16: mol_type = other DNA
ctttcccgggctgtcc

SEQ ID NO: 199 - DNA, 16 bp, Synthetic construct
  source 1..16: mol_type = other DNA
accctgtccctcccag

SEQ ID NO: 200 - DNA, 16 bp, Synthetic construct
  source 1..16: mol_type = other DNA
agcgggggggccgact

SEQ ID NO: 201 - DNA, 16 bp, Synthetic construct
  source 1..16: mol_type = other DNA
ggatgcggaacgacag

SEQ ID NO: 202 - DNA, 16 bp, Synthetic construct
  source 1..16: mol_type = other DNA
gcggggcccgcaaggc

SEQ ID NO: 203 - DNA, 15 bp, Synthetic construct
  source 1..15: mol_type = other DNA
cacttagtgctcgtc

SEQ ID NO: 204 - DNA, 15 bp, Synthetic construct
  source 1..15: mol_type = other DNA
ccgttgatcgctggt

SEQ ID NO: 205 - DNA, 17 bp, Synthetic construct
  source 1..17: mol_type = other DNA
gcatcagggccttgagc

SEQ ID NO: 206 - DNA, 16 bp, Synthetic construct
  source 1..16: mol_type = other DNA
tggcctgttggtgggc

SEQ ID NO: 207 - DNA, 16 bp, Synthetic construct
  source 1..16: mol_type = other DNA
ctgctaacgccgccct

SEQ ID NO: 208 - DNA, 17 bp, Synthetic construct
  source 1..17: mol_type = other DNA
cctgtggacgaagtgga

SEQ ID NO: 209 - DNA, 17 bp, Synthetic construct
  source 1..17: mol_type = other DNA
atgagagacgtgcgcca

SEQ ID NO: 210 - DNA, 17 bp, Synthetic construct
  source 1..17: mol_type = other DNA
tgttatctccgccagcc

SEQ ID NO: 211 - DNA, 17 bp, Synthetic construct
  source 1..17: mol_type = other DNA
gtccatgcgtgaccggg

SEQ ID NO: 212 - DNA, 17 bp, Synthetic construct
  source 1..17: mol_type = other DNA
tcgggtacgaccgggcc

SEQ ID NO: 213 - DNA, 15 bp, Synthetic construct
  source 1..15: mol_type = other DNA
ggaacggactcacac

SEQ ID NO: 214 - DNA, 17 bp, Synthetic construct
  source 1..17: mol_type = other DNA
ccatggtgagcaatacg

SEQ ID NO: 215 - DNA, 15 bp, Synthetic construct
  source 1..15: mol_type = other DNA
tatccaccgggagct

SEQ ID NO: 216 - DNA, 15 bp, Synthetic construct
  source 1..15: mol_type = other DNA
tagtttggccccctt

SEQ ID NO: 217 - DNA, 16 bp, Synthetic construct
  source 1..16: mol_type = other DNA
gggccgacccggggag

SEQ ID NO: 218 - DNA, 17 bp, Synthetic construct
  source 1..17: mol_type = other DNA
tccgaacgctcgggact

SEQ ID NO: 219 - DNA, 17 bp, Synthetic construct
  source 1..17: mol_type = other DNA
actacttcacgagcaag

SEQ ID NO: 220 - DNA, 17 bp, Synthetic construct
  source 1..17: mol_type = other DNA
catctccggcgccgctg

SEQ ID NO: 221 - DNA, 17 bp, Synthetic construct
  source 1..17: mol_type = other DNA
cccgcgatctggcacgc

SEQ ID NO: 222 - DNA, 15 bp, Synthetic construct
  source 1..15: mol_type = other DNA
cccttccagactcct

SEQ ID NO: 223 - DNA, 15 bp, Synthetic construct
  source 1..15: mol_type = other DNA
gggcagcgacagcgc

SEQ ID NO: 224 - DNA, 15 bp, Synthetic construct
  source 1..15: mol_type = other DNA
cgagagggcggtcgc

SEQ ID NO: 225 - DNA, 15 bp, Synthetic construct
  source 1..15: mol_type = other DNA
ccaggggaaccgccc

SEQ ID NO: 226 - DNA, 17 bp, Synthetic construct
  source 1..17: mol_type = other DNA
tacagcccccgagatcg

SEQ ID NO: 227 - DNA, 17 bp, Synthetic construct
  source 1..17: mol_type = other DNA
tttctccccccgcttcc

SEQ ID NO: 228 - DNA, 17 bp, Synthetic construct
  source 1..17: mol_type = other DNA
acccctgggcgaggtgc

SEQ ID NO: 229 - DNA, 17 bp, Synthetic construct
  source 1..17: mol_type = other DNA
atacctttcaacccgtc

SEQ ID NO: 230 - DNA, 17 bp, Synthetic construct
  source 1..17: mol_type = other DNA
aaatgccacttagaacg

SEQ ID NO: 231 - DNA, 17 bp, Synthetic construct
  source 1..17: mol_type = other DNA
tcctgctcgtccgtaga

SEQ ID NO: 232 - DNA, 17 bp, Synthetic construct
  source 1..17: mol_type = other DNA
aaacaggcagacgccgg

SEQ ID NO: 233 - DNA, 16 bp, Synthetic construct
  source 1..16: mol_type = other DNA
acgcccatatgggagg

SEQ ID NO: 234 - DNA, 15 bp, Synthetic construct
  source 1..15: mol_type = other DNA
cgctcgttggcccgc

SEQ ID NO: 235 - DNA, 17 bp, Synthetic construct
  source 1..17: mol_type = other DNA
tgggcgactgggcgtga

SEQ ID NO: 236 - DNA, 17 bp, Synthetic construct
  source 1..17: mol_type = other DNA
ctggcgaccgccaggta

SEQ ID NO: 237 - DNA, 17 bp, Synthetic construct
  source 1..17: mol_type = other DNA
tccccggggggagcgcg

SEQ ID NO: 238 - DNA, 17 bp, Synthetic construct
  source 1..17: mol_type = other DNA
ccacgctgcaggcggac

SEQ ID NO: 239 - DNA, 16 bp, Synthetic construct
  source 1..16: mol_type = other DNA
gcgagggagatgcagg

SEQ ID NO: 240 - DNA, 17 bp, Synthetic construct
  source 1..17: mol_type = other DNA
gacggggtggtcagctt

SEQ ID NO: 241 - DNA, 17 bp, Synthetic construct
  source 1..17: mol_type = other DNA
gacccgggcctgggagg

SEQ ID NO: 242 - DNA, 17 bp, Synthetic construct
  source 1..17: mol_type = other DNA
ctcgtccggcccctcgc

SEQ ID NO: 243 - DNA, 17 bp, Synthetic construct
  source 1..17: mol_type = other DNA
ggctagactgggatagc

SEQ ID NO: 244 - DNA, 17 bp, Synthetic construct
  source 1..17: mol_type = other DNA
ggtataccagcgggagg

SEQ ID NO: 245 - DNA, 17 bp, Synthetic construct
  source 1..17: mol_type = other DNA
agtctctccggcgtccg

SEQ ID NO: 246 - DNA, 16 bp, Synthetic construct
  source 1..16: mol_type = other DNA
gccctgcaggcaaagg

SEQ ID NO: 247 - DNA, 15 bp, Synthetic construct
  source 1..15: mol_type = other DNA
cgtgagcgtttcccg

SEQ ID NO: 248 - DNA, 17 bp, Synthetic construct
  source 1..17: mol_type = other DNA
ctgtccgggcggtagcg

SEQ ID NO: 249 - DNA, 17 bp, Synthetic construct
  source 1..17: mol_type = other DNA
gctggggccatgtccga

SEQ ID NO: 250 - DNA, 17 bp, Synthetic construct
  source 1..17: mol_type = other DNA
cggcgcgcgatgtagtc

SEQ ID NO: 251 - DNA, 17 bp, Synthetic construct
  source 1..17: mol_type = other DNA
ggggcccattccacggt

SEQ ID NO: 252 - DNA, 15 bp, Synthetic construct
  source 1..15: mol_type = other DNA
gcaacgtccccctac

SEQ ID NO: 253 - DNA, 17 bp, Synthetic construct
  source 1..17: mol_type = other DNA
agtaggtcaatcggcag

SEQ ID NO: 254 - DNA, 17 bp, Synthetic construct
  source 1..17: mol_type = other DNA
gcacaggcgcgcgcgcc

SEQ ID NO: 255 - DNA, 15 bp, Synthetic construct
  source 1..15: mol_type = other DNA
cgccgcgctagttcc

SEQ ID NO: 256 - DNA, 15 bp, Synthetic construct
  source 1..15: mol_type = other DNA
gtggccgtcgtacag

SEQ ID NO: 257 - DNA, 16 bp, Synthetic construct
  source 1..16: mol_type = other DNA
agcagaggaatccgaa

SEQ ID NO: 258 - DNA, 15 bp, Synthetic construct
  source 1..15: mol_type = other DNA
gtcgggttgcccggg

SEQ ID NO: 259 - DNA, 17 bp, Synthetic construct
  source 1..17: mol_type = other DNA
ctcctgtccggccaccg

SEQ ID NO: 260 - DNA, 15 bp, Synthetic construct
  source 1..15: mol_type = other DNA
ccccgagctccctac

SEQ ID NO: 261 - DNA, 16 bp, Synthetic construct
  source 1..16: mol_type = other DNA
gggacagcccgggaaa

SEQ ID NO: 262 - DNA, 16 bp, Synthetic construct
  source 1..16: mol_type = other DNA
gctgggagggacaggg

SEQ ID NO: 263 - DNA, 15 bp, Synthetic construct
  source 1..15: mol_type = other DNA
tagtcggcccccccg

SEQ ID NO: 264 - DNA, 15 bp, Synthetic construct
  source 1..15: mol_type = other DNA
ctctgtcgttccgca

SEQ ID NO: 265 - DNA, 15 bp, Synthetic construct
  source 1..15: mol_type = other DNA
tccgccttgcgggcc

SEQ ID NO: 266 - DNA, 15 bp, Synthetic construct
  source 1..15: mol_type = other DNA
acgggacgagcacta

SEQ ID NO: 267 - DNA, 15 bp, Synthetic construct
  source 1..15: mol_type = other DNA
atgcaccagcgatca

SEQ ID NO: 268 - DNA, 17 bp, Synthetic construct
  source 1..17: mol_type = other DNA
gccagctcaaggccctg

SEQ ID NO: 269 - DNA, 16 bp, Synthetic construct
  source 1..16: mol_type = other DNA
gcaggcccaccaacag

SEQ ID NO: 270 - DNA, 16 bp, Synthetic construct
  source 1..16: mol_type = other DNA
caggagggcggcgtta

SEQ ID NO: 271 - DNA, 17 bp, Synthetic construct
  source 1..17: mol_type = other DNA
tcattccacttcgtcca

SEQ ID NO: 272 - DNA, 17 bp, Synthetic construct
  source 1..17: mol_type = other DNA
aacatggcgcacgtctc

SEQ ID NO: 273 - DNA, 17 bp, Synthetic construct
  source 1..17: mol_type = other DNA
ggacggctggcggagat

SEQ ID NO: 274 - DNA, 17 bp, Synthetic construct
  source 1..17: mol_type = other DNA
ccgacccggtcacgcat

SEQ ID NO: 275 - DNA, 17 bp, Synthetic construct
  source 1..17: mol_type = other DNA
ttccggcccggtcgtac

SEQ ID NO: 276 - DNA, 15 bp, Synthetic construct
  source 1..15: mol_type = other DNA
atgggtgtgagtccg

SEQ ID NO: 277 - DNA, 17 bp, Synthetic construct
  source 1..17: mol_type = other DNA
gatacgtattgctcacc

SEQ ID NO: 278 - DNA, 15 bp, Synthetic construct
  source 1..15: mol_type = other DNA
actaagctcccggtg

SEQ ID NO: 279 - DNA, 15 bp, Synthetic construct
  source 1..15: mol_type = other DNA
gcccaagggggccaa

SEQ ID NO: 280 - DNA, 16 bp, Synthetic construct
  source 1..16: mol_type = other DNA
cggactccccgggtcg

SEQ ID NO: 281 - DNA, 17 bp, Synthetic construct
  source 1..17: mol_type = other DNA
tagtagtcccgagcgtt

SEQ ID NO: 282 - DNA, 17 bp, Synthetic construct
  source 1..17: mol_type = other DNA
gatgcttgctcgtgaag

SEQ ID NO: 283 - DNA, 17 bp, Synthetic construct
  source 1..17: mol_type = other DNA
cgggcagcggcgccgga

SEQ ID NO: 284 - DNA, 17 bp, Synthetic construct
  source 1..17: mol_type = other DNA
aggggcgtgccagatcg

SEQ ID NO: 285 - DNA, 15 bp, Synthetic construct
  source 1..15: mol_type = other DNA
gcccaggagtctgga

SEQ ID NO: 286 - DNA, 15 bp, Synthetic construct
  source 1..15: mol_type = other DNA
ctcggcgctgtcgct

SEQ ID NO: 287 - DNA, 15 bp, Synthetic construct
  source 1..15: mol_type = other DNA
ctgggcgaccgccct

SEQ ID NO: 288 - DNA, 15 bp, Synthetic construct
  source 1..15: mol_type = other DNA
tgtagggcggttccc

SEQ ID NO: 289 - DNA, 17 bp, Synthetic construct
  source 1..17: mol_type = other DNA
gaaacgatctcgggggc

SEQ ID NO: 290 - DNA, 17 bp, Synthetic construct
  source 1..17: mol_type = other DNA
gggtggaagcgggggga

SEQ ID NO: 291 - DNA, 17 bp, Synthetic construct
  source 1..17: mol_type = other DNA
gtatgcacctcgcccag

SEQ ID NO: 292 - DNA, 17 bp, Synthetic construct
  source 1..17: mol_type = other DNA
atttgacgggttgaaag

SEQ ID NO: 293 - DNA, 17 bp, Synthetic construct
  source 1..17: mol_type = other DNA
aggacgttctaagtggc

SEQ ID NO: 294 - DNA, 17 bp, Synthetic construct
  source 1..17: mol_type = other DNA
gttttctacggacgagc

SEQ ID NO: 295 - DNA, 17 bp, Synthetic construct
  source 1..17: mol_type = other DNA
gcgtccggcgtctgcct

SEQ ID NO: 296 - DNA, 16 bp, Synthetic construct
  source 1..16: mol_type = other DNA
agcgcctcccatatgg

SEQ ID NO: 297 - DNA, 15 bp, Synthetic construct
  source 1..15: mol_type = other DNA
cccagcgggccaacg

SEQ ID NO: 298 - DNA, 17 bp, Synthetic construct
  source 1..17: mol_type = other DNA
ccagtcacgcccagtcg

SEQ ID NO: 299 - DNA, 17 bp, Synthetic construct
  source 1..17: mol_type = other DNA
gggatacctggcggtcg

SEQ ID NO: 300 - DNA, 17 bp, Synthetic construct
  source 1..17: mol_type = other DNA
gtggcgcgctccccccg

SEQ ID NO: 301 - DNA, 17 bp, Synthetic construct
  source 1..17: mol_type = other DNA
tcgcgtccgcctgcagc

SEQ ID NO: 302 - DNA, 16 bp, Synthetic construct
  source 1..16: mol_type = other DNA
cgtccctgcatctccc

SEQ ID NO: 303 - DNA, 17 bp, Synthetic construct
  source 1..17: mol_type = other DNA
ggtcaagctgaccaccc

SEQ ID NO: 304 - DNA, 17 bp, Synthetic construct
  source 1..17: mol_type = other DNA
cgagcctcccaggcccg

SEQ ID NO: 305 - DNA, 17 bp, Synthetic construct
  source 1..17: mol_type = other DNA
agccgcgaggggccgga

SEQ ID NO: 306 - DNA, 17 bp, Synthetic construct
  source 1..17: mol_type = other DNA
taccgctatcccagtct

SEQ ID NO: 307 - DNA, 17 bp, Synthetic construct
  source 1..17: mol_type = other DNA
gactcctcccgctggta

SEQ ID NO: 308 - DNA, 17 bp, Synthetic construct
  source 1..17: mol_type = other DNA
gggccggacgccggaga

SEQ ID NO: 309 - DNA, 16 bp, Synthetic construct
  source 1..16: mol_type = other DNA
cacgcctttgcctgca

SEQ ID NO: 310 - DNA, 15 bp, Synthetic construct
  source 1..15: mol_type = other DNA
acagcgggaaacgct

SEQ ID NO: 311 - DNA, 17 bp, Synthetic construct
  source 1..17: mol_type = other DNA
cagccgctaccgcccgg

SEQ ID NO: 312 - DNA, 17 bp, Synthetic construct
  source 1..17: mol_type = other DNA
gccgtcggacatggccc

SEQ ID NO: 313 - DNA, 17 bp, Synthetic construct
  source 1..17: mol_type = other DNA
ccccgactacatcgcgc

SEQ ID NO: 314 - DNA, 17 bp, Synthetic construct
  source 1..17: mol_type = other DNA
ttgcaccgtggaatggg

SEQ ID NO: 315 - DNA, 15 bp, Synthetic construct
  source 1..15: mol_type = other DNA
tactgtagggggacg

SEQ ID NO: 316 - DNA, 17 bp, Synthetic construct
  source 1..17: mol_type = other DNA
gtgcctgccgattgacc

SEQ ID NO: 317 - DNA, 17 bp, Synthetic construct
  source 1..17: mol_type = other DNA
ggcgggcgcgcgcgcct

SEQ ID NO: 318 - DNA, 15 bp, Synthetic construct
  source 1..15: mol_type = other DNA
ccacggaactagcgc

SEQ ID NO: 319 - DNA, 15 bp, Synthetic construct
  source 1..15: mol_type = other DNA
tgctctgtacgacgg

SEQ ID NO: 320 - DNA, 16 bp, Synthetic construct
  source 1..16: mol_type = other DNA
cgacttcggattcctc

SEQ ID NO: 321 - DNA, 15 bp, Synthetic construct
  source 1..15: mol_type = other DNA
ggagcccgggcaacc

SEQ ID NO: 322 - DNA, 17 bp, Synthetic construct
  source 1..17: mol_type = other DNA
ggggcggtggccggaca

SEQ ID NO: 323 - DNA, 15 bp, Synthetic construct
  source 1..15: mol_type = other DNA
tcccgtagggagctc

SEQ ID NO: 324 - DNA, 16 bp, Synthetic construct
  source 1..16: mol_type = other DNA
cagctttcccgggctg

SEQ ID NO: 325 - DNA, 16 bp, Synthetic construct
  source 1..16: mol_type = other DNA
actaccctgtccctcc

SEQ ID NO: 326 - DNA, 15 bp, Synthetic construct
  source 1..15: mol_type = other DNA
agagcgggggggccg

SEQ ID NO: 327 - DNA, 15 bp, Synthetic construct
  source 1..15: mol_type = other DNA
cggatgcggaacgac

SEQ ID NO: 328 - DNA, 15 bp, Synthetic construct
  source 1..15: mol_type = other DNA
gcggggcccgcaagg

(19) United States
(12) Patent Application Publication
(10) Pub. No.: US 2023/0317208 A1
(43) Pub. Date: Oct. 5, 2023

(54) BIOPOLYMER SYNTHESIS

(71) Applicant: Ribbon Biolabs GmbH, Vienna (AT)

(72) Inventors: Harold P. Vladar, Vienna (AT); Luca Agozzino, Vienna (AT)

(21) Appl. No.: 18/193,934

(22) Filed: Mar. 31, 2023

Related U.S. Application Data
(60) Provisional application No. 63/325,685, filed on Mar. 31, 2022.

(51) Int. Cl. G16B 30/20; G16B 45/00

(52) U.S. Cl. CPC G16B 30/20; G16B 45/00

(57) ABSTRACT

The invention provides methods for synthesizing large biopolymers such as genome-scale polynucleotide molecules. Where genome-scale polynucleotides are to be made by attaching together a number of oligos, assembly of the desired polynucleotide from the oligos is represented as a tree, in which the branches represent nucleotide sequences and the nodes represent attachments between pairs of oligos. Numerous assembly trees are each generated and scored in silico according to how successful that tree will be in directing assembly of the desired polynucleotide, given biochemical properties of the oligos and proposed ligations. Systems and methods of the invention select a suitable tree and operate liquid handling systems to perform operations as shown by the selected tree to make the desired polynucleotide.

### SEQUENCE LISTING

A “Sequence Listing XML” is submitted herewith in XML file format and (i) the name of the file is RBIO-001-01WO.xml; (ii) the date of creation is Mar. 31, 2023; and (iii) the size of the file is 290,939 bytes and the material in the XML file is incorporated by reference.

### TECHNICAL FIELD

The invention relates to the synthesis of biopolymers.

### BACKGROUND

The Human Genome Project (HGP) was an international endeavor with the goal of determining the sequence of the human genome. Reading the sequence of the human genome relied on techniques such as gene mapping by restriction fragment-length polymorphism and DNA sequencing by di-deoxy chain terminator sequencing, often called Sanger sequencing. Using such techniques, a rough draft of the human genome was made in 2000 and a complete human genome was announced on Apr. 13, 2003.

In view of forward-looking demands in genomics that arise from the ability to read entire genomes, that project may be referred to as “HGP-read”. Now, “GP-write” has been formed—a new international consortium that seeks to use molecular synthesis, gene editing, and other technologies to engineer, make, and test living systems with the overarching goal of understanding the blueprint for life provided by HGP-read. One objective of GP-write is to accelerate synthesis techniques and reduce synthesis costs by 1000-fold within ten years. The GP-write project takes the position that to truly understand the genetic blueprint, it is necessary to “write” DNA and to build human and other genomes from scratch.

### SUMMARY

The invention provides methods for synthesizing large biopolymers, such as genome-scale polynucleotide molecules. In one aspect, the invention allows for the synthesis of genome-scale polynucleotides by using in silico graph structures to instruct the linear attachment of a large number of oligos. The invention provides methods to automatically parallelize the joining of smaller oligos into subparts for rapid and controlled ordering of the subparts, while avoiding unintended sequence structure. Methods are operable with oligos provided in containers such as multiwell plates and with a final desired polynucleotide sequence specified by, for example, a computer file.

The invention provides graph structures for directing the assembly of large polymers. While the invention will be exemplified below using a tree structure, it is intended that any suitable graph structure is useful for implementation of the methods described herein. In general, systems of the invention provide assembly of a desired polynucleotide sequence from the constituent oligos by assembling one or more trees, written as computer tree files, in which the branches and nodes represent combinations of liquid transfer steps, e.g., among wells of multi-well plates that are performed by a liquid handling system. Systems and methods of the invention automatically generate and score assembly trees according to how successfully the polynucleotide will be made when a given tree directs assembly by liquid handling systems. Systems and methods of the invention select a suitable tree and operate the liquid handling systems to make the desired polynucleotide.

Where the desired polynucleotide sequence is at least about 100 kb in length, and the oligos are short, e.g., less than about a few dozen bases in length, the number of potential trees showing steps for assembling the oligos into the polynucleotide is unfathomable to the human mind. Systems of the invention generate trees automatically and apply an algorithm to score the trees. A tree receives a score for the efficiency and fidelity with which the polynucleotide may be assembled. For instance, the score can account for how well sticky ends of oligos will faithfully anneal and ligate as intended, and also for how likely the sticky ends are to mis-ligate, and make unintended attachments. Systems of the invention can operate with very large trees in a recursive manner by making sub-trees and attaching those together. Systems of the invention may be tailored to instruments, consumables, and reagents used in liquid handling systems. For example, software modules that generate, score, and select trees can favor sub-trees that can be drawn wholly from one 96-well or 384-well plate to minimize plate changes during assembly. Similarly, the tree selection modules can favor wide, shallow trees (over deep and narrow ones), because shallow trees give the most opportunities for parallelism, allowing large numbers of segments of the polynucleotide to be synthesized in parallel. Results indicate that tree selection according to methods herein with resultant parallel synthesis can create in about five hours molecules that would take five days to synthesize with linear “part A then part B then part C” approaches.

An important insight of the disclosure is that tree selection for control of, for example, a liquid handling system can advance molecular synthesis beyond the concept of massive parallelism. As opposed to simple linear assembly, attaching oligos in order from the 5′ end to the 3′ end, the invention provides gains in efficiency by parallelizing assembly. For example, a desired sequence is split at the middle, each half is split at the middle, and so forth, and each resultant subpart is made in parallel and then those are attached together. The invention recognizes that a symmetrical assembly tree requires many ligation events that may not work well biochemically, which are disfavored, and also that reaction components may assemble in unwanted manners. Selecting a tree that avoids disfavored ligations may result in a highly asymmetric tree.

Avoiding disfavored ligations may also be addressed by an appropriate in silico partitioning of the desired sequence to identify constituent oligos and their sticky ends. Poorly-performing ligations may be avoided using methods of the invention to optimize the partitioning of desired sequence into constituted oligo sequences. Each partitioning will have its own set of oligos, sticky ends, and assembly trees. Methods of the invention may be used to evaluate a proposed partitioning by generating, scoring, and selecting a partitioning that gives oligos, sticky ends, and a tree that avoids pairs of oligos that will not ligate as intended. Separately, also, for a given partitioning and the associated set of oligos, methods may be used to select trees, including highly asymmetric trees, that by design avoid requiring reacting sets of oligos and sticky ends that will not perform as intended. To give one example, where segments A and B are intended to form AB (so that C can be added to form ABC), but are prone to form BA, the system selects an assembly tree that first forms BC and then mixes with A to form ABC. Such assembly trees are biased away from being symmetric, due to the computer system seeking out and writing trees that avoid disfavored ligations. In fact, asymmetry is an oversimplification. The selected tree for optimal assembly may have a topology that deviates from symmetry to such a degree that no mind could at once comprehend the tree geometry and no simple verbal description could be given to explain the tree geometry. The selected tree is designed with such specificity to the operations that are performed by the liquid handling system, that there is no single trait that can describe the tree. Selected trees will tend to be shallow and asymmetric, and they may tend to include sub-trees comprising, for example, 8 or 12 terminal taxa, but any given selected tree may have any topology.

As mentioned, disfavored ligations can be avoided by partitioning the desired polynucleotide sequence into constituent oligo sequences in a manner that optimizes the ends of the oligos according to the laboratory assembly/synthesis that will be performed. In a simplest case, the desired sequence is split in half repeatedly to identify constituent oligos (“partitioned”). Any given partitioning will give a set of oligo sequences with specified ends. Those ends (sticky or blunt) may not work well when the oligos are joined to make the molecule. For example, sticky-ended oligos may have sticky ends that form GC rich hairpins, and do poorly at ligating together. Or an oligo may have two complementary sticky ends meaning that the oligo is prone to ligating to copies of itself.

The invention includes methods for partitioning in silico the desired polynucleotide sequence into constituent oligo sequences, creating assembly trees for those sequences, and scoring the trees with score sets that predict how the ends of those oligos will perform. In such embodiments, each partitioning of a desired polynucleotide sequence into constituent oligos will have its own set of assembly trees. Scoring and selecting trees automates the process for choosing a partitioning, which identifies the oligos that will be used in making the polynucleotide. By doing so, the liquid handling system can be provided with oligos that will perform as intended when making the polynucleotide. Due to the in silico partitioning, the laboratory systems will reliably make the intended product without making unintended product.

By using the in silico methods and system described herein to make polynucleotides, those molecules can be made rapidly, with a high degree of parallelization in liquid handling systems. Poorly-performing constructs are avoided, so those molecules are made reliably. Methods and systems of the invention are being used for making polynucleotides greater than 100,000 base pairs in length. Typical constituent oligos comprise a pair of single DNA strands of length up to, e.g., 50-100 bases that overlap by a number of bases, leaving sticky ends at each end. Such oligos each contribute about 8 bases to the final molecule. When those are assembled together, there are multiple trillions of potential “trees” describing the order and parallelization of steps to make one molecule from one set of oligos. Systems and methods of the invention can rapidly create many such trees, score the trees, select one tree, and then operate the liquid handling systems according to the selected tree to create a genome-scale molecule. Thus the invention provides for the rapid and reliable writing of genomic-scale DNA.

In certain aspects, the invention provides a method of polymer synthesis. The method includes inputting a desired polynucleotide sequence into a computer system, e.g., as an in silico graph structure comprising a plurality of branches, each of which specifies a linear order of nucleic acids. The in silico graph structure is programmed for selecting an optimal combination of branches that specify the desired sequence and for directing the assembly of the polynucleotide molecule with the desired sequence. Preferably the branches correspond to subsets of the polynucleotide or oligos provided in separate compartments, such as wells of multi-well plates. The in silico graph structure may be resident in a computer system comprising program instructions executable to cause the system to: generate a plurality of trees, each tree representing an ordered combination of branches, resulting in the desired polynucleotide sequence; select the tree that provides the optimal combination of branches; and direct the assembly of the desired polynucleotide sequence using the selected tree structure. Generally, the trees will include branches connected by nodes that represent attachments between the oligos or sub-polynucleotides. The selecting step may include calculating a score for each tree, the score representing a measure of success that the tree structure will result in assembly of the molecule with the desired polynucleotide sequence. The measure of success may be based on stored scores or probabilities of success in assembling partial constructs and/or stored probabilities of mis-assemblies.

Certain embodiments use recursive assembly, in which the selecting step includes identifying subgroups (of, e.g., fewer than about twenty to one hundred oligos); generating, scoring, and selecting an optimal subtree for each subgroup; and combining optimal subtrees from the subgroups to form an assembly tree having an optimal production score.

Preferred methods may include directing fluidic handling system to make the desired polynucleotide. The fluidic handling system may be a multi-channel robotic system, an acoustic liquid handling system, or a microfluidic system.

Embodiments of the computer system may use a software package to search tree space and identify local maxima therein, thereby to select one or more trees from the local maxima. Methods may include joining subgroups of branches together to form a branch path comprising a desired polynucleotide sequence. Preferably the selecting algorithm selects for a nested order of combinations among oligos that successfully produces the desired polynucleotide. The computer system may execute a machine learning algorithm to select said optimal combination of branches. The machine learning algorithm may use one or a combination of: a decision tree algorithm, a random forest algorithm, an extreme gradient boosting algorithm, an adaptive boosting algorithm, a deep learning algorithm. The computer system may perform search and optimization using Bayesian algorithms, genetic algorithms, and Monte Carlo analyses, for example.

In related aspects, the invention provides a system for polymer synthesis. The system comprises a computer system operable to receive a desired polynucleotide sequence e.g., into an in silico graph structure comprising a plurality of branches, wherein each branch specifies a linear order of nucleic acids. The computer system is programmed for selecting an optimal combination of branches that specify the desired sequence. The system includes a liquid handling system and the computer system is programmed for directing the liquid handling system to assemble the polynucleotide molecule with the desired sequence.

In other aspects, the invention provides a method of synthesizing a polymer. The method comprising: receiving information describing a desired polynucleotide; generating a plurality of trees, each tree giving an order of attachments among oligos to form the polynucleotide; selecting one of the trees having an optimal production score; and making the polynucleotide by joining the oligos in as given by the selected tree. Optionally, leaves of the trees represent the oligos or overhangs thereof and nodes of the trees represent attachments between oligos. The selecting step may include calculating a score for each tree, the score representing a probability that the polynucleotide will be successfully made using that tree. The method may include scoring the tree using (i) stored probabilities of success in ligating overhangs of the oligos; (ii) stored estimates of risk of mis-ligations between unintended pairs of the oligos; or (iii) both.

There may be greater than about 10\hat{ }30 trees that give an order of attachments among the oligos to form the polynucleotide, and, in certain recursive assembly embodiments, the method may include performing the generating and selecting steps for a first subset of the oligos to form an intermediate tree; and storing the intermediate tree for use as a leaf in a final tree having the optimal production score (i.e., not scoring all 10\hat{ }30 trees, by performing the steps on nest sub-parts of the final tree). For example, the method may include identifying subgroups of the oligos (e.g., each comprising fewer than about twenty to 100 of the oligos); generating, scoring, and selecting sub trees for each subgroup; and joining together one optimal scoring tree from each subgroup to yield the selected tree having the optimal production score. For each subgroup, a software package may generate all possible trees; store some or all of the trees in memory; apply a scoring matrix to each of the trees in memory to score all of the trees of that subgroup; and select a best-scoring tree for the sub-group.

Some methods include the step of making the molecule, i.e., the polynucleotide. Making the polynucleotide may include making one substantially entire expression vector or organismal genome, e.g., of at least about 100 kb. In some embodiments, the method includes performing the steps to produce a library of variants.

In certain embodiments, making the polynucleotide includes executing a software package to transform the selected tree into instructions executed by a liquid handling system to transfer the oligos among storage vessels (e.g., tubes or wells of multi-well plates), in the order given by the selected tree to make the polynucleotide. Optionally, a mapping software package creates a map of the selected tree onto hardware features of the liquid handling system, e.g., creates a description of an arrangement of the oligos in a multi-well plate wherein the described arrangement minimizes steps or operations of the liquid handling system making the polynucleotide when joining the oligos in the order given by the selected tree.

The selected tree may be asymmetric (a first leaf is connected to a root of the selected tree by a first number of nodes and edges and a second leaf is connected to the root of the selected tree by a second number not equal to the first). Selecting the tree may operate by searching tree space, wherein for at least one subgroup of the oligos, a tree search software packages searches tree space to identify a local maxima in tree space and select the best scoring tree. The tree search software packages may use an exhaustive search strategy, heuristic searches, genetic algorithms, a Monte Carlo search strategy, a Bayesian search strategy, maximum likelihood calculations, or a machine learning algorithm. The steps may be performed to create assembly trees for multiple subgroups of the oligos. Selecting the tree with the optimal production score may include calculating probabilities of successful ligation and also promoting trees that promote parallelization on liquid handling systems (i.e., favoring wide, shallow trees).

Other aspects provide a system for synthesizing a polymer. The system includes a computer system operable to receive information describing a desired polynucleotide and generate a plurality of trees, each tree giving an order of attachments among oligos (preferably that are provided in vessels such as wells) to form the polynucleotide. The computer selects one of the trees having an optimal production score and directs a liquid handling system of the system to make the polynucleotide by joining the oligos according to the structure of the selected tree.

### BRIEF DESCRIPTION OF THE DRAWINGS

FIG. 1 shows a hypothetical simple assembly tree.

FIG. 2 diagrams a method of polymer synthesis.

FIG. 3 is a graphical depiction of a symmetrical assembly tree for a short sequence.

FIG. 4 is a graphical depiction of an asymmetrical tree for the short sequence.

FIG. 5 shows a matrix of scores.

FIG. 6 shows a plurality of oligos that may be used.

FIG. 7 shows a ligation matrix.

FIG. 8 shows a mapping matrix.

FIG. 9 shows a scoring matrix that combines the ligation matrix and the mapping matrix.

FIG. 10 shows symmetrical and asymmetrical assembly trees for a sequence.

FIG. 11 shows different assembly trees and their corresponding matrices.

FIG. 12 illustrates recursive optimization.

FIG. 13 shows a fully optimized tree that results from recursive assembly.

FIG. 14 illustrates components that may be included in a system of the invention.

FIG. 15 shows an adjacency matrix for a specific example.

FIG. 16 shows a graphical representation of a scoring.

FIG. 17 shows that each tree gives a corresponding score.

FIG. 18 gives the optimal assembly tree for one example.

FIG. 19 shows the overhang adjacency matrix for the tree of FIG. 18.

FIG. 20 shows an alternative assembly tree

FIG. 21 gives a corresponding adjacency matrix.

FIG. 22 gives an adjacency matrix with scoring S1.

FIG. 23 shows the assembly tree with the S1 scoring.

FIG. 24 shows an adjacency matrix with scoring S2.

FIG. 25 shows the assembly tree with S2 scoring.

FIG. 26 shows an adjacency matrix with scoring S3.

FIG. 27 gives the assembly tree for the S3 scoring.

FIG. 28 shows a symmetric assembly.

FIG. 29 show an asymmetric assembly.

FIG. 30 gives electropherograms for two assemblies.

FIG. 31 shows steps of a method.

FIG. 32 shows a scored assembly tree for a naïve partition with a symmetric tree,

FIG. 33 shows a scored assembly tree for a naïve partition with the asymmetric tree and

FIG. 34 shows a scored assembly tree for an adaptive partition with a symmetric tree.

FIG. 35 shows products of ligation and misligation matrices.

FIG. 36 shows a minimal example of likelihood components in an assembly tree.

FIG. 37 shows three alternative examples of priors following a Beta distributions.

### DETAILED DESCRIPTION

The invention provides methods of making biopolymers having a desired sequence of interest. For example, it may be desired to make one contiguous DNA molecule of a predetermined sequence in which the molecule is of some arbitrarily long length, such as greater than 100,000 base pairs. In other examples, it may be desired to make one or a family of variants sharing certain defined sequence characteristics, and thus sharing certain sequence similarities, but in which the variants do not match one another exactly. For example, it may be desired to make a plurality of variants of a vector for delivery of a gene or operon in which the variants have different regulatory elements, or different locations or orders of regulatory elements, so that the variants can be tested and evaluated for delivery and expression of the gene(s). In that sense, the invention provides methods for making one or any number of desired polynucleotide sequences, any or all of which may have a sequence that is completely defined, or as variants that embody certain defined parameters.

An issue that arises in the making of arbitrarily long biopolymers involves the methods by which to actually make the molecules. A common theme among methods of making the molecules is to obtain a plurality of subunits of the molecules and then attach those subunits to one another piecewise until the long molecule is made. When the subunits are much shorter than the desired biopolymer, e.g., when making a long (>tens of thousands of bases in length) nucleic acid from oligonucleotides, or “oligos”, which are between a few and few tens of bases in length, there may be different approaches to attaching the subunits together.

For example, in what may be dubbed a linear approach, the constituent oligos may be identified in the 5′ to 3′ direction and, at step 1, the second oligo is introduced, and ligated, to the 5′-most oligo (which may have its 5′ end blocked, e.g., by chemical capping or solid phase attachment). At step 2, the third oligo is introduced to, and ligated to, the ligation product of step 1. At each subsequent step, the next oligo is ligated to the emerging polynucleotide. This linear approach is appealing in its simplicity and the variables that must be controlled to avoid mis-assembly appear to be few. However, the linear approach is time-consuming. If one seeks to make a 100,000 base polynucleotide for 8-mer oligos, there will be 12,500 steps, where each step requires pipetting from one container of oligos into another, introducing ligase, factors, incubating, washing away excess reagents, re-suspending in saline or buffer, and then moving on to the next step. The 100 k polynucleotide appears to be quite time-consuming to make.

Another approach, dubbed hierarchical assembly, could involving attaching different adjacent pairs of oligos from distal locations along the final desired polynucleotide sequence to make an intermediate tiers of two-part oligos (e.g., joining 8-mers to form 16-mers), or sub-polynucleotides. Then, the two-part oligos could be joined pair-wise (e.g., to form 32-mers). Those joining steps may proceed in a pair-wise fashion to make larger sub-parts of the desired polynucleotide sequence at each tier of assembly. Hierarchical assembly is appealing because it allows many of the steps to be parallelized. To illustrate, when the starting oligos are 8-mers, step 1 produces 16-mers, step 2 produces 32-mers, and step 3 produces 64-mers. The length produced by each step is potentially double the length produced by the prior step and, in theory, the fifteenth step produces a polynucleotide with a length of 131,072 bases. Compared to the 12,500 sequential steps required in the linear approach, a 100,000 base polynucleotide could be assembled with 15 sequential steps by the hierarchical approach. However, successful assembly by the hierarchal approach raises potential issues. For example, if a pair of 8-mer oligos are to be pipetted together from two reaction containers, to correctly form a desired polynucleotide sub-sequence, that pair of oligos must join in the correct order and not to themselves. Thus the possible combinations of steps available under hierarchical assembly are limited to pairwise combinations in which the free ends of a pair of oligos intended to be joined are biochemically conducive to being joined, and the unintended ends are not biochemically susceptible to being joined.

Hierarchical assembly may be accomplished using oligos that are biochemically amenable to being joined at their intended ends in a desired order. For example, if first and second 8-mers A and B are intended to form the 16-mer AB, then it is preferable to use oligos that do not react biochemically to form any unintended product such as BA, AAAAA, ABBB, ABAB, etc. This may be accomplished by designing and providing oligos with appropriate sticky ends. For example, if A, B, C, D, E, and F are to be joined to form ADEBCDEF (these are not IUPAC nucleotide codes or amino acid codes; these are just letters representing hypothetical oligos), then A must have a sticky end that anneals to a sticky end of D. Similarly, D must have a sticky end that anneals to a sticky end of A and a sticky of C in the intended order.

For a given desired polynucleotide sequence, it is possible to bioinformatically identify constituent oligos with sticky ends that will anneal. Making one complete division of the desired polynucleotide sequence into its constituent oligos with sticky ends identified may be called partitioning the desired sequence. If the constituent oligos are to be 16-mers, while it would appear that there are greater than 4 billion possible 16-mers, there are only about 6,250 unique 16-mers in a polynucleotide of 100,000 bases in length. That number of oligos can be created and stored in, and then dispensed from, about 66 96-well plates, which is a tractable number. Similarly, that number of oligos may be provided in, and dispensed from, about 17 384-well plates. Thus it may be that one may use a computer system to partition the sequence from a desired polynucleotide sequence about 100,000 bases in length to identify about 6,000 unique oligos with sticky ends that will only anneal uniquely to intended sticky ends among the oligos. Those oligos may be synthesized or ordered and provided in reaction containers such as within the wells of about 17 different 384-well plates. With the constituent oligos thus provided, it would appear that assembling the desired polynucleotide sequence may proceed in a straightforward manner. In a first step, (identifying the oligos by their positions along the desired polynucleotide sequence) the first oligo is pipetted into the second, while the third is pipetted into the fourth, and the fifth is pipetted into the sixth and so on. The pairs are attached (e.g., ligated) to form sub-polynucleotides. At the completion of the first step, there are sub-polynucleotides double the length of the oligos, and the process is repeated iteratively, treating the sub-polynucleotides in the same manner as the oligos. In fact, if the process proceeds to completion in a perfectly iterative fashion, a map or graph of all of the assembly steps is symmetrical.

The pairwise assembly of oligos into a desired polynucleotide sequence may be described using a map or graph, which has the structure of a tree. A tree is a structure made up of branches and nodes. A tree may be used to describe assembly of a desired polynucleotide sequence from oligos by using branches of the tree to represent nucleotide sequences and nodes to represent attachments between pairs of the nucleotide sequences. In that representation, the root of the tree is a branch representing the desired sequence connecting at a root node that represents the final attachment operation (e.g., ligation) that actually forms the desired polynucleotide sequence. The terminal branches (or “terminal taxa”) represent the starting constituent oligonucleotides from which the desired polynucleotide sequence will ultimately be made. Internal branches represent sub-polynucleotides, shorter than the desired polynucleotide sequence, but having been made by joining the starting oligonucleotides.

A benefit of trees in the context of making a desired polynucleotide sequence from oligos, is that a tree can be represented in a digital file. The ability to represent trees in a digital file allows a number of things to happen. First, software systems can generate trees automatically using logical instructions embodied in software program instructions. Secondly, software systems can automatically evaluate, compare, or score trees. Finally, software systems can read a tree and execute programed instructions to direct the operation of automated hardware, such as a liquid handling system in a laboratory.

Any suitable format may be used to store and represent trees such as, for example, a graph database. Graph databases are examples of software systems that represent nodes and edges using node and edge objects and either index-free adjacency or adjacency lists to store and represent relationships among nodes and edges. In another example, trees could be stored and represented using a directory structure of a filesystem. For example, for 4 8-mer oligos (e.g., A, B, C, and D to become ABCD) that will be assembled to a 32-mer desired polynucleotide sequence, each oligo could be a FASTA file in a Linux-type file system with nested directories representing tree structure. The paths could be:
/output
/output/subpart2
/output/subpart1
/output/subpart2/D.fa
/output/subpart2/C.fa
. . . etc.

A tree can also be represented graphically, in a figure or drawing.

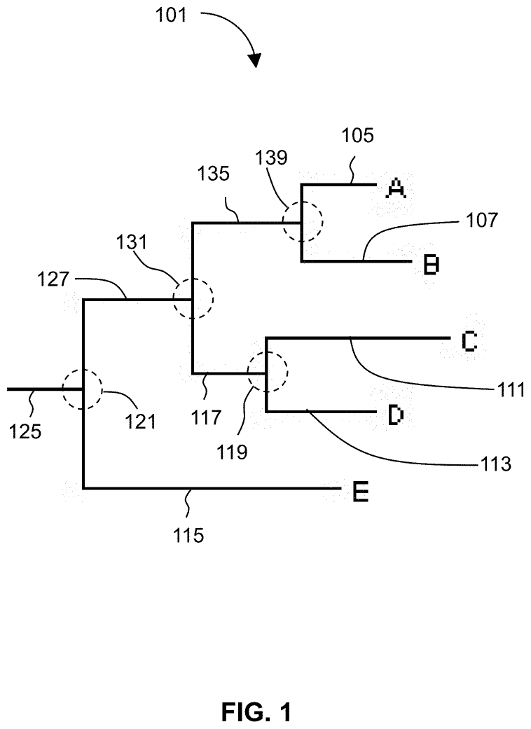

FIG. 1 is a drawing of a tree 101 for joining oligos named A, B, C, D, and E. In tree 101, terminal branch 105 represents oligo A, terminal branch 107 represents oligo B, terminal branch 111 represents oligo C, terminal branch 113 represents oligo D, and terminal branch 115 represents oligo E. Node 119 represents a step, or action, in which a 3′ end of oligo D will be attached, or joined, to a 5′ end of oligo C to form sub-polynucleotide DC. Branch 117 represents sub-polynucleotide DC. Node 139 represents a step at which a 3′ end of oligo B will be joined to a 5′ end of oligo A to form sub-polynucleotide AB. Branch 135 represents sub-polynucleotide AB. Node 131 represents the attachment of sub-polynucleotide DC to AB, to form sub-polynucleotide DCBA, which is represented by branch 127. Branch 115 represents oligo E. Node 121 represents the attachment of oligo E to sub-polynucleotide DCBA. Thus, root broach 125 represents the product of the attachment of E to DCBA, which is desired polynucleotide sequence EDCBA.

There are a few important features of trees such as tree 101. First, they represent an order or combination of operations for forming a described polynucleotide sequence from constituent oligos. Referencing tree 101, each of oligos A, B, C, etc., may be provided within vessels such as a well of a multiple well plate. Each well may contain an arbitrarily high number (e.g., millions) of clonal copies of the one oligo. Tree 101 as drawn represents that D is joined to C and B is joined to A in parallel, before DC is joined to BA. Thus, in particular, tree 101 informs a method of polymer synthesis by showing that D will be joined to C while B is joined to A, only after which DC is joined to BA. Given a desired polynucleotide sequence EDCBA, the tree 101 informs that the method should not be performed in a naïve order of joining D to E, then joining C to ED, then joining B to EDC, and final joining A to EDCB. A second important feature of trees is that they can be evaluated or compared to one another, which is discussed further below. Finally, another important feature of tree is that they can be representing in a computer file, such as a plain text file, representing the tree as a text string, using only ASCI characters, allowing trees to be saved in a compact format and readily retrieved, manipulated, or evaluated.

For example, in some embodiments, a tree is stored in a text-formatted file such by using an optionally-modified implementation of the New Hampshire/Newick format. The Newick file format, also referred to as the New Hampshire format, relies on strings of text in order to encode tree representations. The subparts of a tree are represented by codes or text strings, and a set of nested brackets denotes the tree structure. The Newick format has additional features, such as the encoding of branch lengths, however, those features are not necessary at this point of discussion. For a discussion of the Newick format, see Pavlopoulos, 2010, A reference guide for tree analysis and visualization, Bio Data Min 3:1, incorporated by reference. The Newick Standard for representing trees in computer-readable form makes use of the correspondence between trees and nested parentheses. Given the tree 101 shown graphically here, the Newick standard presents the tree using the following sequence of printable characters: (E, ((D, C),(B,A))));

The Newick tree ends with semicolon. Because the desired polynucleotide sequence is pre-determined to be EDCBA, the edge 125 is root not a tip, and node 121 is an internal node. Tip and terminal edge, or terminal taxa, are synonymous. Interior nodes are represented by a pair of matched parentheses. Between them are representations of the nodes that are immediately descended from that node, separated by commas. As used herein, trees may be written in an ordered tree file, which is a modified version of the Newick Standard. The Newick Standard does not require that the left-right order of descendants of nodes (e.g., including the terminal tips) have any meaning. In a true Newick Standards, (A,(B,C),D); is the same tree as (A,(C,B),D). Also, the Newick Standard does not, strictly speaking, require a root. Here, the disclosure may use a modified tree file in which the left-right order of nodes has meaning and the tree is, of necessity, rooted (where the root maybe the left-to-right reading of terminal taxa). The root of the tree is the desired polynucleotide sequence. Accordingly, (E, ((D, C),(B,A)))); is a correct modified Newick tree file for the tree 101 and the desired polynucleotide sequence, EDCBA, is the root and can be read from the modified tree file (“modified” in that the tree file is a specific implementation of the Newick format).

A benefit of storing trees in such a text format is that the tree file may be used to control operation of a liquid handling system, sometimes referred to as or including a liquid handling robot or robotic liquid handler. Any suitable type of liquid handling system may be used. Some versions of liquid handling robots dispense an allotted volume of liquid from a motorized pipette or syringe, while some such systems manipulate the position of the dispensers and containers (often a Cartesian coordinate robot, such as the XYZ Triton Robot from TriContinent Scientific) and/or integrate additional laboratory devices, such as centrifuges, microplate readers, heat sealers, heater/shakers, bar code readers, spectrophotometric devices, storage devices and incubators. Exemplary liquid handling systems include bench-top 8-channel DNA processing robots and customized-for-process automated liquid handling systems, such as the TECAN Freedom EVO, the automated liquid handler sold under the trademark PRIME by HighRes Biosolution or the automated liquid handler sold under the trademark JANUS by PerkinElmer.

Other versions of liquid handling systems may be used including “pickers” or “cherry pickers, which include liquid handlers that mimic the operations of humans, by performing liquid transfers using cartesian, 3-axis movements implemented in larger workstations, e.g., by means of an arm. Such cherry pickers include the pipetting robot sold under the trademark ANDREW+ by Waters Corporation (Milford, MA) or the TOMTEC QUADRA 3 cherry picker automated liquid handling system from Tomtec, Inc. (Hamden, CT).

Certain embodiments make use of an acoustic system such as the acoustic liquid handler sold under the trademark ECHO 650 by Beckman Coulter Life Sciences (Indianapolis, IN). In an acoustic liquid handler, a multiwell source plate containing reagents is placed on a source plate gripper. A destination plate is placed on a destination plate gripper, that will invert the destination plate and position it above the source plate. Mechanical motors within the handler will position a source well beneath a destination well. A transducer is placed beneath the source well, typically aqueously coupled to the bottom of the source well in water. The liquid handler can use the transducer to measure a distance to a well bottom and meniscus in the source plate, and then deliver a burst of ultrasonic energy calculated by the handler to eject a droplet of liquid from the source well on a trajectory to impact a bottom surface of the inverted destination well above. Liquid handlers such as the ECHO 650 reliably operate to transfer liquid volumes on the order of 2.5 nL. Surface tension retains the transferred liquid on the bottom of the well of the inverted destination plate. Larger volumes may be transferred by a series of acoustic bursts.

Whether the liquid handling system uses a multi-channel processing robot, a cherry picker, or an acoustic liquid handler, what such liquid handling systems have in common is that they can typically be operated under computer control. A computer control system may store information about reagents and products and instructions for operating the liquid handling system. The computer control system may include one or any combination of: a computer built into the liquid handling system; a computer workstation operatively connected to the liquid handling system; a server or cloud computing resource; and a connected network computer, e.g., on a LAN or over WiFi or the Internet. The computer control system can read from a tree file and direct operation of the liquid handling system to synthesize a part of, or all of, a desired polynucleotide sequence.

The computer control system accomplishes synthesis of (at least a part of) the desired polynucleotide sequence by instructing the liquid handing system to operate to perform the assembly steps represented by the assembly tree.

If the desired polynucleotide sequence is made by hierarchal assembly, then perhaps the simplest example of a perfectly iterative assembly is that described by a symmetrical tree. An important insight of the invention is that the symmetrical tree may not, and likely usually does not, describe the best way to create the desired polynucleotide in the laboratory. One issue is that the symmetrical tree is naïve of numerous factors such as reagent biochemistry, opportunities to exploit sequence content of the desired polynucleotide sequence, or practical considerations of real-world laboratory hardware. For example, multiwell plates often have reagents in rows or columns that include wells in a multiple of 8 or 12, and various laboratory instruments are designed to pipette from or to 8, 12, 16, 24, etc., wells in one action. Depending on laboratory equipment used, a symmetrical assembly tree may need to be broken out into parts congruent with the rows or columns of those plates and may also require leaving wells empty.

Perhaps more significantly, even uniquely-matched sticky ends may not always anneal with perfect fidelity and non-matched sticky ends may sometimes anneal to one another. Also, importantly, certain sticky ends—even if they make for a unique match—have properties that interfere with successfully synthesizing a desired polynucleotide sequence. For example, if a sticky end has internal regions that are self-complementary, that region may form hairpins. The 5′ GC of a sticky end that includes GCAAAACG may anneal to the 3′ CG, rendering the end unavailable to another oligo. Or, if a sticky end is extremely AT- or GC-rich, it may anneal or not under conditions in which other paired oligos in the multiwell plate are intended to be doing the opposite.

An insight of the disclosure is that one assembly tree represents one set of an ordered combination of steps by which a desired polynucleotide sequence can be made via the pairwise attachment of constituent oligos. Also, there are a large number of sets of ordered combinations of steps by which a desired polynucleotide sequence can be made via the pairwise attachment of constituent oligos. This means that there are a large number of assembly trees for a given desired polynucleotide sequence.

Given that one object of the disclosure is to provide methods by which to successfully make a desired polynucleotide sequence, the disclosure provides methods by which to select a suitable assembly tree. Stated briefly, those methods involve generating a large number of trees that represent sequence partitioning into oligos and represent making the desired polynucleotide from sets of the oligos, scoring those trees based on practical and biochemical criteria that are applied to features of the trees, selecting a tree that obtains a score indicating the polynucleotide will be successfully made to specifications (i.e., selecting a tree that includes an optimal partitioning and an optimal combination of branches that specify the desired sequence), providing a set of oligos indicated by the partitioning, and executing program instructions that direct a liquid handling system to assemble the polynucleotide molecule with the desired sequence.

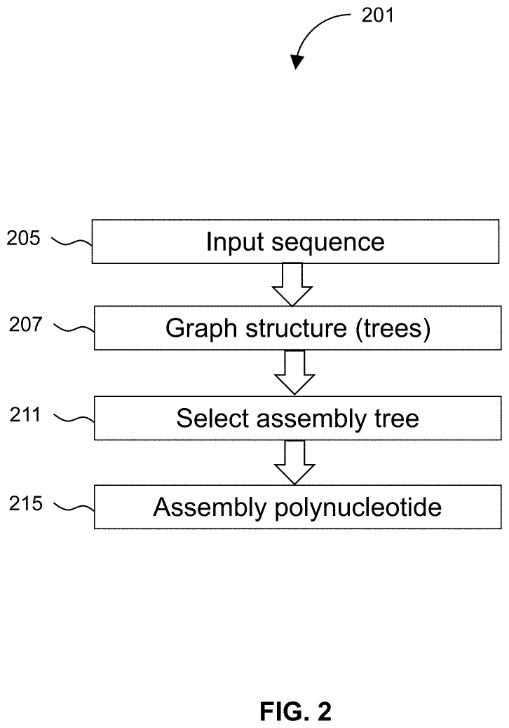

FIG. 2 diagrams a method 201 of polymer synthesis. The method 201 includes inputting 205 a desired polynucleotide sequence in an in silico computer system. The desired sequence maybe input in any suitable format, such as a simple text string, or as a FASTA or FASTQ file. The sequence may be input 205 into a computer system by receiving the sequence, e.g., over the Internet. That is, the polynucleotide may be synthesized, or made, at a facility or lab, that receives the desired polynucleotide sequence as an order, e.g., via a web form or email. A next step is to populate or instantiate 207 in silico graph structures. There are a variety of steps or approaches that may be used. For example, nucleic acid constituents of the desired polynucleotide sequence may be represented using a graph database, such as by Neo4J. In certain embodiments, graph structures are generated that are made up of branches, such that each graph structure specifies a linear order of nucleic acids. In a preferred embodiment, the computer system creates or has stored therein a sequence of each of a plurality of constituent oligos that could be used to make up the desired polynucleotide sequence. Programming logic operates to select an optimal combination of branches that specify the desired sequence (which will be used for directing the assembly of the polynucleotide molecule with the desired sequence).

In the preferred embodiment, instantiating 207 the graph structures may include generating a plurality of assembly trees (such as, e.g., tree 101). Each tree represents one set of an ordered combination of steps by which a desired polynucleotide sequence can be made via the pairwise attachment of constituent oligos and the sub-polynucleotides that result from first rounds of attachment. The trees can optionally be generated indiscriminately with respect to the assembly product they represent, because trees that represent an assembly product inconsistent with the desired polynucleotide sequence can be deleted at the software level. What is important is that, of all the assembly trees that are generated, a number of them would, if executed, result in the desired polynucleotide sequence.

The method 201 include selecting 211 a tree structure with an optimal combination of branches. Selecting 211 a tree by methods that are conducive to automation are discussed below. Once the tree is selected 211, the method 201 includes assembling 215 the polynucleotide, typically be executing computer program instructions that read the selected tree and direct laboratory instruments to manipulate reagents to form the desired polynucleotide sequence.

Selecting 211 a tree involves choosing an assembly tree (from among numerous such trees) that will give the laboratory instruments a high probability to success in forming the desired polynucleotide sequence. Some trees may not be associated with a high probability of success because specific attachment reactions represented by a node of tree will, for biochemical reasons, be prone to failure or prone to producing non-specific reaction products.

Each of the trees represents a series of operations that will be performed using tangible hardware and prepared biological reagents. The different trees represent different ordered combinations of steps for making the same desired polynucleotide sequences. Some trees may include operations that, if executed, would perform poorly. For example, a sub-polynucleotide AB could be made by joining together one of two pairs that include A being joined to B′ or A′ being joined to B, where either combination results in AB. However, the difference between A and B′ compared to A′ and B could be the sticky ends of each, where—for a made up example—a 3′ sticky end of A′ form a GC-rich hairpin that inhibits joining A′ to B. In this example, attempting to join A′ to B is prone to failure. Here, it would be preferable to select 211 a tree in which A is joined to B′ over a tree in which A′ is joined to B.

In another example, selecting 211 a good tree can help avoid non-specific or unintended reaction products. For example, there are different combinations of steps for making ABCD from A, B, C, and D.

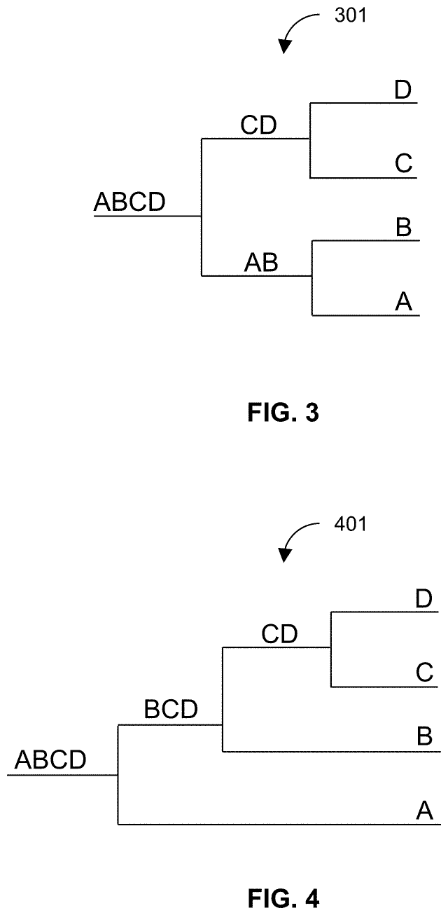

FIG. 3 is a graphical depiction of a symmetrical tree 301 for making ABCD.

FIG. 4 is a graphical depiction of an asymmetrical tree 401 for making ABCD. However, it may be the case that when A and B are mixed, they have a proclivity to form BA as well as AB. In such a case, symmetrical tree 301 will not reliably make ABCD exclusively. However, tree 401 avoids ever combining free A with free B in a manner that exposes a 3′ end of B to a 5′ end of A. In that case, assuming all other joins will happen reliably, then tree 401 will reliably produce ABCD.

The invention provides methods that are useful for automatically selecting tree 401 over tree 301. One suitable approach is to write all of the trees 301, 401 in Newick format, e.g., in a single file. Such a file could include the following lines (among others):
301 ((A,B),(C,D))
401 ((A,(B,(C,D))
501 (A,((B,C),D))

In such a file, the first line is the Newick representation of tree 301 and the second line is a Newick representing of tree 401. A system can progress through the file and assign a score to each tree. The score can be assigned by using a priori knowledge of sequence liabilities or probabilities of success. Such information can be calculated and stored in a suitable data structure such as a matrix or variable structure (an array of hashes is well-suited, where the 5′ oligo of a ligation is an index to an array entry that points to a hash specific to that oligo; in the hash specific to that 5′ oligo, the 3′ oligo of the ligation is key for which the paired value is a ligation score). Scores can represent a probability of successfully making exclusively the intended reaction product at any given node in the tree (noting that a node represents a pairwise combination of sub-parts). Because an oligo has distinct 5′ and 3′ ends, the scores can be represented in an asymmetric matrix. For example, if the scores range from 0 to 1, and it is known that mixing B with A forms BA, and not AB, then the score for combining A and B to form AB can be set to zero.

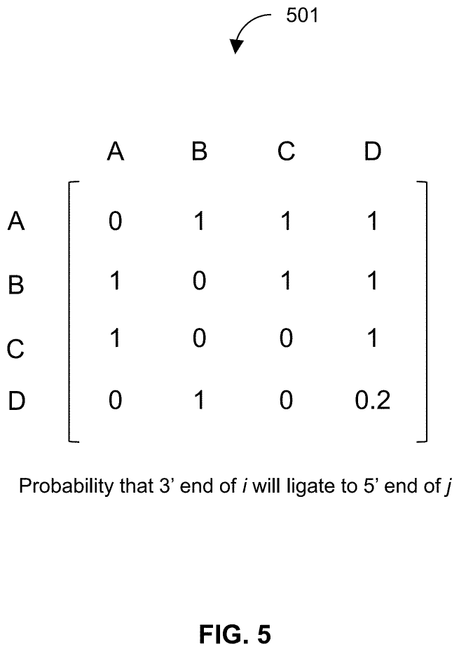

FIG. 5 shows a matrix M 501 that gives a score for the probability that a 3′ end of the ith entry will attach to a 5′ end of the jth entry. Matrix 501 is not empirical and is drawn only to illustrate that when B and A are mixed, they will form AB as well as BA, whereas if C and B are mixed, they will form only BC, and no CB. Other encodings and examples are within the scope of the invention. The matrix 501 is one illustrative approach. The matrix 501 is information rich in that it also shows, for example, that mixing C with D will form CD but not DC. The matrix 501 shows that A, B, and C will not self-polymerase, but reveals that D has a modest probability of attaching to itself. A computer system can automatically apply the matrix 501 to the Newick formatted version of trees 301 and 401. For example, for each pair in the Newick tree, the computer system can calculate a probability that the indicated product will form. In the first row, the entry “(A,B)” indicates that an intended product is AB when A and B are mixed. The computer can read the probability that a 3′ end of B will join to a 5′ end of A and score the (A,B) entry as zero, because mixing A and B has a good probability of making an unwanted reaction product. The score for the tree 301 can be calculated by multiplying together all scores for that row. In such a case, the row of scores for the Newick tree for 301 will include a zero for the “(A,B)” entry, and the entire row will multiply to zero. The computer system has scored tree 301 as 0.

Tree 401, in Newick format, shows that A will be joined to BCD. The computer program looks up the probability of a 3′ end of A joining a 5′ end of B (1) and the probability of a 3′ end of D joining a 5′ end of A (0). That entry in row is scores as 1 (because the Matrix shows no probability of making unwanted product BCDA). In a hypothetical example, the computer system has scored tree 401 as 1.

An important insight here is that there can be arbitrarily large number of trees (e.g., multi-millions). The computer system can write each and every one into a tree file, although real-world situations will present heuristics allowing trees to be ignored. The matrix may be obtained empirically. For example, the matrix may be obtained by some combination of laboratory test results and thermodynamic predictions made in silico. The computer system can apply the matrix to score each tree. The in silico activity of scoring the trees can be parallelized. E.g., half the trees can be written to one file sent to one processor or one node of a Beowulf cluster, while another half are sent, in another file, to another processor. Interestingly, the process of scoring tree does not necessarily need to save or record all scores. The process can simply hold the last calculated score in a variable while scoring a next tree, then compare the newly-calculated score to the last-calculated scores and keep only the better score. Of course, it may be preferable to write every score to a log.

Significantly, the disclosed process scales up arbitrarily. The matrix 501 represents joining together of 4 oligonucleotides. Systems and methods of the invention may be used to create desired polynucleotide sequences on the order of 100,000 bases in length or longer.

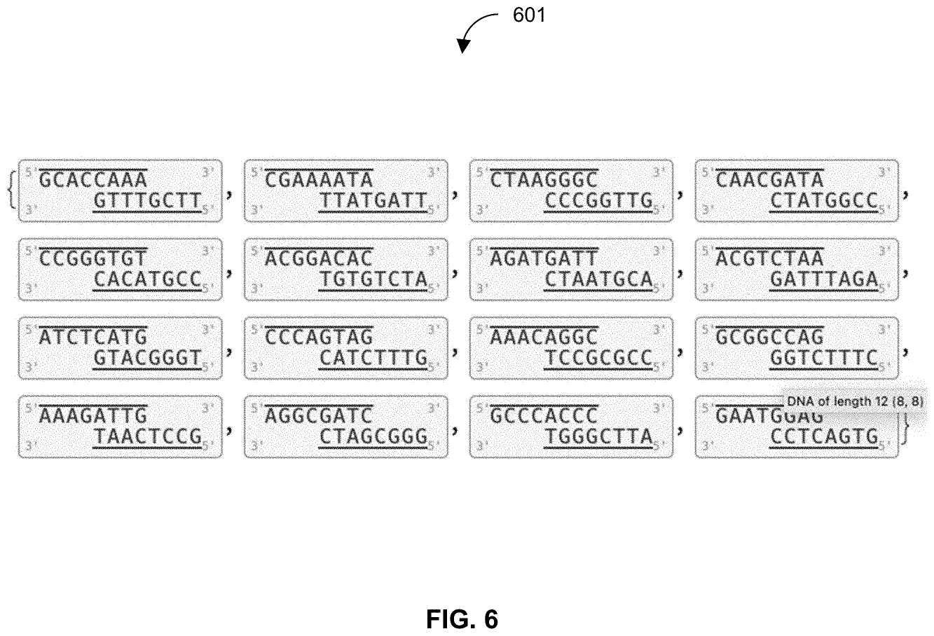

FIGS. 6-10 are used to illustrate a particular embodiment of creating a scoring matrix that is useful in both determining a partitioning of a desired polynucleotide sequence and in selecting a final assembly tree.

FIG. 6 shows a plurality of partially ds oligos that may be used in an assembly described herein. As shown each covalently complete molecule is 8 bases long and they overlap by 4 bases. There are 16 depicted oligos. After the partitioning step, such oligos may be provided in a compartment in the lab. They may be synthesized based on the result of the partitioning or a lab may have a sufficiently large library and, after partitioning, appropriate library plates may be pulled for use. For example, each oligo may be provided in the well of a multiwell plate, where each well includes, e.g., millions of clonal copies of the depicted oligo. A long, desired polynucleotide sequence may be made by ligating the oligos together in a pairwise manner into longer and longer sub-polynucleotides. Method of the invention partition the desired sequence to identify the oligos to use and also predict the success of creating the desired polynucleotide sequence when ligating the oligos together in a pairwise manner. The methods may include (i) partitioning the desired sequence into a set of oligo sequence; assigning scores showing probability of successfully ligating together a pair of oligos (i.e., a positive score to show the probability of obtaining the intended results), and (ii) identifying pairs of oligos with overhangs that can mis-ligate (i.e., a negative score showing a risk of obtaining something other than an intended result).

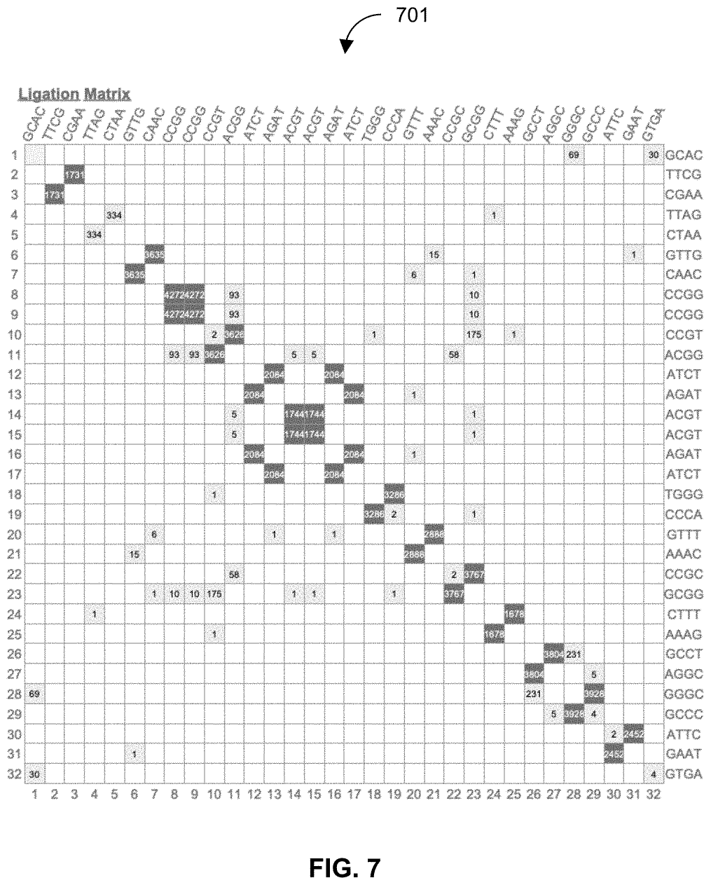

FIG. 7 shows a ligation matrix 701 that gives a score representing a probability that two overhangs will successfully ligate together. The matrix as a whole represents the scoring of each pair of overhangs of a sequence. A high score means high probability of successful ligation. Note that row/column labels appear duplicated because the matrix covers each pair of overhangs proposed to be ligated together in creating one sequence. Even positions refer to a 5′ lag strand and odd positions refer to a 5′ lead strand.

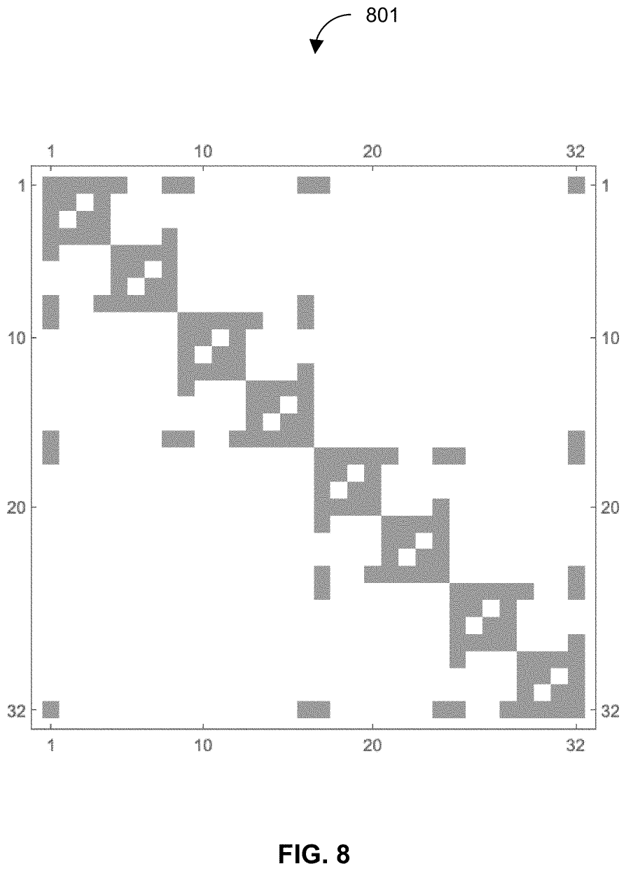

FIG. 8 shows a mapping matrix 801, or partitioning matrix, in which shaded blocks represent any pair of overhangs that can mis-ligate at any point during the assembly process, if a completely symmetric assembly tree is assumed. The mapping matrix 801 is specific to an assembly tree and a partitioning and it shades out blocks where an oligo may mis-ligate to an unintended oligo to form something other than an intended result. The mapping matrix provides information used in selecting a partitioning.

An important part of partitioning is that a computer system separates the desired polynucleotide sequence into constituent oligo sequences with specified sticky ends, in silico. The computer system may iteratively propose multiple (e.g., dozens, hundreds, hundreds of thousands) of partitions and, for each one, perform a tree search process. The mapping matrix is used to score trees for pairs of overhangs that may mis-ligate, i.e., disfavored ligations. One example of a disfavored ligation and how it may be solved by partitioning and tree search is self-ligation. If a partitioning proposes (e.g., when generated automatically by programming logic of the computer system) an oligo with self-complementary sticky ends, the mapping matrix will penalize that oligo for liability for attaching to itself. The computer system can iterate the partitioning by adjusting one sticky end of that oligo by shortening or lengthening that sticky end by one base (and the by two, three, etc.) The mapping matrix encodes the biochemical likelihood of annealing between sticky ends. With a sticky end lengthened or shortened by one or two bases, the mapping matrix (when applied to the assembly tree) may score that portioning in way what permits it be used. The portioning is updated or ascended, meaning that the oligos with the sticky ends indicted by that partitioning are directed to be used. From that in silico direction, the indicated oligos may be synthesized or pulled from a library for use.

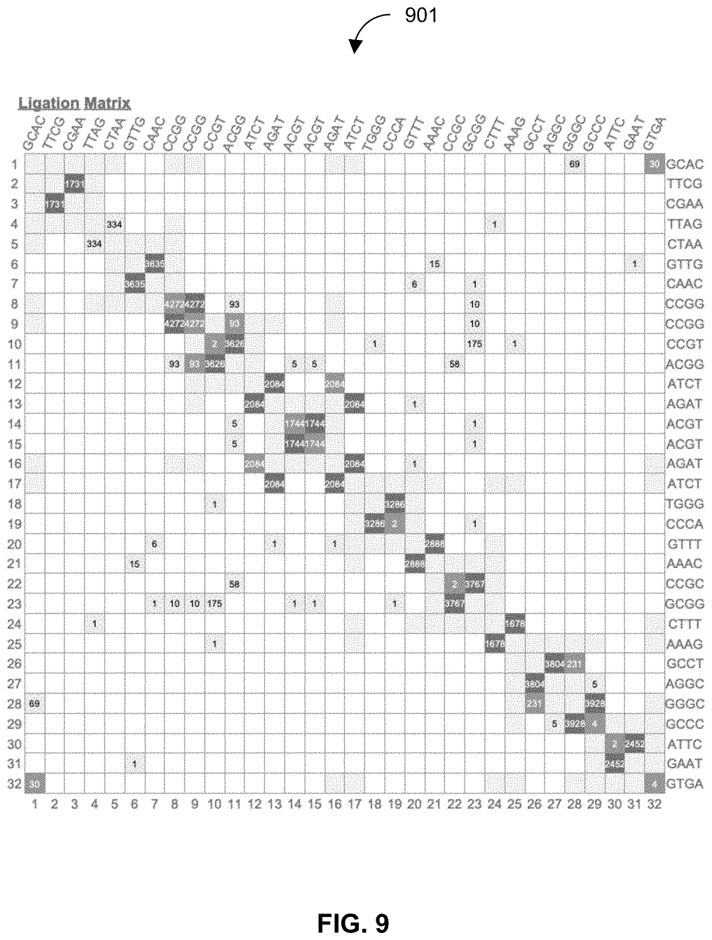

FIG. 9 shows a scoring matrix 901 that results from combining the ligation matrix 701 and the mapping matrix 801. Combining the ligation matrix 701 and the mapping matrix 801 gives an overall score (for example the negative of the sum of all the shaded scores in this representation). For a given assembly tree, the scoring matrix 901 is used to score the tree. Because the trees can be represented as a linear character string in a text format, a computer software module can read along the tree, scoring each element, building up a final score for that tree. By such a process, each tree will have its own score. Note that in these embodiments, a tree represents both a proposed partitioning of the desired sequence into constituent oligo sequence with specified sticky ends and steps for assembling the desired polynucleotide on liquid handling systems. Selecting a tree can thus both guide what set of oligos to provide and operations of the assembly equipment.

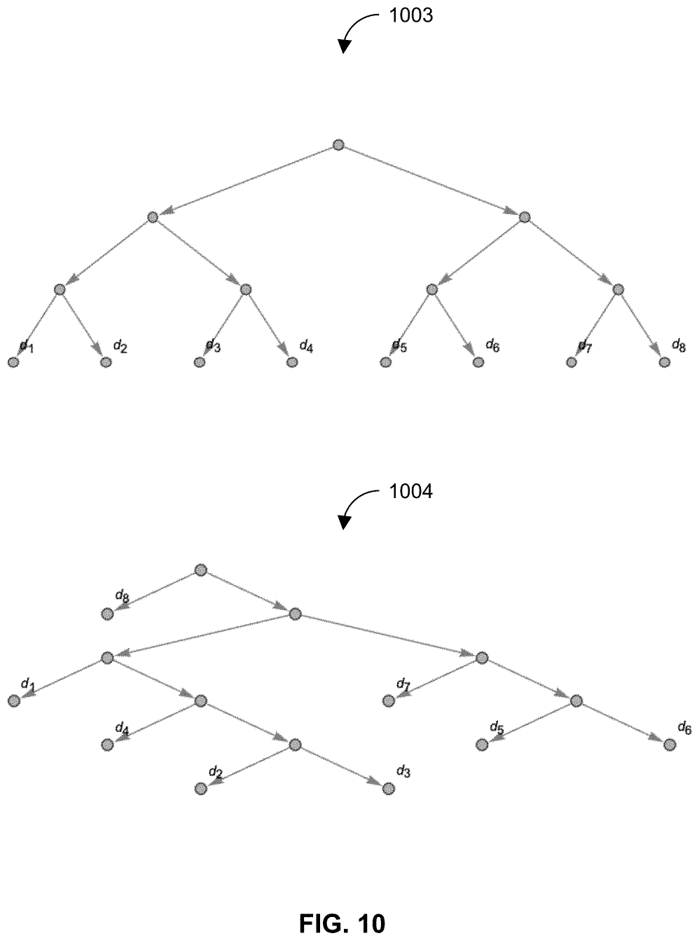

FIG. 10 shows a symmetrical assembly tree 1003 and an asymmetrical assembly tree 1004. It is important to note that (i) there will typically be millions of the trees, and (ii) the trees may be stored in a plain text file, e.g., in the Newick format. That is, the symmetrical assembly tree 1003 and the asymmetrical assembly tree 1004 may be among hundreds, thousands, or millions, or more that are generated. Scoring each trees using its scoring matrix 901 reveals that the symmetrical assembly tree 1003 has a number of sequence liabilities, meaning that the desired nucleotide sequence will be difficult to synthesize on the laboratory instruments. However, the depicted asymmetrical assembly tree 1004 gets a good score. Using the labels from the tree 1004, this suggests that d2 should be attached to d3 and the resultant product should be attached to d4 (steps not found in tree 1003).

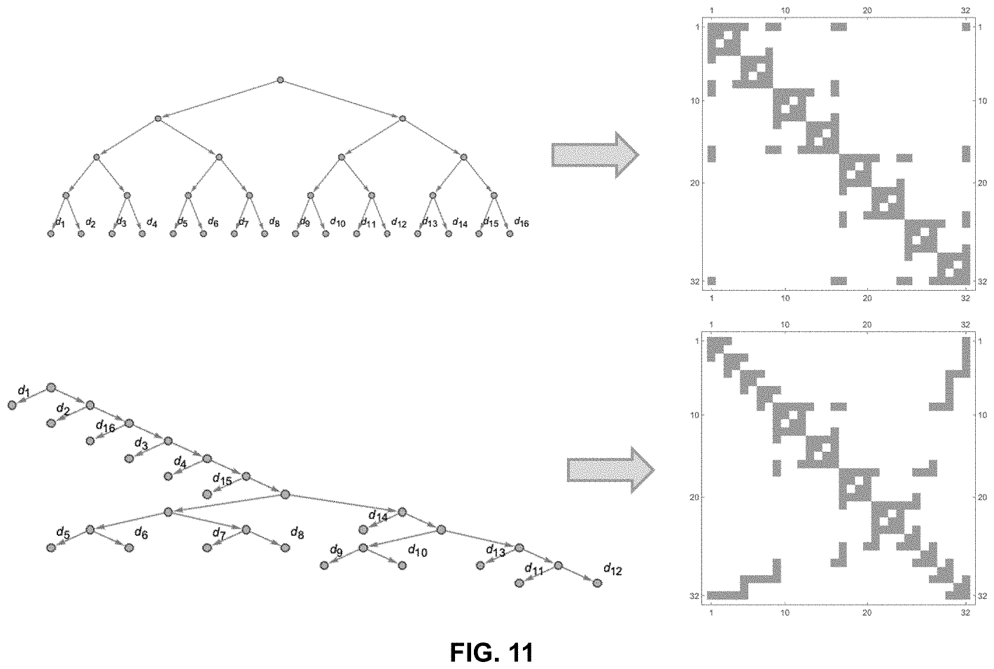

FIG. 11 shows that different assembly trees will have different corresponding mapping matrices. FIG. 10 shows the use of the scoring matrix 901 for assembly tree optimization. The trees shown on the left are the proposed trees, pre-scoring. From those, the mapping matrices are generated, which are then used to score the trees. The different trees represent different ways of combining the components, by changing the order of the ligation reactions. Thus, methods of the invention optimize the assembly tree by scoring all possible trees, which is done by combining the mapping matrices and the partition matrices, and then by picking one tree with a suitable score. In a simple case, the suitable score may be the highest numerical score, but other trees could be selected (e.g., a second-highest, or nearly-highest, or top ten percent, or good enough, or top quartile) for a variety of reasons. For example, one may select a tree in which a number of monophyletic group consist of 8 terminal tax, because those can progress into assembly 225 using an entire width of a 96-well plate.

It is noted that some generated tree structures may not be “good trees”, i.e., may not represent an ordered combination of branches that would results in the desired polynucleotide. However, those “bad trees” need not impede the selection 211 of a suitable tree. In fact, part of the selecting 211 logic in the computer system can include selecting for trees that make the desired polynucleotide sequence. That is, it may not matter what bad trees are among the tree file(s) as those may simply not be selected. This approach admits of one strategy to tree generation and scoring, as it allows trees to be generated quasi-indiscriminately. Trees can be generated without respect to the product that they represent and selection for the product itself can be included in selection for probability of successfully making that product with the laboratory instruments and reagents. This note will have particular applicability in Bayesian tree search embodiments, which allow the generation of a one or a limited number starting trees, making random changes to the starting trees in the style of a Markov chain, evaluating the changed tree versus the previous one and repeating the changes and evaluation from the better of those two (a process that can be parallelized and used to cover greater tree space by periodically swapping parts of trees between chains being processed in parallel). Regardless of the particular tree search algorithm used, preferably the selecting step 211 comprises calculating a score for each of a plurality of trees. The calculated score represents a measure of success that the tree structure can be used to successfully produce a molecule with the desired polynucleotide sequence. The success measure can be a predictor of (e.g., correlation with) any relevant factor in producing a product including, for example, success or failure, but also possibly molecular concentration, relative concentration or any other physical-chemical measure correlated with the yield of the assembly.

While a matrix is shown in an embodiment above, the probabilities for each ligation step or associated with each oligo or sub-polynucleotide can be stored in any suitable format or structure. As shown above, the probabilities were applied to tree nodes, to evaluate a probability of ligation success. Oligos or sub-polynucleotides (as compared to the action of joining those) can also be scored. For example, the scores can include scores to represent the risk that a stretch of bases include a problematic restriction site, an extreme GC values, a run of CpG repeats, a risk of thymine dimers, etc. The scores may represent probabilities of success in assembling partial constructs and/or stored probabilities of mis-assemblies.

As discussed, scores are assigned to a plurality of assembly trees. Preferably, a computer system selects 211 a tree structure that provides an optimal combination of branches. While the scores may be numerical, it is important to note that it is not necessary or required to select “the” optimal tree. Millions of trees can be scored, in an example, from 0 to 1. It may be suitable to simply select a tree with a score greater than 0.9. For example, they system may score trees until several (e.g., 15) trees have obtained scores greater than 0.90 and then select the one tree of those with the best score, and proceed to directing assembly of the desired polynucleotide sequence (even if millions of the tree have not yet been scored). Similarly, the system may output a number (e.g., hundreds) of top-scoring trees, including one with a highest-numerical value. But the highest-scoring tree may not be used. For example, human review may reveal liabilities. Or, a wrapper script for the liquid handling instruments may include data about the instruments and select a tree with certain characteristics that are specifically suited to the liquid handling instruments (e.g., numerous monophyletic blocks with 96 terminal clades, because the liquid handling instruments can build from those efficiently with 96-well plates). Thus, for example, in some embodiments, computer system of the invention search tree space to identify local maxima and either algorithmically select one tree from one of those, or push a number of trees (e.g., 10) out for human review or for evaluation by a laboratory instrument software wrapper.

Other strategies for selecting 211 an assembly tree are within the scope of the invention. For example, a computer system may execute a machine learning algorithm to select a tree with an optimal combination of branches for assembling 215 the molecule. An advantage of using a machine learning algorithm is that, optionally, with a machine learning system, one need not provide a matrix of scores for nodes (and/or branches) of a tree. One may use such scores during a selecting 211 step. However, those scores may be used as part of training data when training a machine learning system. Moreover, it is not necessary to use such scores. For example, one can train a machine learning system using tree files, output polynucleotides, and success metrics as training data. A machine learning system can be trained with training data that includes, for each of a plurality of polynucleotides that was made, a tree used in making the polynucleotide, optionally one or more alternative trees that were not used, optionally the sequence of the polynucleotide (optional in part because that information is inherent the tree), and success metrics, such as time to make, cost to make, failure count, cost of wasted reagents, etc. Interestingly, one may use a machine learning algorithm to generate the matrix of scores that could optionally then be used in a non-machine learning approach to tree selection. Using machine learning to generate the score matrix is appealing because it should work for smaller training data sets but generalize up to large data sets. One could train the machine learning system on trees, oligos, and score matrices, where each tree has, e.g., 12 terminal taxa and 12 oligos (plus their sticky ends) are given. After such training, the machine learning system is given a test input comprising 96 oligos (and optionally one or more trees). The trained machine learning system can then output scoring matrices for trees with 96 terminal taxa. Whether the machine learning system is used to generate the score matrices, to select a tree, both, any suitable machine learning system may be used. Suitable machine learning systems may include one or more of a decision tree algorithm, a random forest algorithm, an extreme gradient boosting algorithm, an adaptive boosting algorithm, or a deep learning algorithm such as a deep neural network.

The particular machine learning algorithm maybe selected based on the particular problem. For example, a random forest algorithm is well suited to evaluate text inputs and compare among a large number of entries with comparably formatted text strings. So a random forest algorithm may be used for selecting from a very large number of Newick trees.

In another example, a convolutional neural network (CNN) transforms dimensionality of inputs in feature representation. Because of that, a CNN may perform well with multiple inputs of disparate scale or dimensionality. As such, a CNN may do well when trained on small trees, but used to score much larger trees. In fact, a CNN may perform particularly well at scoring large trees when trained by a multiple instance learning (MIL) model on bags of small trees that are sub-parts of much larger trees. In MIL, each bag typically comes with one score for the whole bag. So training a CNN by MIL may include presenting training data comprising bag of small trees (e.g., 12 terminal taxa), all trees in a bag taken from one large tree (e.g., 96 terminal taxa) and a score representing a success of making the polynucleotide by that bag. An option for training a machine learning algorithm is to train the ML algorithm in the background while performing methods 201 of the invention using non-machine learning programming to make a number of desired polynucleotide sequences over time. For example, one would write a tree-scorer (e.g., in Python) that multiplies Newick tree elements by matrix scores and then use that system to make long (>100 kb) polynucleotides for a period of time while passing bags of data to a CNN for MIL training. After the period of time, the CNN may have become trained and may be able to step in and score and select trees.

Once the tree is selected 211, the method 201 includes assembling 215 the desired polynucleotide sequence. Any suitable tools, reagents, or instruments may be used for making the polynucleotides. Preferably, the reagents include oligos provided and stored in some sort of vessel or compartment. In some embodiments, the oligos are provided within wells of a multiwell plate. In other embodiments, the oligos are each provided in a tube such as a micro-centrifuge tube. The oligos may be in an aqueous solution or suspension, or optionally provided in hydrogel beads or a matrix. The oligos maybe dried, e.g., lyophilized, and resuspended or dissolved at the assembly 215 step. In some embodiments, a microfluidic apparatus is used in the assembly step, in which the microfluidic apparatus handles droplets (e.g., aqueous droplets surrounded by an immiscible phase) that contain portions of the desired polynucleotide and optionally reagents for assembling the desired polynucleotide.

Assembly 215 may be accomplished by directing a robotic fluid handling device to make the desired polynucleotide.

The invention has various important features that may form important parts of specific embodiments. For example, in different use cases, methods of the invention are suited to different applications such as make one specific large scale polynucleotide with a specified sequence or for making variant libraries. In another example, recursive assembly aids in keeping assembly methods of the invention practicable when making very long molecules. In another example, methods of the invention map assembly trees to instruments in a manner that may customize the tree or customize the instrument instructions in a manner specific to the laboratory instruments such as liquid handling systems.

### Use Cases

The disclosure is applicable to multiple use cases in which a desired biopolymer is to be synthesized.

A first such use case is genome-scale synthesis. At the core of genome-scale synthesis is receiving an order describing one genomic-scale polynucleotide, e.g., a desired polynucleotide sequence at a length significantly greater than 10 kb, typically greater than 100 kb. Methods include providing a plurality of oligos from which the genomic-scale polynucleotide will be made; generating a plurality of assembly trees each describing an ordered set operations for attaching together the oligos to form the genomic-scale polynucleotides; scoring the trees to give each tree an assembly score, a score representing a predicted measure of success that the tree will result in the molecule with the desired polynucleotide sequence; selecting a tree having a score that meets a threshold; and directing laboratory instruments to make the polynucleotide as represented by the tree.

For genome-scale synthesis, recursive assembly will likely be used. Discussed in greater detail below, recursive assembly includes selecting a suitable sub-tree for a subset of the oligos and treating that sub-tree as a terminal taxon in selecting a higher-level tree that includes the sub-tree. Recursion may be multiple, with multiple sub-trees identified that will be used at a parallel level of a higher tree or with multiple sub-trees wat will be nested one in another in a higher tree. Of particular importance in genome-scale synthesis is the provisional of one of, or a specific number or quantity of clonal copies of, the desired polynucleotide sequence.

Another use case for methods of the invention is the provisional of a library of variants. Commonly, in such a case, variants refer to a number of molecules that have numerous elements in common and that also have numerous differences among them. One example of a library of variants includes expression vectors for a gene or operon in which, among the variants, coding sequences and/or regulatory elements (e.g., promotors and repressors) are rearranged, duplicated, or omitted. A user may wish to have dozens, hundreds, thousands, or more such variants that include, for example, open reading frames for a protein of interest but a variety of arrangements of regulatory elements, exons, or both (e.g., omitting certain exons can promote expression by the expression vector of certain splice-variants of issue). A library of variants may be used to express large variety of proteins such as antibodies or enzymes and the library may, in turn, be useful tool for screening for, e.g., antibody binding, enzyme efficiency, etc., and may be useful in metabolic engineering.

### Recursive Assembly

As a practical matter, tree selection may need to be approached heuristically. For example, if a 100 kb sequence is to be assembled from 8 mers, there may be greater than 10\hat{ }50 possible assembly trees. Available computing power may not admit of generating and scoring all the assembly trees. One available heuristic is to break down the task of the tree generation/selection into small parts and perform methods 201 of the disclosure on the smaller parts to generate subtrees and then connect the subtrees together into the selected assembly tree. Interestingly, recursive tree selection in silico may provide efficiencies when the molecule is synthesized. Each sub-tree of a recursive tree may represent a sub-segment of the desired polynucleotide that can be made with one “run” of a robotic handler or one plate of reagents, so the recursive tree (even though recursive sub-trees are a product of heuristics to improve in silico tree search algorithms) may lend itself to modularity on the equipment that minimizes plate swaps or improves parallelization.

To illustrate one example of recursive assembly, one may take groups of some number (e.g., 12) of contiguous oligos found together in the desired sequence and perform the method 201 for those 12 oligos to select one subtree. That subtree can then be used recursively as one terminal taxon in selecting a higher-level tree.

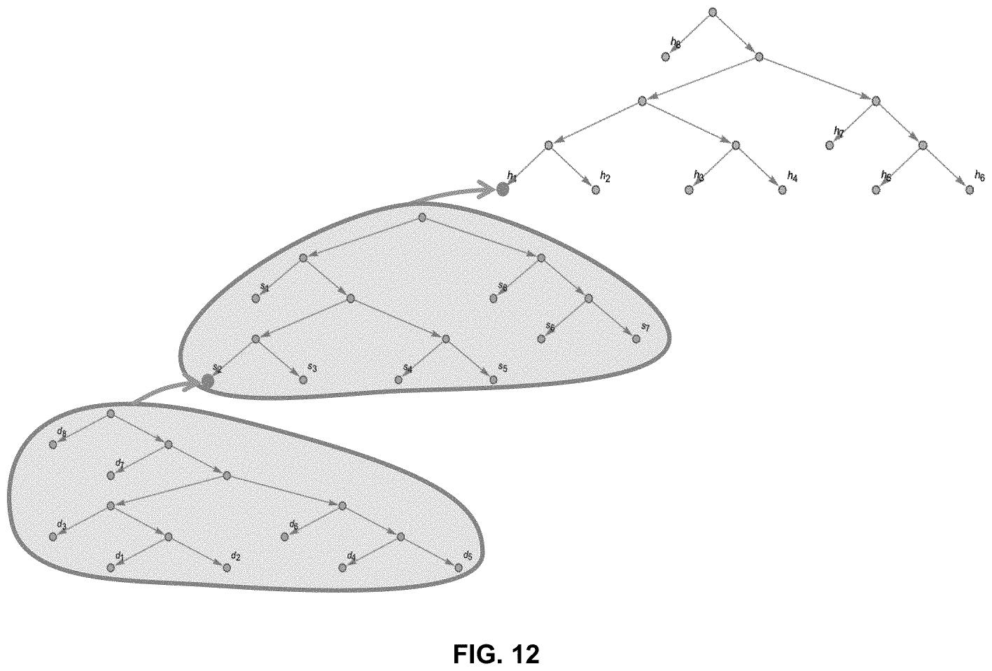

FIG. 12 illustrates recursive optimization. Given the combinatoric high number of possible trees, the partitioning is divided into subgroups and each subgroup is optimized independently. Each subgroup is reduced to a “leaf” at a higher level of the tree and the process is repeated. The figure shows two levels of recursion, with a subgroup forming a subtree that is used as a leaf in a higher-level tree. The method may include joining subgroups of branches together to form a branch path comprising a desired polynucleotide sequence. Oligos may typically be between about 8 and 40 nucleotides in length (commonly with single stranded sticky ends and a double-stranded middle segment). Without recursion, there may be greater than 10\hat{ }50 or more assembly trees that would otherwise need to be created which maybe computationally intractable for some computing resources. By performing recursive assembly, a fully optimized tree may be identified even for arbitrarily large (e.g., genome-scale) polynucleotides.

FIG. 13 illustrates a fully optimized tree that results from recursive assembly. The disclosed method provides for recursively optimizing the full assembly tree without excessive need of computational resources. As a positive side effect, the tree remains shallow enough to allow a high level of parallelization during automated assembly.

### Mapping Trees to Instruments

In all instances, the disclosure relates to operating laboratory instruments to make synthetic biopolymers. While different laboratory instruments may be used, including cherry pickers, robot liquid handlers, and acoustic liquid handlers, the processes share features of combining short oligos to make (preferably) covalently contiguous, very long (e.g., genome scale) polynucleotides. Such instruments commonly work with standardized multiwell plates or arrays, or similarly with strips or racks of tubes, having standardized dimensions or numbers. Once an assembly tree is selected, the tree is mapped to the laboratory instruments.

In one exemplary embodiment, a computer system that issues directions to a liquid handling system parses the assembly tree into monophyletic groups that have the largest number of terminal taxa without exceeding a number inherent in a laboratory consumable, while also trying to minimize the number of such groups. For example, the liquid handling system may operate with 384-well plates. The computer system may include a wrapper script that maps the tree to instructions recognized by the liquid handling system. The wrapper script can receive the number 384 as a flag (“−384”) when given the selected tree file as input. The wrapper script can then identify groups within the tree in a greedy fashion, trying to identify the largest contiguous (i.e., monophyletic) sub-trees that have no more than 384 members. Then the wrapper script can take each such subtree and issue pipetting or transfer commands to the liquid handling system. For example, if the subtree includes (D,Q)(P,N) . . . and the wells are referenced accordingly, the wrapper script can issue a command to transfer 5 nL from source well D and 5 nL from source well Q into a destination well 1 while transferring 5 nL from source well P and 5 nL from source well N into a destination well 2. At a subsequent step, the destination wells can become source wells, and the wrapper script can issue commands to mix ingredients from those. By such an algorithm, the wrapper script maps the assembly tree to the liquid handling system.

Other examples and embodiments are within the scope of the disclosure. For example, a liquid handling system may operate with 384-well plates. The system may have a number (e.g., 17) jobs pending. The wrapper script may scan the jobs to look for jobs that can be accomplished entirely from the 192 wells that constitute half of a 384-well plate. The script may identify that jobs 2 and 11 are such jobs, and may initiate those jobs to be run simultaneously from shared 384-well plates.

Library-of-variant use cases are particularly conducive to benefiting from an intelligent mapping of assembly tree to liquid handling system. In common variant case, a large number of variants will have large segments in common, albeit at different positions along the final variant molecules. The script can recognize those common segments and direct them to be made early and in excess and can park those segments in their own containers (e.g., wells), then upon making each variant, the system can treat the segment as an oligo (which is, not coincidentally also an implementation of recursive assembly).

In yet another example of mapping a tree to instruments, when using a liquid handling robot with a multi-channel pipette, the system can enforce an all-or-non rule for rows or columns of a plate so that, as the robot passes over a plate, a pipette is either (i) used in every well of that row, or (ii) not used at all. While this may lead to certain apparent inefficiencies where, for example, 94 oligos are going to be joined, the system only uses 88 wells from a 96 well plate but then uses a new plate for the remaining 6 oligos, this can have benefits in terms of avoiding cross-contamination.

Systems and methods of the invention are executed using laboratory instruments under control of operational systems.

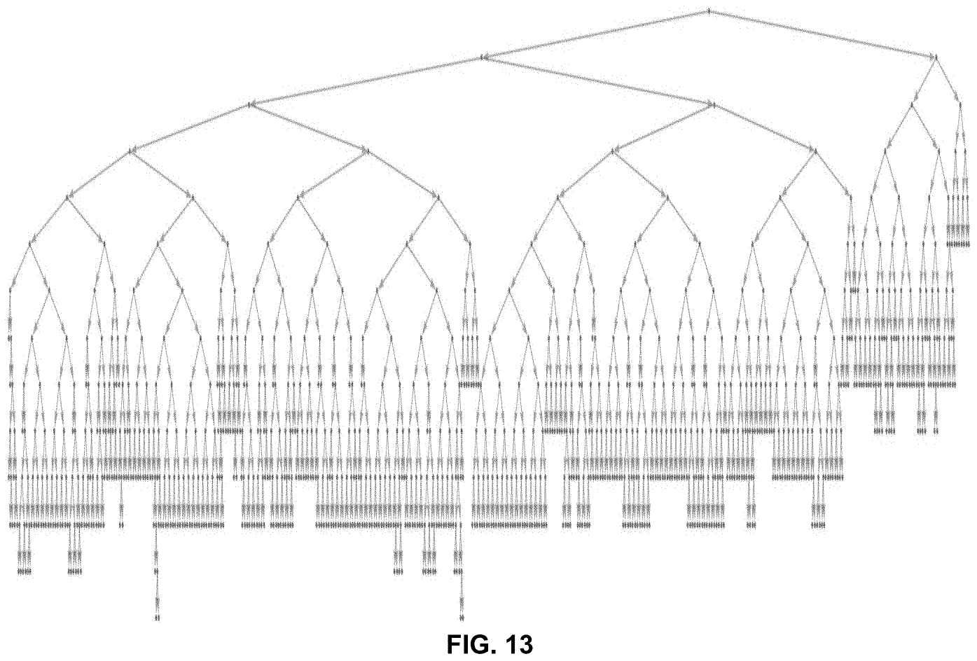

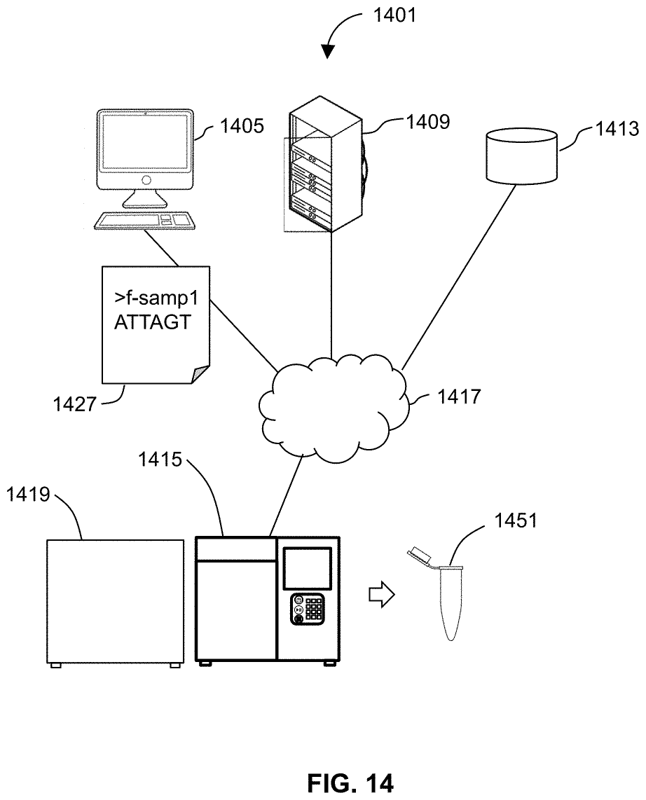

FIG. 14 illustrates components that may be included in a system 1401 of the invention. Typically, at least one liquid handling system 1415 will be under control of at least one computer system 1409. The system 1401 receives a desired polynucleotide sequence 1427 (e.g., emailed in from a client computer 1405, optionally in FASTA format). The client computer 1405, the computer system 1409 and the liquid handling system 1417 preferably communicate via a communications network 1417 which may include any combination of local network hardware, the Internet, and cellular communication networks. The system may preferably have access to storage 1413. Either or both of the computing system 1409 and the storable 1413 may be provided by one or a plurality of computers, servers, or cloud computing resources (e.g., Amazon Web Services (AWS) on-demand server computers). The storage 1413 may include information on a plurality of oligos that are provided within laboratory equipment 1419, which is preferably operably coupled to liquid handling system 1415. For example, the laboratory equipment 1419 could be a robotic liquid handler with a plurality of multiwell plates housed therein, and wells of the plates could each contain a large number (millions) of clonal copies of oligos. The sequences of those oligos and their locations within the plates may be stored in the storage 1413. Upon receipt of the desired polynucleotide sequence 1417, the computer system 1409 performs the method 201 to select 211 an assembly tree. Optionally, the method includes partitioning the desired polynucleotide sequence 1417 to identify the oligos to use and using laboratory equipment to create or provide the identified oligos. The system 1401 directs assembly of the polynucleotide 1451 by the liquid handling system 1415 according to the selected tree. The method produces a new synthetic molecule, the polynucleotide 1451, which may be provided in a suitable vessel or tube, such in a microcentrifuge tube in solution or in another format such as dried (e.g., lyophilized) or embedded in a matrix, e.g., within a hydrogel bead, optionally frozen (e.g., at −80 degrees C.) for long term (>30 d) storage. The polynucleotide 1451 in its tube may optionally be packed (e.g., in dry ice) and shipped.

In the system 1401, the computer 1409 is operable to receive the desired polynucleotide sequence, generate a plurality of trees, each tree giving an order of attachments among oligos 601 to form the polynucleotide; select one of the trees having an optimal production score; and make the polynucleotide 1451 by joining the oligos 601 in the order given by the selected tree 1004. Preferably, leaves of the trees represent the oligos and nodes of the trees represent attachments between oligos. But it is important to note that those can be reversed, and a purported system that stores oligos in nodes and represents ligations in edges is typically a sematic variant that is equivalent in all functions. The system 1409 selects 211 the tree by calculating, for each tree, a score representing a probability that the nucleic acid will be successful made using that tree. Preferably the tree is scored using (i) a matrix 701 of stored probabilities of success in ligating overhangs of the oligos; (ii) a matrix 801 of stored estimates of risk of mis-ligations between unintended pairs of the oligos; or (iii) both.

As shown in FIG. 12, the generating and selecting steps may be performed for a first subset of the oligos to form an intermediate tree; and the intermediate tree may be used as a leaf in selecting a final tree having the optimal production score. Recursive versions of the methods may include identifying subgroups each comprising a computationally tractable number of oligos (e.g., fewer than about twenty to one hundred of the oligos); generating, scoring, and selecting sub-trees for each subgroup; and joining together one optimal scoring sub-tree from each subgroup to yield the selected tree having the optimal production score. Within the computer system 1409, a software package may generate all possible rooted bifurcating trees; store all of the trees in memory; apply a scoring matrix to each of the trees in memory to score all of the trees; and select a best-scoring tree for the sub-group. The computer system 1409 may use a software package to transform the selected tree into instructions executed by a liquid handling system 1415 to transfer the oligos 601 among storage vessels comprising reaction vessels optionally with reagents such as ligase, in the order given by the selected tree to make the polynucleotide 1451. In mapping-tree-to-instruments embodiments, the computer system 1409 may create a description of an arrangement of the oligos in a multi-well plate, wherein the described arrangement minimizes steps or operation time of a liquid handling system 1415 making the polynucleotide 1451 by joining the oligos 601 in the order given by the selected tree 1004. Preferably, the selected tree is asymmetrical (a first leaf is connected to a root of the selected tree by a first number of nodes and edges and a second leaf is connected to the root of the selected tree by a second number not equal to the first). The computing system 1409 may perform the selecting 211 step in a manner biased to favor shallow trees over deep trees, because shallow trees maximize parallelization, which minimizes time to make the polynucleotide 1451.

The invention provides methods for synthesizing large biopolymers such as genome-scale polynucleotide molecules. Where genome-scale polynucleotides are to be made by attaching together a number of oligos, assembly of the desired polynucleotide from the oligos is represented as a tree, in which the branches represent nucleotide sequences and the nodes represent attachments between pairs of oligos. Numerous assembly trees are each generated and scored in silico according to how successful that tree will be in directing assembly of the desired polynucleotide, given biochemical properties of the oligos and proposed ligations. Systems and methods of the invention select a suitable tree and operate liquid handling systems to perform operations as shown by the selected tree to make the desired polynucleotide.

Embodiments may use microfluidic handlers that are capable of transferring serially or in parallel the full or partial contents of one or several compartments into other pre-specified compartments that may or may not be empty. Reaction products may be purified e.g., by gel electrophoresis, hybrid capture with biotinylated probes, chromatographic, or affinity separation methods. Assembly method preferably include connecting oligos by a ligation reaction which comprises enzymatic, chemical, or an adaptor ligation. Some embodiments use a ligase, such as any one of a T3, T4 or T7 DNA ligase, or a RNA ligase, a polymerase or ribozymes. Preferably T4 DNA ligase, T7 DNA Ligase, T3 DNA Ligase, Taq DNA Ligase, DNA polymerase, or engineered enzymes are used. Preferably, the following ligation reaction is used: T4 DNA Ligase, at a concentration of 10 cohesive end units per μL supplemented with 1 mM ATP (Sambrook and Russel, 2014, Chapter 1, Protocol 17). Assembly may include hybridizing matching overhangs of a ds oligo, or hybridizing a suitable ss oligo linker. A solid carrier may be used to immobilize one or more of said oligos, the target polynucleotide, or one or more intermediate(s) of assembly. Immobilization may be done with avidin coated beads, by modification of the oligo/poly-nucleotides such as by biotinylation or amino modifications. Modifications may use a surface treated with an amino silane for attachment to 3-aminopropyltrimehtyoxysilane or 3′ glycidoxypropyltrimethoxysilane. Surface chemistries amenable to covalent attachment to nucleic acids include carboxylic acid, an aliphatic amine, aromatic amine, chloromethyl (vinyl benzyl chloride), amide, hydrazide, aldehyde, hydroxyl, thiol, or epoxy, among others. Immobilization/attachment to a solid support may use any of the chemistries described in “Strategies for attaching oligonucleotides to solid supports” by Integrated DNA Technologies, 2014 (22 pages), incorporated by reference.

Oligos may be ordered, provided from a library, or produced by a suitable method, such as chemical polynucleotide (or oligonucleotide) synthesis methods, including the H-phosphonate, phosphodiester, phosphotriester or phosphite triester synthesis methods, or any of the massively parallel oligonucleotide synthesis methods e.g., microarray or microfluidics-based oligonucleotide synthesis. The oligos can be produced by any of the enzymatic polynucleotide (or oligonucleotide) synthesis methods e.g., ssDNA synthesis by DNA polymerase proteins or by reverse transcriptase proteins, which produce hybrid RNA-ssDNA molecules. Specifically, the enzymatic polynucleotide synthesis reaction is performed in vitro. The synthesis reaction may be performed to produce RNA, DNA, xeno nucleic acid (XNA) (which may generally include 1,5-anhydrohexitol nucleic acid (HNA), Cyclohexene nucleic acid (CeNA), Threose nucleic acid (TNA), Glycol nucleic acid (GNA), Locked nucleic acid aka bridged nucleic acid (LNA), Peptide nucleic acid (PNA), Fluoro Arabino nucleic acid (FANA)), or hybrids or any combinations of the foregoing.

Oligos may be modified by any one or more of phosphorylation, methylation, biotinylation, or linkage to a fluorophore or quencher. Oligos may be capped or blocked to prevent attachment/polymerization to additional nucleotides (e.g., until un-blocked). Suitable blocking or capping chemistries may include those discussed in U.S. Pat. No. 10,041,110; WO 2018/152323; WO 2021/058438; WO 2021/213903; and WO 2021/116270, incorporated by reference. The library described herein may comprise library members which are oligos that can be any or all of the following: unmodified ss; phosphorylated ss; methylated ss; biotinylated ss; phosphorylated, biotinylated and methylated ss; unmodified ds; phosphorylated ds; methylated ds; biotinylated and phosphorylated ds; biotinylated and methylated ds. Preferably, library members comprise a 5′-phosphorylation. Specifically, the library described herein comprises ss oligos comprising fluorophores or quenchers and ds oligos comprising fluorophores or quenchers.

Oligos may be provided in a storage-stable form, preferably a form which is storage-stable for at least 6 months at room-temperature. Oligos may be stored in storage containments in a dry state. Dry-state is, for example, achieved by lyophilization, freeze drying, evaporation, crystallization or the like. The enzymes which catalyze the degradation of nucleic acids are typically active at room temperature in a fluid biomolecule preparation. Dry-state storage inhibits such enzymatic activity because such enzymes are generally inactive upon de-hydration and because the degradative chemical reactions which they catalyze typically entail the addition of water (i.e., hydrolysis) of a protein or nucleic acid molecule, thus producing protein or nucleic acid backbone cleavage. In the dry state, there is little or no water (e.g., less than 5%, 4%, 3%, 2% or 1% (w/w) water) as a chemical reactant to support such enzyme catalysis. Additionally, any non-enzymatic hydrolysis of protein or nucleic acid is similarly inhibited, since water is generally unavailable for such reactions.

Oligos may include bases such as “A” denoting deoxyadenosine, “T” denoting deoxythymidine, “G” denoting deoxyguanosine, or “C” denoting deoxycytidine, “U” denoting uracil, or other natural nucleosides (e.g. adenosine, thymidine, guanosine, cytidine, uridine), nucleotide-analogs e.g., inosine and 2′-deoxyinosine and theirs derivatives (e.g. 7′-deaza-2′-deoxyinosine, 2′-deaza-2′-deoxyinosine), azole- (e.g. benzimidazole, indole, 5-fluoroindole) or nitroazole analogues (e.g. 3-nitropyrrol, 5-nitroindol, 5-nitroimidazole, 4-nitropyrazole, 4-nitrobenzimidazole) and their derivatives, acyclic sugar analogues (e.g. those derived from hypoxanthine- or indazole derivatives, 3-nitroimidazole, or imidazole-4,5-dicarboxamide), 5′-triphosphates of universal base analogues (e.g. derived from indole derivatives), isocarbostyril and its derivatives (e.g. methylisocarbostyril, 7-propynylisocarbostyril), hydrogen bonding universal base analogues (e.g. pyrrolopyrimidine), or any of the other chemically modified bases (such as diaminopurine, 5-methylcytosine, isoguanine, 5-methyl-isocytosine, K-2′-deoxyribose, P-2′-deoxyribose). The building blocks are linked by phosphodiester linkage or peptidyl linkages or by phosphorothioate linkages or by any of the other types of nucleotide linkages.

Specifically, the target polynucleotide has a length of at least 100 base pairs (bps). Specifically, said target polynucleotide has a length of at least 150, 1,000, 10,000 or 100,000 bps or even longer can be produced. Methods of the invention may include a finalization step e.g., to add one or more nucleotide(s) which correspond to those previously removed from the 3′-end and 5′-end, respectively, to prepare a template of such target ds polynucleotide for the purpose of assembly of the target ds polynucleotide according to a template sequence, such as e.g., to generate blunt ends. Specifically, one or more oligos may be selected for producing blunt ends, which are complementary to any overhang of a prefinal intermediate polynucleotide i.e., complementary to the sticky ends of the polynucleotide. Specifically, respective oligos can be used as primers in a PCR reaction to amplify the final product and to add the remaining oligos to each strand to synthesize the complete target polynucleotide with blunt ends.

Specifically, said finalization step comprises a purification step of a PCR product that has been produced employing standard kits, such as the Monarch PCR & DNA clean up kit from New England Biolabs (product no. T1030), to eliminate remaining oligos, oligos, enzymes and reagents, thereby obtaining the target ds polynucleotide as a purified DNA product, ready for further use. Methods may include enriching the target polynucleotide or one or more intermediates of assembly, by polymerase chain reaction (PCR). Molecules may be purified by immobilization on a solid phase using a tag, for example a biotin tag, and enrichment using, e.g., PCR amplification. According to a preferred embodiment, two sets of primers are used for target specific enrichment and simultaneous elimination of the tag. Specifically, by using a set of primers specific to the 5′ end of the leading strand and a set of primers specific to the 5′ end of the lagging strand of the polynucleotide that is to be enriched, each comprising a primer that is complementary to at least the overhang and a primer that is complementary to the core sequence of the polynucleotide, the target polynucleotide is amplified without the tag sequence. This has the profound advantage that no additional step is required to remove the tag sequence, e.g., by enzymatic digestion.

Methods may include sequencing the polynucleotide molecule verify the degree of identity with the desired sequence. Any suitable sequencing method may be used such as pyrosequencing, Illumina sequencing, SOLiD sequencing, semiconductor sequencing, DNA nanoball sequencing, Heliscope single molecule sequencing, Single molecule real time (SMRT) sequencing, or Nanopore DNA sequencing. Methods may include restriction or chemical modification e.g., to facilitate cloning the target polynucleotide into a vector or plasmid.

The polynucleotide molecule may be modified by enzymatic modification, employing any one or more of methyltransferases, kinases, CRISPR/Cas9, multiplex automated genome engineering (MAGE) using λ-red recombination, conjugative assembly genome engineering (CAGE), the Argonaute protein family (Ago) or a derivative thereof, zinc-finger nucleases (ZFNs), transcription activator-like effector nucleases (TALENs), meganucleases, tyrosine/serine site-specific recombinases (Tyr/Ser SSRs), hybridizing molecules, sulfurylases, recombinases, nucleases, DNA polymerases, RNA polymerases or TNases.

### EXAMPLES

### Example 1. Determining Optimal Assembly Tree for the Assembly of a Target Molecule of 600 bp with the Optimal Asymmetric Workflow by Ligation of 50-Mers

For this example, a 600 bp random sequence (SEQ ID NO: 1) is chosen as sequence if interest (SOI). The sequence will be first partitioned into 12 segments of 50 bp each and then the optimal assembly tree will be determined by considering the best according to a predefined objective function.

Choosing the Objective Function

First, an objective function was chosen for evaluating a partition by defining a weight to every node n according to whether the oligos at each node can react in a unique manner (w=1) or not (w=0). More specifically, any two oligos have a total of four overhangs (O₁, . . . , O₄) where we want that
O₂ and O₃ match according to Watson-Crick pairing
any other pair of overhangs does not match.

Mathematically, this can be represented by a symmetric adjacency matrix A (Aᵢⱼ=Aⱼᵢ) where a 1 indicates a matching pair and a 0 indicates the pair does not match. The score wₙ at node n is then defined as

$$w = \{ \begin{matrix}0 if {O}_{ij} = 1 for i , j \neq 2 , 3 \\ 1 otherwise\end{matrix}$$

Other metrics can be incorporated based, for example, in relation to ligation activity, on frequency of recovered clones from a controlled experiment with proper design of overhang pairs.

Considering all possible oligo pairings in a partition with N elements, the adjacency matrix for the entire partition will have N×N total elements. To have a successful assembly, only consecutive elements in the partition need to form a proper ligation, whereas the other pairings would form a misligation if corresponding oligos are mixed. The assembly tree score is then obtained by adding all those elements of the matrix that correspond to a proper ligation and subtracting those corresponding to a misligation. Formally, we can consider another two matrices: Lᵢⱼ is the ligation matrix, for which each element is 0 except those corresponding to successive overhangs, which are the ones that must be ligated; Mᵢⱼ is the misligation matrix, which represents the overhangs that will be in contact at any point during the ligation (it is indeed tree-dependent), hence it is 1 for all the pairs of overhangs that are in contact and zero otherwise. The score for a tree is then calculated by the following equation:

$$Score = \frac{1}{2} {\sum }_{i , j} {A}_{i , j} \times ( {L}_{i , j} - {M}_{i , j} )$$

Partitioning the Sequence

SEQ ID NO:1 was first partitioned in a naïve manner, that is in equal lengths of 50 nt and with overhangs of length=4. This results in 24 single stranded oligonucleotide sequences (SEQ IDs NO: 2-25) that, once the target double stranded polynucleotide is assembled, will be identical in sequence to the SOL

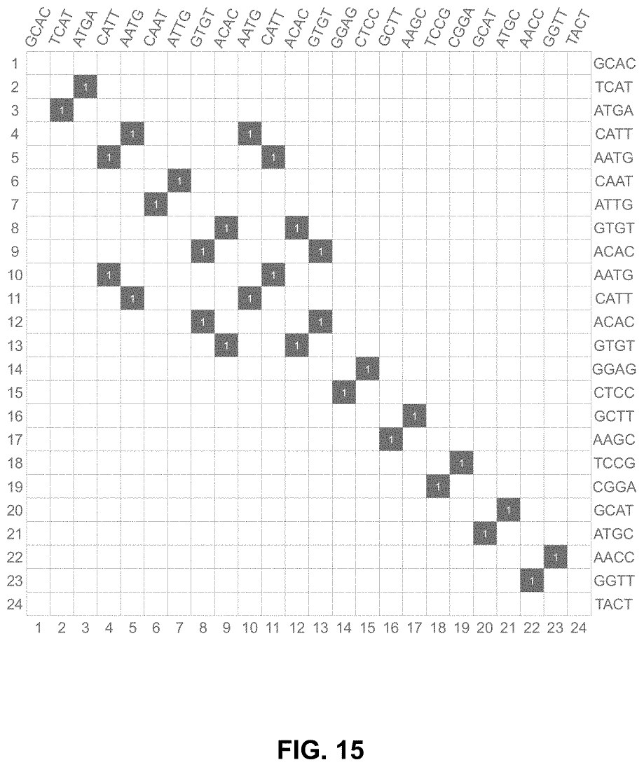

FIG. 15 shows the adjacency matrix described above for this specific partition. Each coloured element represents a pair of overhangs that can be ligated successfully. Not all these overhangs are exposed to each other during the assembly process, rather, it is the assembly tree to determine which overhangs will come into contact. A successful assembly is one when only successive double stranded oligonucleotides ligate properly, which in this matrix representations are those elements in the two sub-diagonals (hence the nonzero elements of the ligation matrix); all the other non-zero elements are to be avoided (M_{4,10}, M_{5,11}, M_{8,12}, . . . in this example). It is possible then to correlate a specific assembly tree to the likelihood of a successful assembly, hence making it possible to find an optimal tree.

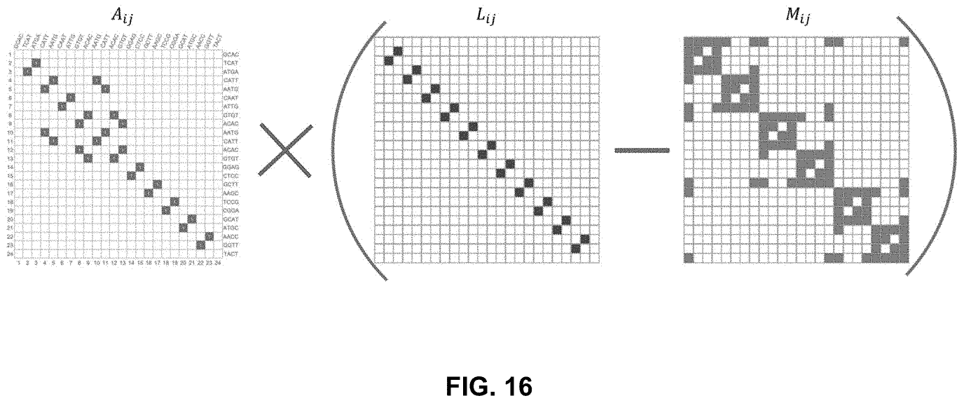

FIG. 16 shows a graphical representation of the scoring obtained with the formula given above, assuming an arbitrary, non-optimal, assembly tree to calculate the misligation matrix.

Determining the Optimal Assembly Tree

Any assembly tree, representing an assembly workflow, can be represented as a “grouping”, i.e., a representation of brackets that details which fragments are assembled first. For instance, if there are 3 fragments a, b and c, the assembly tree representing the ligation of b.c first, followed by the addition of a would be {a,{b,c}}.

In order to determine the optimal assembly tree, the totality of all possible groupings of the fragments can be generated, corresponding to all possible assembly trees. For 12 fragments, there are a total of 58786 possible assembly trees.

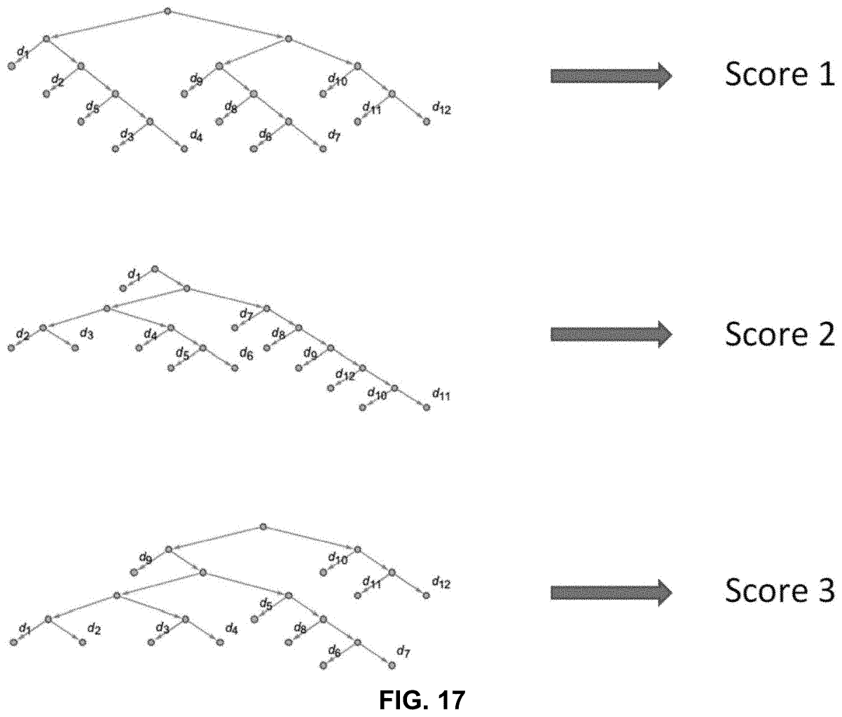

FIG. 17 shows that each of these trees have a corresponding score, representing the likelihood of the success of the assembly process. The algorithm will score each of these groupings according to the metric defined above and determine which assembly tree is the most likely to produce a successful assembly, because it will avoid connecting the nodes that have a high chance of form a misligation. If multiple trees have the same optimal score, the one which requires the smallest number of steps in an automation setting will be selected.

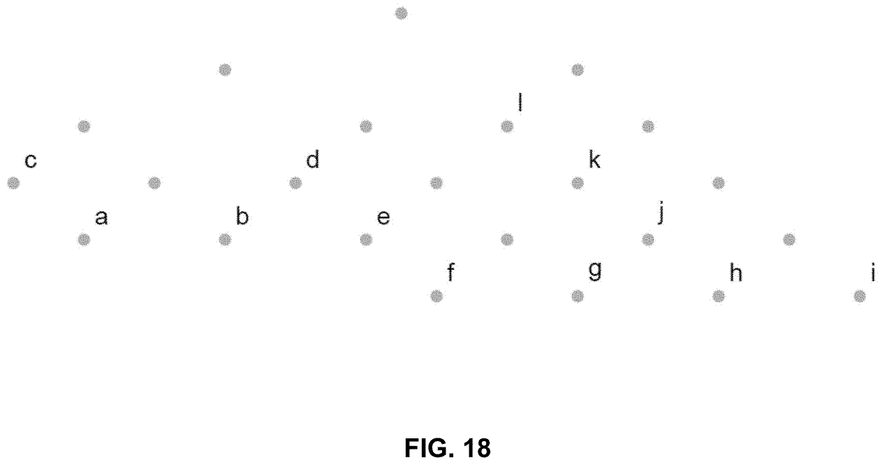

FIG. 18 gives the optimal assembly tree for the specific case of the SOI. For the depicted tree, the corresponding grouping is {{{{a, b}, c},{d, {e, {f, g}}}}, {{{{h, i}, j}, k}, l}}.

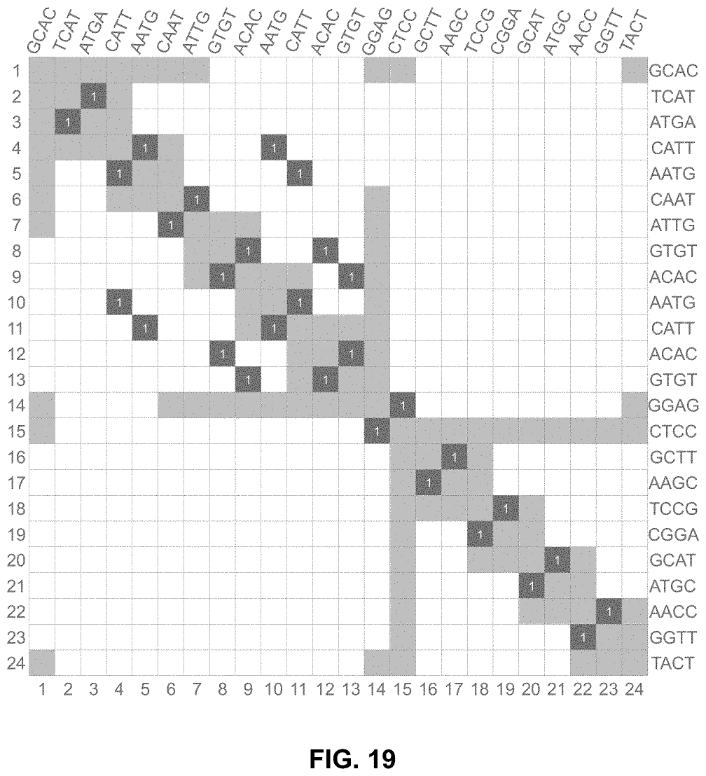

FIG. 19 shows the overhang adjacency matrix for the assembly defined by the tree in FIG. 18. The dark grey areas in the matrix represent the elements of the misligation matrix, hence those overhangs that are in contact at any point during the assembly, and indeed all the misligation events are avoided by this optimal assembly tree.

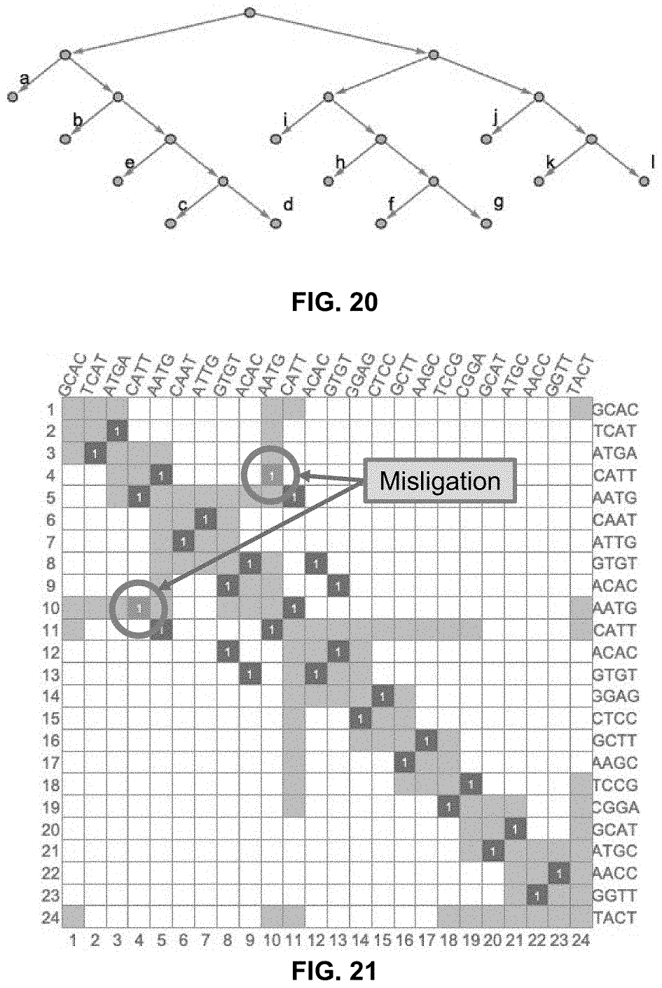

FIG. 20 shows an alternative assembly tree

FIG. 21 gives a corresponding adjacency matrix, but in this case a misligation event is not avoided by this suboptimal tree, as highlighted in the figure.

Provided a well-defined scoring metric, this method guarantees to always find the assembly tree that maximizes this score, since it scans through all possible trees and determines the highest ranking one.

### Example 2. Different Scoring Functions Correspond to Different Optimal Assembly Trees

The choice of the objective function is fundamental in the outcome of the tree optimisation process. In this example we will show how different scoring functions will ultimately correspond to different assembly trees.

Defining Multiple Scores

For this example, we will consider 3 possible scoring. (S1) The first one is obtained by considering perfect matching between the overhang: If two overhangs have perfect Watson-Cricket match on all 4 bases then the specific matrix element of Aᵢⱼ is 1, otherwise the element is zero, then the overall scoring is calculated with the same method as done in Example 1. (S2) The second scoring assigns a value of 3 to any GC pairing between the corresponding overhangs and a value of 1 for any AT pairing. (S3) The third calculates the energy of hybridization of the overhangs and, if the energy is above a specific threshold, it assigns the energy value rescaled into an interval between 0 and 10.

Determining Optimal Assembly Trees

Using these scoring functions, we have calculated the optimal assembly trees for a sequence of 128 bp (SEQ ID NO: 26), which was first partitioned naively into 16 bp fragments (SEQ ID NO: 27-42).

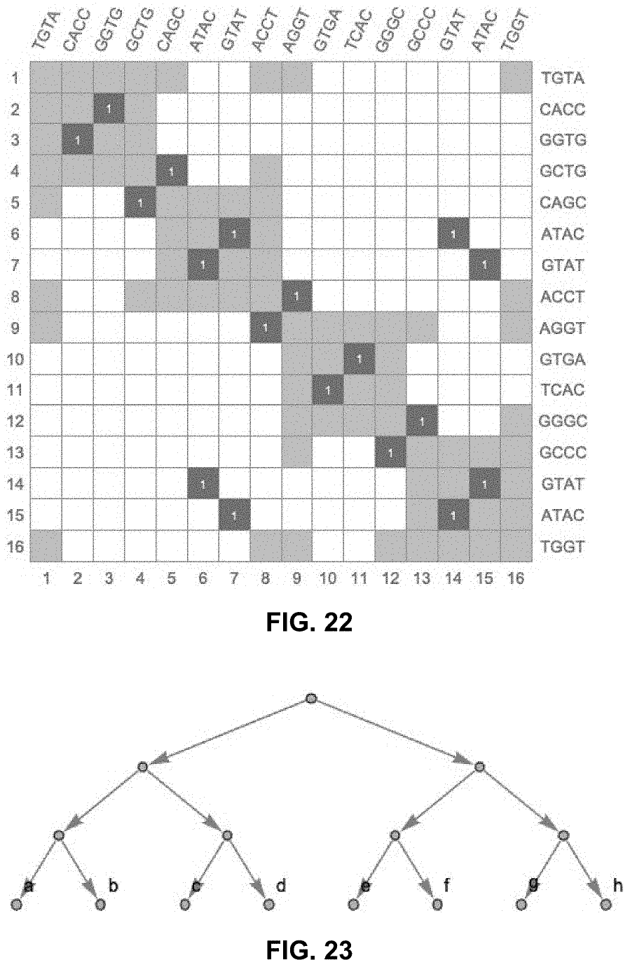

FIG. 22 gives an adjacency matrix with scoring S1.

FIG. 23 shows the assembly tree with the S1 scoring.

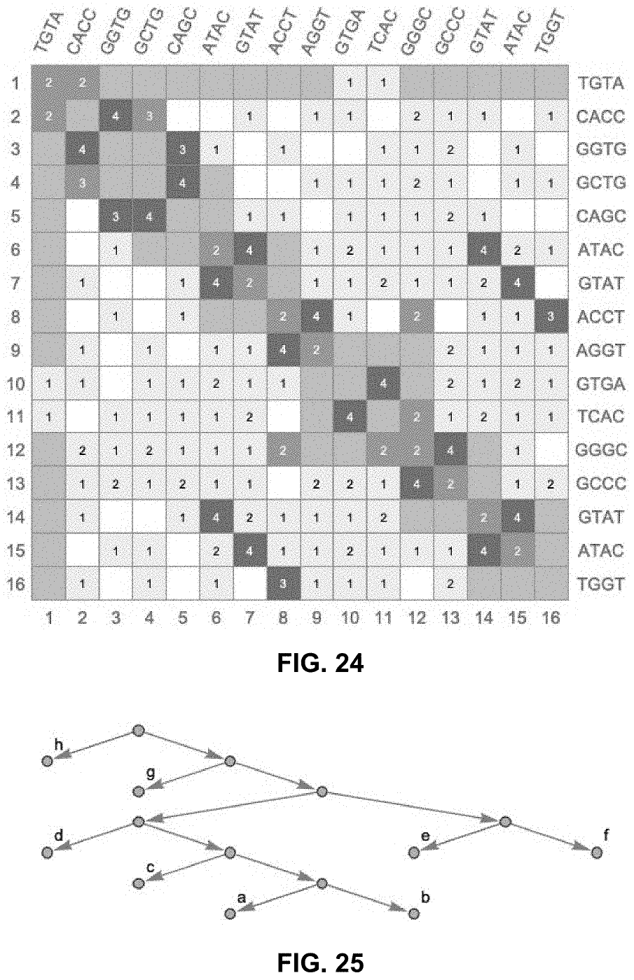

FIG. 24 shows an adjacency matrix with scoring S2.

FIG. 25 shows the assembly tree with S2 scoring.

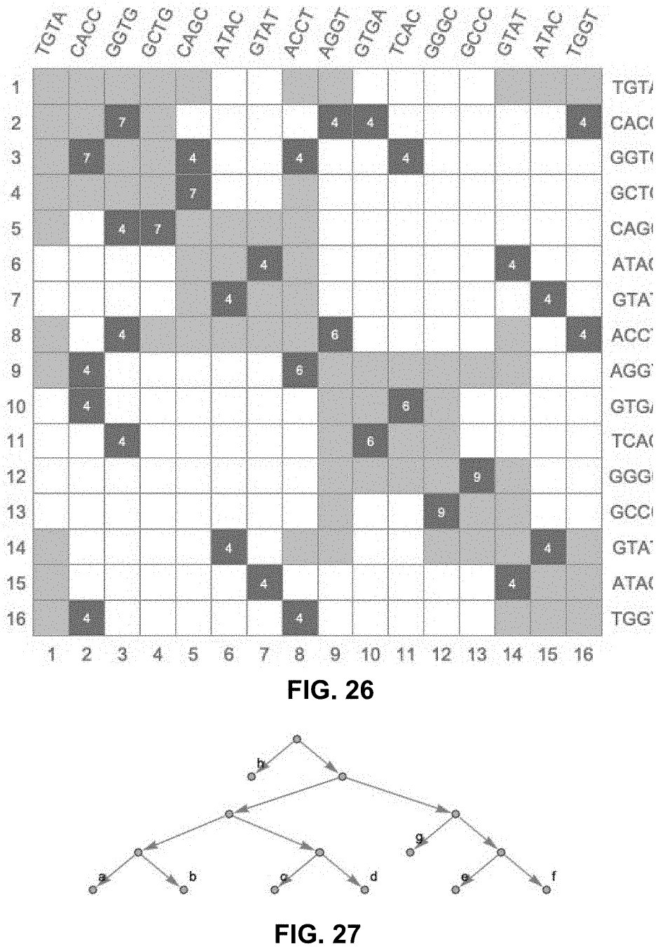

FIG. 26 shows an adjacency matrix with scoring S3.

FIG. 27 gives the assembly tree for the S3 scoring.

FIGS. 22-27 shows the three results by comparing the Adjacency matrices and the assembly trees.

For the S1 scoring a fully symmetric assembly tree seems to be the most likely one, since the only perfectly matching overhang pairs are those that are part of the ligation matrix, except for a pair (elements {6,14}, {7,15}) which anyway are never in contact during the symmetric assembly (as shown by the greyed boxes, corresponding to the elements of the misligation matrix). On the other hand, when considering scoring S2, the number of matching pairs increases dramatically, so does the combination of possibly misligating; for these reasons, the corresponding optimal tree has more asymmetry, and although it minimises the number of potentially misligating combinations it is not able to avoid them completely (dark shaded elements of the adjacency matrices). The S3 scoring is a middle ground solution, since the energy requirement on the overhang optimisation is more difficult to meet for the overhang pair, yet it does not require a perfect match (as it is for 51); for these reasons, the number of non-zero elements of the adjacency matrix is larger, but the optimal assembly tree is still able avoid all problematic combinations.

### Example 3. Assembly of a Target Molecule of 256 bp with an Asymmetric Workflow and Comparison to a Symmetric Workflow by Ligation of 16-Mers

After partitioning the target sequence (SEQ ID NO: 43) and defining the assembly trees as described in Example 1, the oligos are assembled by following both a symmetric assembly and by following the asymmetric tree.

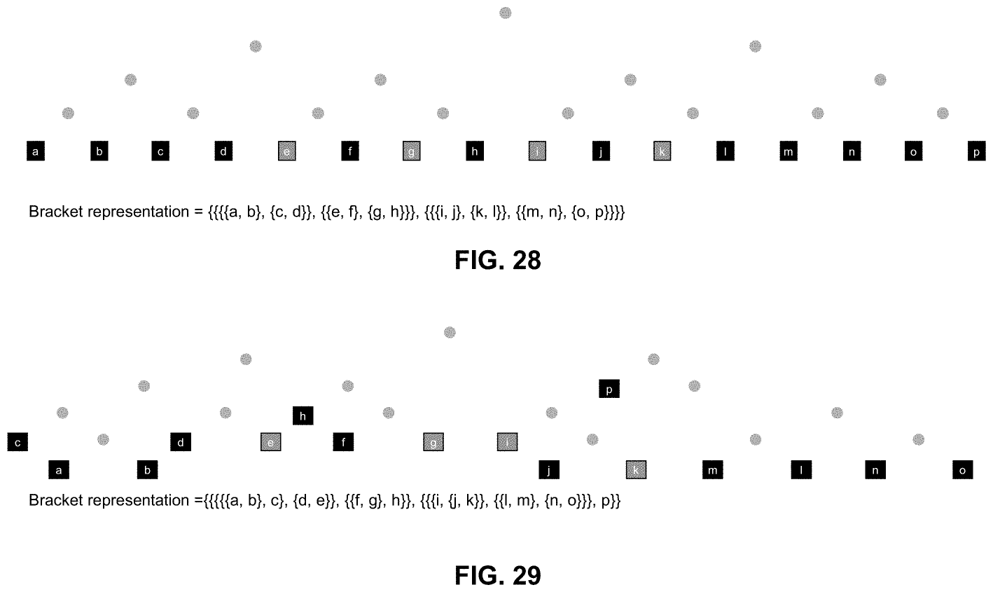

FIG. 28 shows the symmetric assembly.

FIG. 29 show the asymmetric assembly. The asymmetric assembly is optimized by using the algorithmic procedure described in Example 1.

All oligos a-p were procured by Integrated DNA Technologies (IDT) with standard desalting purification and were provided normalized at a concentration of 50 μM on IDTE Buffer (pH 7.5). The oligos used in the assemblies below were single-stranded and pure.

Preparing the Annealing Solutions and Annealing

Some commercial buffers are ready to mix in H₂O such as New England Biolabs' (NEB) T4 Ligase Reaction Buffer, product nr B0202S, and readily contain the ATP necessary for the ligase activity. In a microcentrifuge tube, 216 μl of ddH₂O with NEB T4 ligase buffer were prepared and the solution was mixed well by vortexing. 24 μL of this solution mix were dispensed into to 8 reaction tubes labelled a, b, p. 3 μL of each oligo was transferred to 8 predefined tubes and mixed well by pipetting:
Oligos a− and a+ into tube a
Oligos b− and b+ into tube b
Oligos c− and c+ into tube c
Oligos d− and d+ into tube d
Oligos e− and e+ into tube e
Oligos f− and f+ into tube f
Oligos g− and g+ into tube g
Oligos h− and h+ into tube h
Oligos i− and i+ into tube i
Oligos j− and j+ into tube j
Oligos k− and k+ into tube k
Oligos l− and l+ into tube l
Oligos m− and m+ into tube m
Oligos n− and n+ into tube n
Oligos o− and o+ into tube o
Oligos p− and p+ into tube p

The tubes were sealed and incubated in a thermocycler for 30 sec at 98° C. The temperature was then decreased from 95° C. to 24° C. with a ramp function that diminished the temperature by 1° C. per minute allowing the matching pairs of ss oligos to anneal. Once finished the double stranded oligos were kept at 4° C.

Preparing the Ligation Solution.

The ligation solution was prepared on ice by mixing, in the following order, 32.5 μL of nuclease free ddH20, 7.5 μL of ligase buffer and 5 μL of ATP for a final concentration of 10 mM. The ligation solution was mixed well by vortexing and spun down. 2.5 μL of T4 Ligase (NEB, product nr. M0202) & 2.5 μL of T4 polynucleotide kinase (PNK) (NEB, product nr. M0201B) were added for a total of 10 and 0.25 units per μL of final solution, respectively, and mixed well by gently pipetting. The solution was kept on ice until needed. 10 μL of the ligation solution were transferred to each of 4 tubes (b, d, f, h) containing 5 μL the corresponding ds oligos and mixed by pipetting. Afterwards the tubes were sealed again.

Symmetric Assembly

The oligos are arrayed in rows on a 96-micro-well plate and pairs of oligos or reaction products are transferred in tiers:
i. First assembly tier. Transfer the contents of oligo mixtures a, c, e, g, l, k, m and o into mixture of oligo b, d, f, h, j, l, n and p respectively (plus ligation solution).
ii. After gently mixing by pipetting, these two reactions were incubated for 30 min at 24° C.
iii. Second assembly tier. Transfer the contents of oligo mixtures ab, . . . , mn (tubes b, . . . , n) into mixture of oligo cd, . . . , op (tube d, . . . , p).
iv. After gently mixing by pipetting, these two reactions were incubated for 30 min at 24° C.
v. Third assembly tier. Transfer the contents of oligo mixtures a.d (tube d) into mixture of oligo e.h (tube h) and the contents of oligo mixtures j.l into mixture of oligos m.p (tube p).
vi. After gently mixing by pipetting, these two reactions were incubated for 30 min at 24° C.
vii. The final volume containing the 128 bp product was 80 μL.
viii. A bead purification of 128 bp products was performed by using MagMax bead technology and following manufacturer's instructions with the following modifications to the protocol: binding solution buffer to sample ratio used was 1.66. After beads purification, DNA was eluted in 17 μl of ligation mix (T7 ligase (75 U/μl), T4 PNK (0.25 U/μl), 10 mM ATP, 10× T4 NEB Buffer and H₂O)
i. Fourth assembly tier. Combine the reaction product a.h (tube h) with the mixture i.p (tube p).
ii. Gently mix by pipetting and incubate the reaction for 60 min at 24 C.
iii. A bead purification of final 256 bp products was performed by using MagMax beads following the manufacturer's protocol with the following modifications to the protocol: binding solution buffer to sample ratio used was 1, elution in 17 μl ddH₂O.

Asymmetric Assembly by Step-by-Step Hand-Pipetting

In this case, it suffices to keep track of the order in which the oligos and their reaction products are combined, by proceeding according to the tree in FIG. 10b. The reaction conditions are the same as in the symmetric assembly. Although the asymmetric assembly will follow the steps in a similar manner as with the symmetric assembly, it is important to notice that the order of the transfers is crucial.
iv. First assembly tier. Transfer the contents of oligo mixtures a (tube a) into mixture b (tube b), oligo mixtures j (tube j) into mixture k (tube k), oligo mixtures m (tube m) into mixture l (tube l) and oligo mixtures n (tube n) into mixture o (tube o).
v. After gently mixing by pipetting these two reactions were incubated for 30 min at 24° C.
vi. Second assembly tier. Transfer the contents of oligo mixtures a.b (tube b) into mixture c (tube c), the contents of oligo mixture d (tube d) into mixture e (tube e), oligo mixtures f (tube f) into mixture g (tube g), oligo mixtures i (tube i) into mixture j.k (tube k) and oligo mixtures m.l (tube l) into mixture n.o (tube o).
vii. After gently mixing by pipetting these two reactions were incubated for 30 min at 24° C.
viii. Third assembly tier. Combine the reaction product a.c (tube c) with the mixture d.e (tube e), oligo mixtures f.g (tube g) into mixture h (tube h), oligo mixtures i.k (tube k) into mixture m.o (tube o).
ix. Gently mix by pipetting and incubate the reaction for 30 min at room temperature.
x. Fourth assembly tier. Combine the reaction product a.e (tube e) with the mixture f.h (tube h) and oligo mixtures i.o (tube o) into mixture p (tube p).
xi. The final volume containing the 128 bp product was 70 μL.
xii. Heat inactivate the ligation reaction by incubating for 10 min at 65° C.
xiii. A bead purification of 128 bp products was performed by using MagMax bead technology and following manufacturer's instructions with the following modifications to the protocol: binding solution buffer to sample ratio used was 1.66. After beads purification, DNA was eluted in 17 μl of ligation mix (T7 ligase (75 U/μl), T4 PNK (0.25 U/μl), 10 mM ATP, 10× T4 NEB Buffer and H₂O)
xiv. Fifth assembly tier. Combine the reaction product a.h (tube h) with the mixture i.p (tube p).
xv. Gently mix by pipetting and incubate the reaction for 60 min at 24 C.
xvi. A bead purification of final 256 bp products was performed by using MagMax beads following the manufacturer's protocol with the following modifications to the protocol: binding solution buffer to sample ratio used was 1, elution in 17 μl ddH₂O.

Visualization and Comparison of the Results

Following ligations, final products were subjected to quality control using capillary electrophoresis (Fragment Analyzer). Prior to the loading of the samples on the FA, samples were diluted 2× in 1×TE Buffer. Samples were run on the FA using Small Fragment Kit (Agilent, DNF-476-0500) and using corresponding SS Small Fragment method (Agilent, DNF-476-33) following manufacturer's instructions.

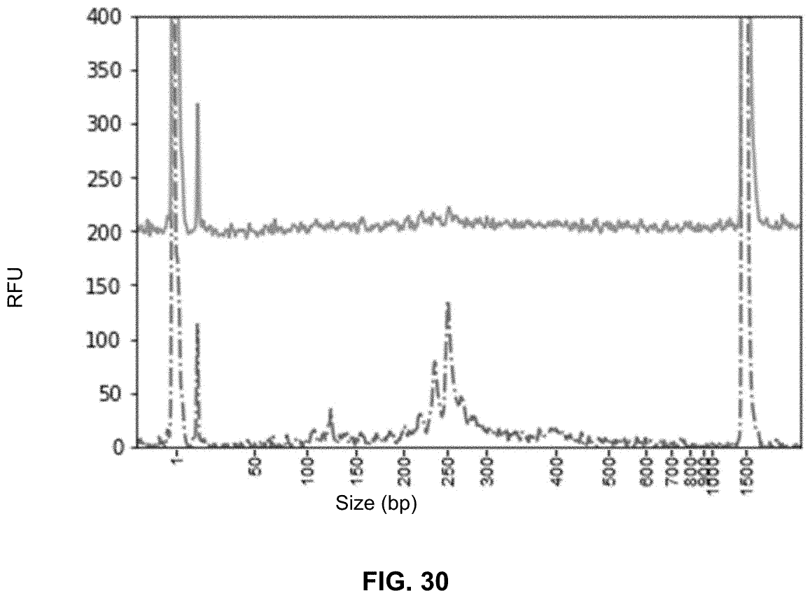

FIG. 30 shows the electropherograms of the two assemblies as obtained after the purification procedure. The symmetric assembly fails, since there is no clear peak at the target length and the target molecule could not be recovered even with purification. In contrast, the asymmetric assembly leads to results where the target molecule can be recovered after purification as seen in a clear peak.

Optionally, the successful construct can be isolated from the gel by using standard kits (e.g. Zymoclean), amplified by PCR (e.g. Sambrook and Russell, 2014; Chapter 8) and sequenced.

### Example 4. Pseudo-Code for a Monte Carlo Algorithm to Find an Optimal Sequence Partition and/or Assembly Graph

This example describes a Metropolis algorithm for identifying an optimal sequence partition and tree search. Metropolis algorithms are described the literature of stochastic search, statistical mechanics, heuristics and many other fields in science, mathematics and computer science. This example presents an adaptation of said algorithm in order to generate an optimal or nearly optimal assembly graph that consists of both, the sequence partition and the assembly graph properly. Similar algorithms for constructing phylogenetic trees in evolution can be found in the literature, which have certain analogies to the construction of graphs in this example. However, the phylogenetic interpretations of the trees, scores, and other properties differ from the ones of this field of application. For example, differences include that the graphs need not to be binary and we may want to allow for nodes that have more than two branches, or that in phylogenetics the tree applies to all nucleotides in the sequences independently whereas in our case the assembly tree applies to the sequence as whole. Also, in phylogenetics every node represents a nucleotide, whereas in our case internal nodes represent partial assemblies. Nevertheless, certain algorithms from phylogenetics, eg. for generating tree variants (Felsenstein 2004; Gascuel 2005; Gascuel and Steel 2007), can be innovatively applied to the field of DNA synthesis.

This example of the Metropolis algorithm reads a string that is represents the target sequence (Target_Seq). Calling the function PARTITION computes an in initial partition of the target sequence into an array of shorter strings that catenated in an orderly manner would recover Target_Seq. For example, PARTITION can return a homogeneous partition into substrings equal length s, or can be heterogeneous lengths making substrings of length in a range (sₘᵢₙ,sₘₐₓ) either randomly or by some criterion. This array is initialized into three variables that are used later through the process in order to iterate the search (Curr_Part), propose a new partition (New_Part), or store the best partition (Best_Part). Similarly, an initial graph is generated by a function MAKETREE, which takes a partition (array of strings) and returns a graph object and, concomitantly, this initial tree is scored by the function SCORETREE which takes a partition and a graph object as arguments and assigns score to the tree, as in examples 1 and 2. As with the output of PARTITION, also three variables for the initial graph and score are initialized in order to iterate and store the best combination of partitions and trees. (The graph object could readily contain the partition and the score but for clarity we explicitly separate the three.)

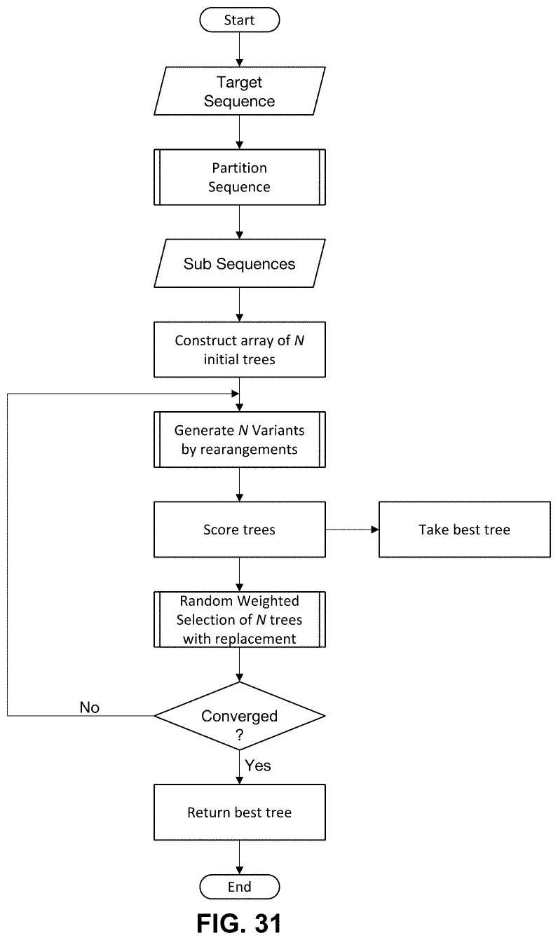

FIG. 31 shows steps of a method.

After the initialization, an iterative loop is entered where a variant of the partition or of the graph is generated, aiming at generating local changes that stochastically seek an optimum of the assembly graph—including the partition. The generation of variants can be done in multiple ways but in this example we assume that we randomly pick a node of the graph, for example with probability inversely proportional to the score of the node and attempt to generate a variant that improves it. We assume two possible types of variants or “flips”. The first (Flip_Type=1) the function REPARTITION modifies the sub-sequences in a node by, eg. moving the 3′ nucleotide of a sequence in the node to the 5′ of its contiguous (rightmost) sub-sequence. For example, if the node consists of two subsequences (N . . . NT,GN . . . N), the algorithm gives (N . . . N,TGN . . . N). The converse can also be done, i.e. moving the 5′ nucleotide of a sequence in the node to the 3′ of its contiguous leftmost sub-sequence in the node. For example, (N . . . NT,G . . . N)->(N . . . NTG,N . . . N). In the second variant generation type (Flip_Type=2) the topology of the graph is changed by the function REBRANCHTREE which changes an immediate vertex in the sub-tree. For example, if the focal node has the sub-tree ((s₁,s₂),(s₃,s₄)) it can rebranch as (s₁,(s₂,(s₃,s₄)), ((s₄,(s₂,s₃,)),s₄), etc. Note that Flip_Type=1 would affect the score of all the sub-branches whereas Flip_Type=2 affects only the immediate nodes. For efficiency, we could assign different frequencies for these types of changes and allow them to be adaptive to the state of the process, but in this example we keep them 50%-50%.

As the sequence or graph variants are generated and the graph is scored, these are respectively assigned to the variables New_Part, New_Tree and New_Scor. These “proposal” state is then compared to the current state by evaluating whether New_Scor improves Curr_Scor (New_Scor>Curr_Scor?). If so, the new state is accepted by assigning it to the current configuration of partition, tree and score. If the new score does not improve we compute RFact, (can be the Boltzmann factor RFact=EXP(−B*(Curr_Scor−New_Scor)) with B>0, or directly the ratio RFact=New_Scor/Curr_Scor) and draw a uniform random number in (0,1). If the number is lower than RFact then the new configuration is accepted by assigning it to the current configuration of partition, tree and score; otherwise, we keep the current configuration and discard the proposal.

In addition, we compare whether the new configuration is better than the best configuration found up this point. If so, the new configuration is assigned to the best configuration of partition, tree and score.

This process of generating and evaluating variants is iterated exhaustively. Because the algorithm never terminates a stop criterion is needed. In this example we add a counter Search_Steps that keeps track how often it has been since the last update of the optimum. If the best configuration has not changed in the last LIMSEARCH iterations (e.g. LIMSEARCH=1000000 times since last update of the best configuration) then the loop breaks and the best configuration is returned. Other criteria can be implemented instead.

```
READ: string Target_Seq
SET: string* Curr_Part=New_Part=Best_Part=PARTITION(Target_Seq)
SET: graph Curr_Tree=New_Tree=Best_Tree=MAKETREE(Curr_Part)
SET: double Curr_Scor=New_Scor=Best_Scor=SCORETREE(Curr_Part, Curr_Tree)
SET: int Search_Steps=0
WHILE Search_Steps <= LIMSEARCH
  SET: int N_Nodes=COUNTNODES(Curr_Tree)
  SET: int Focal_Node=RAND(1,N_Nodes)
  SET: int Flip_Type=RAND(1,2)
```

| | |
|---|---|
| IF Flip_Type==1 | //make a modification in the partition |

```
  SET: New_Part = REPARTITION(Curr_Part,Focal_Node)
  SET: New_Tree=Curr Tree
```

| | |
|---|---|
| ELSE IF Flip_Type==2 | //make a modification in the tree |

```
    SET: New_Tree=REBRANCHTREE(Curr_Tree,Focal_Node)
    SET: New_Part=Curr_Part
    ENDIF
  ENDIF
  SET: New_Scor=SCORETREE(New_Part,New_Tree) //update the scores
  IF New_Scor > Curr_Scor
    SET: Curr_Part=New_Part
    SET: Curr_Tree=New_Tree
    SET: Curr_Scor=New_Scor
  ELSE IF RAND(0,1) < RFact(Curr_Scor,New_Scor) //eg Boltzmann Factor
      SET: Curr_Part=New_Part
      SET: Curr_Tree=New_Tree
      SET: Curr_Scor=New_Scor
    ENDIF
  ENDIF
```

| | |
|---|---|
| IF New_Scor > Best_Scor | //store if score is best so far |

```
    SET: Best_Part=New_Part
    SET: Best_Tree=New_Tree
    SET: Best_Scor=New_Scor
    SET: Search_Steps=0
  ENDIF
  SET: Search_Steps++
ENDWHILE
RETURN (Best_Part,Best_Tree,Best_Scor)
```

### Example 5: Evolutionary Heuristic Search Algorithm to Find an Optimal Tree

In this example the search for an optimal or nearly-optimal assembly graph is done by a heuristic algorithm that generates populations of graphs and applies (a) a selection criterion based on the scores to generate a “breeding population” and (b) a variation-generation process to generate modifications that are close to the breeding population. Variation can be done in a series of ways and in this example we interpret it as single-vertex modification at a time to each tree, in the same or similar manner as discussed in the previous example. In this example we assume the sequence partition is held fixed, but this an extension to this model may be relaxed. Other modifications could include “recombining” trees or other means to re-draw the “breeding” graphs.

In the current example, the sequence input containing the target sequence is partitioned by a process PARTITION (see prev. example) that gives an array of sub-sequences. From this partition, a population of N graphs, or trees, are generated. These can initially be N copies of the same tree, for example, the most symmetric tree.

Then, the process enters the main iterative loop. Within this loop each tree is (i) modified by a tree rearrangement algorithm (eg. REBRANCHTREE in the previous example) to generate a population that comprises a certain diversity of different trees and (ii) scored after the rearrangements in order to have an objective measure to select from. From this set, the tree with the highest score is stored. A selection criterion is applied to generate the next breeding population of trees. The selection criterion can be directly the score or a function that combines and/or transforms said score with other parameters. For example, a breeding population of N trees can be achieved by random selection with replacement from the N existing trees, with probabilities proportional to their scores or selection functions. We can add constraints, as for example minimizing the asymmetry of the trees whilst increasing the score, or any other convenient constrictions. After scoring, we record the best tree that was generated and if it has higher score than the previously recorded putative best tree, then the latter is replaced by the newly found best tree in the breeding population. Once the breeding population has been generated and its maximum has been evaluated and recorded, the procedure is iterated to a further generation of variants from which we select again, and so on. This process effectively improves the mean average score of the population, plus minus fluctuations and converges to a local optimum.

This algorithm, similar to the Metropolis algorithm of the previous example, also does not terminate, so a further halting criterion can be included. That termination criterion can be as simple as a counter, or a convergence measure that monitors the stability of the evolutionary process.

Once the algorithm is stopped, the best tree is returned. This is a non-trivial graph that guides the assembly of the target sequence, as in Example 1.

### Example 6. Assembly Tree Optimisation of a GC-Rich Target Molecule of 1028 bp with an Asymmetric Workflow by Ligation of Oligonucleotides of Different Lengths Using Liquid Handlers

In this example, the advantage of using an asymmetric workflow and optimization algorithms to partition molecules with challenging sequences into building blocks (oligos) that are of unequal length. The sequence of interest is of 1028 bp and is partitioned in two ways: into fixed-length 16mers and with a size adaptive between 15 and 17 nt. The partitions are dubbed c13 and c14, respectively, for reference. We will compare the scores of the balanced (symmetric) assembly tree for the naïve partition to the optimised assembly trees of both the naïve and the adaptive partitions.

Choosing the Objective Function

In order to score an assembly tree, a “Misligation Matrix” (M_{i,j}) is generated. Each element of this matrix is 1 if the corresponding overhang pair is exposed during the ligation process, as a consequence of the chosen assembly tree, but it is not supposed to ligate; 0 otherwise. The ligation matrix (L_{i,j}) assigns a score (between 0 and 4000) directly related to the chance of ligation of the corresponding pair, irrespective of whether they are exposed during the assembly process. High GC content pairs have a larger score. Specifically, if the two overhangs have at least 3 out of 4 matching nucleotides according to Watson-Crick pairing, the matrix element is increased by a factor 10 for every A/T pair and by a factor 1000 for every G/C pair. To calculate the score, the two matrices are multiplied, element-wise, and then the total sum of each element is taken. Overall the score is:

$$S = - ( {\sum }_{i , j} {L}_{i , j} \times {M}_{i , j} ) / {n}^{2}$$

Where n is the total number of overhangs and the minus sign is necessary to make sure that assembly trees with more misligation propensity have lower scores.

Partitioning the Sequence and Selecting the Assembly Tree

By using the score defined in the previous section an optimal partition and assembly tree is computed under three different conditions.

First, a uniform, “naïve” partition is done by segmenting the SOI and its reverse complement into 16mers (with 4 nt offset to allow for 4 nt 5′ overhangs). Those oligos include SEQ ID NO: 76 through SEQ ID NO: 202. Specifically, SEQ ID NO: 76 through SEQ ID NO: 138 constitute the leading (or “top”) strands of ds oligos, while SEQ ID NO: 139 through SEQ ID NO: 202 constitute the lagging strands. The set is dubbed “c13”.

A symmetric tree is used ad hoc as an assembly workflow. The score is parsed on this partition and topology. The naïve partition resulted in 128 oligonucleotide sequences.

Second, the same naïve partition above is used, but an asymmetric tree is computed in order to minimise misassemblies by means of maximising the score.

Third, an adaptive partition is done by locally maximising the score. Oligonucleotide sequences range between 15 and 17 nt and each dimer is constrained to have to 4 nt 5′ overhangs. This partition resulted in 126 oligonucleotide sequences. Those oligos include SEQ ID NO: 203 through SEQ ID NO: 328. Specifically, SEQ ID NO: 203 through SEQ ID NO: 265 make up the leading strands, while SEQ ID NO: 266 through SEQ ID NO: 328 making up the lagging strands. The set is dubbed “c14”.

After a locally-optimal partition has been completed, an optimal tree is computed by further maximising the score.

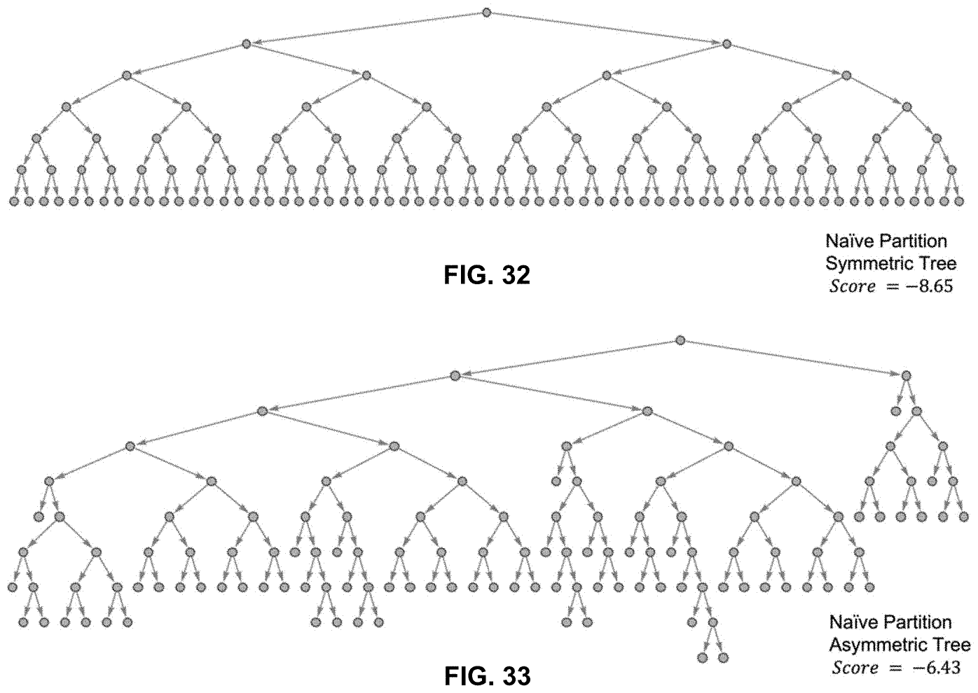

FIG. 32 shows a scored assembly tree for a naïve partition with a symmetric tree,

FIG. 33 shows a scored assembly tree for a naïve partition with the asymmetric tree and

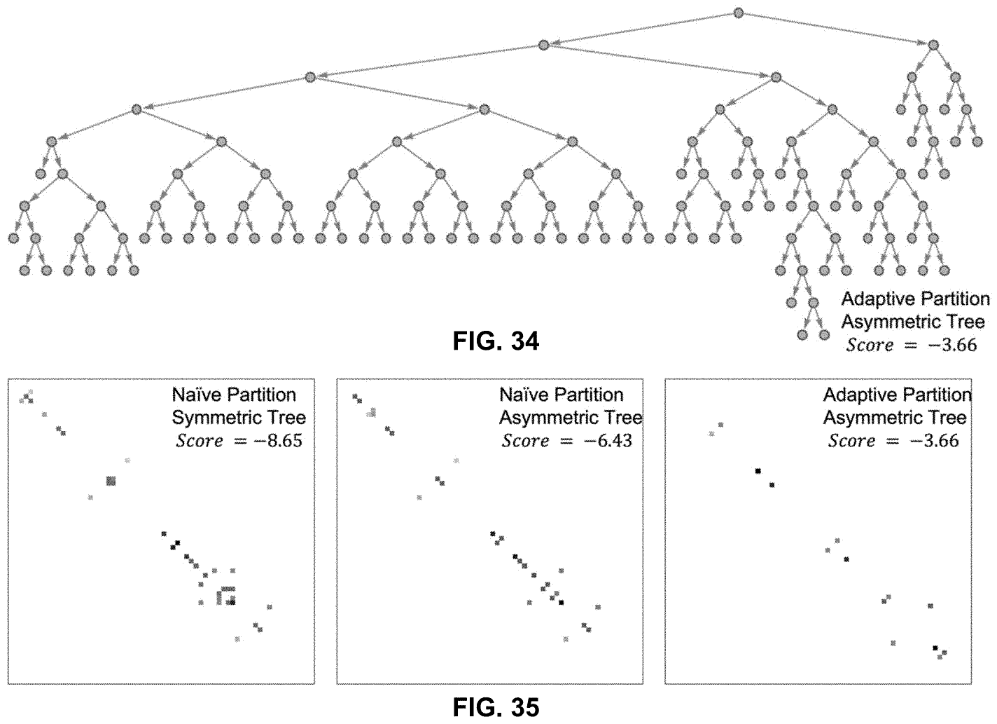

FIG. 34 shows a scored assembly tree for an adaptive partition with a symmetric tree.

FIG. 35 gives the graphs showing, for each case, the product of the ligation and misligation matrix: More darkened elements correspond to lower scores.

As it appears evident from the scoring, the best result is obtained when the adaptive partitioning method meets the asymmetric assembly tree. In this case the score is increased by more than a factor two. The choice of an asymmetric tree implies additional steps are required for the assembly, but it is largely compensated by the extremely increased chances of a successful assembly, even more so for a sequence like the SOI that has a GC content larger than 60%.

Provision of the Oligonucleotides

The synthesis of the oligos was outsourced to an established genomic company. The oligo providers were contacted to ensure that arraying included empty wells as required.

For the oligos from the naïve partition with symmetric tree, 1 plate of 364 micro wells was procured, reflecting the systematic order of the oligos for the symmetric assembly.

The oligos of the naïve partition with an asymmetric tree were procured in 1 plate of 364 micro wells. Even though these are the same oligos as in the symmetric tree, we chose to acquire the oligos readily arrayed and avoid potential mistakes. The arraying was done by re-casting the oligos into a larger symmetric tree and adding zeromers to the rightmost unassigned terminal branches of the tree. The same logic and procedure was followed to procure the oligos of the adaptive partition and asymmetric tree.

Assembly of the Sequences

Once oligonucleotides have been provided, these were annealed as in Examples above to form dimers with 5′ overhangs. The assembly steps were implemented through symmetric movement of an MCA arm on a Tecan Fluent under the same conditions as in Examples above.

Comparison of the results by means of capillary electrophoresis indicated that the naïve assembly with the symmetric tree failed (no detectable peak). The naïve and adaptive partitions with asymmetric trees both resulted in detectable peaks in the electropherogram. The yield and purity proved to be a higher for the assembly based on the adaptive partition with asymmetric tree than on the naïve partition with asymmetric tree.

A PCR amplification of the target product was performed to enrich the sample. After amplification and clean-up with a Zymoclean kit, sequencing was performed on an Oxford Nanopore minION. The analysis of sequencing results indicate that the assembly is correct.

Assembly on Acoustic Dispensers

In a different embodiment, the transfer of oligonucleotides can be achieved by means of acoustic transfer (Echo Liquid Handler 525, Beckman-Coulter). In this embodiment, the arraying with zeromers is unnecessary and it is possible to maximise use of the micro-well plates. Instead of mapping a tree into an array that is to be handled with symmetric movements of a multi-channel pipette, the mapping is performed as “worklist”, i.e. a set of instructions (for example in XML format) that indicate the precise order of transfers to be done. Whilst this is not strictly parallel, the accelerated speed of acoustic dispensing makes it effectively parallel, e.g., as compared to the required incubation time of the reactions.

### Example 7. In this Example Bayesian Learning is Used to Improve Scoring of a Binary Assembly Tree by Using Sequencing Data from Assemblies

Choice of a Surrogate Scores, Partition of the Sequence and Estimation of Assembly Tree

First, for a Sequence of Interest (SOI) a partition and tree i are computed as in Examples above. The score used in this part of the process is understood to be only a surrogate set of parameters \tilde{p} to achieve a good partition. Because the goal of this example is to demonstrate how a Bayesian scheme can be used to improve knowledge about these parameter to improve scoring, estimation and construction of assembly trees, the initial choice should strongly influence the results. (We may as well choose an assembly tree randomly.)

Assembly of the Sequence

As a second stage of the process, once having determined an assembly tree, the assembly is completed in an equivalent manner as in Examples above using liquid handlers and the appropriate enzymatic milieux.

Next Generation Sequencing Data

Third, after having assembled the molecule, sequencing is done with an Oxford Nanopore minION to determine the breadth of the resulting construct after the assembly process. From the sequencing reads the data is parsed to count correct and alternative assemblies as well as unassembled fragments and the frequency f of the target molecule with the SOI is computed. Mathematically, calling f the frequency of the target construct

$$f = \frac{{N}_{SOI}}{{N}_{SOI} + {N}_{m} + {N}_{c 1} + {N}_{c 2}}$$

where Nᵢ is the number of sequence reads of the construct i, SOI is the target construct with the Sequence of Interest, m are the number of misassembled constructs and c1 and c2 are the two constituents of the SOI.

Bayesian Scheme for Computation of Parameter Distribution

After gathering the data, an estimation of the distribution of parameters is done in a Bayesian manner. For instance, the informed distribution P of parameters p given the data f is, according to the Bayesian paradigm

$$P [ p \vert f ] = \frac{ℒ ( f ; p ) P [ p ]}{P [ f ]}$$

where is the likelihood of the data given the parameters, P[p] is the prior distribution of the parameters, and P[f] is the marginal distribution of the data. Despite having chosen a surrogate set of parameters \tilde{p}, at this stage said choice is ignored. Instead, it is assumed that P[p] follows a certain distribution, described below.

Choosing the Score of the Likelihood Function.

In this example we assume that the topology is fixed and concentrate in improving the parameters pᵢⱼ by using the resulting data. In this example the data consists of the sequence reads at the root-node A, which corresponds to the assembled sequence. In the Bayesian scheme

$$ℒ ( f ; p ) := P [ A = f \vert p ] = {\sum }_{B , C} P [ A \vert p , B , C ] P [ B \vert p ] P [ C \vert p ]$$

Because B and C come from separate assemblies, their distributions are independent. These distribution themselves unfold to their respective nodes, and so on.

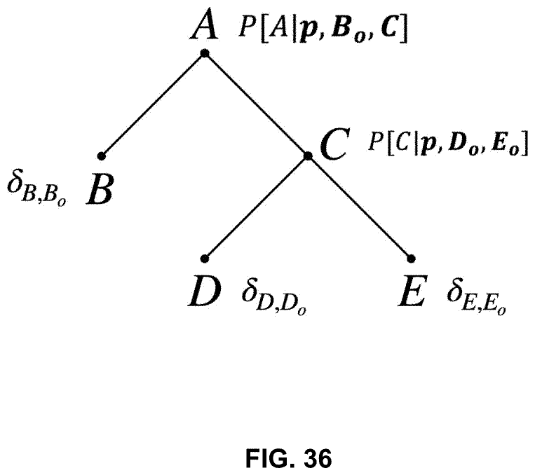

FIG. 36 shows a minimal example of likelihood components in an assembly tree. Capital roman letters are variables (e.g., number of molecules in an NGS read), P denotes likelihood components, p are the parameters of the likelihood (to be estimated), δ_{X,X}_{ₒ} is Kronecker's delta function and the nutted symbols Xₒ are initial number of molecules of building blocks.

To illustrate in a simple tree of FIG. 35,

$$P [ A = f \vert p ] = {\sum }_{B , C} P [ A \vert p , B , C ] P [ B \vert p ] {\sum }_{D , E} P [ C \vert p , D , E ] P [ D \vert p ] P [ E \vert p ] .$$

P[X|p] are the distributions at the nodes which are fixed and therefore a delta function at the starting value P [X|p]=δ_{X,X}_{ₒ}. So, for this simple tree, the likelihood would be

$$P [ A = f \vert p ] = {\sum }_{C} P [ A \vert p , {B}_{o} , C ] P [ B \vert p ] P [ C \vert p , {D}_{o} , {E}_{o} ] .$$

For a more complex tree, as the one in this example, the sums will run over all internal nodes and is implemented in a computer code in an automated manner. Therefore, in this example it is not needed to express the formula analytically.

For the internal probabilities P[T|p, ci, cj] it is considered that the final construct SOI or any target intermediate construct T is constituted by two reagents ci and cj. In addition, it is also considered that misassemblies m are possible. Thus, each node has a certain probability p having n_{T} correctly assembled molecules, nₘ misassemblies and n_{ci} and n_{cj} unassembled reagents ci and cj. The probability of any given state is a multinomial distribution

P[T|p,ci,cj]:=p(n_{T},nₘ,n_{ci},n_{cj})=Cpᵢⱼⁿ^{_{T}}pⱼᵢⁿ^{ₘ}pₒⁿ^{_{ci}}⁺ⁿ^{_{cj}}

where only misassemblies of the types ji are considered in this example. Other models may include self-assemblies ii, jj, and higher order misassemblies iji, ijj, jii, iii, jjj, etc. In this equation C is a normalization constant.

Choosing the Prior

If the probabilities pᵢⱼ are given, they can be used to compute a nearly-optimal tree, in a manner equivalent as above and in Examples above. But despite having used surrogate parameters \tilde{p} to generate the assembly tree, in this Bayesian context the probabilities pᵢⱼ are assumed to be unknown and to follow a prior distribution. In this a non-informative prior is used, i.e., a uniform distribution pᵢⱼ˜U[0,1]. In other embodiment other choices can be used; for instance because in this example the assembly is guided by overlapping overhangs, these probabilities are determined by (a) physical chemical parameters such as molecule length, temperature, ionic concentration, etc. and (b) by the matching accuracy of the overhangs of the assembly. For clarity, different pᵢⱼ can follow destring distributions. It may be assumed that pᵢⱼ is concentrated close to 1 if the overhangs are perfect Watson-Crick pairs, and that this concentration decreases linearly (or otherwise) with the number of mismatches.

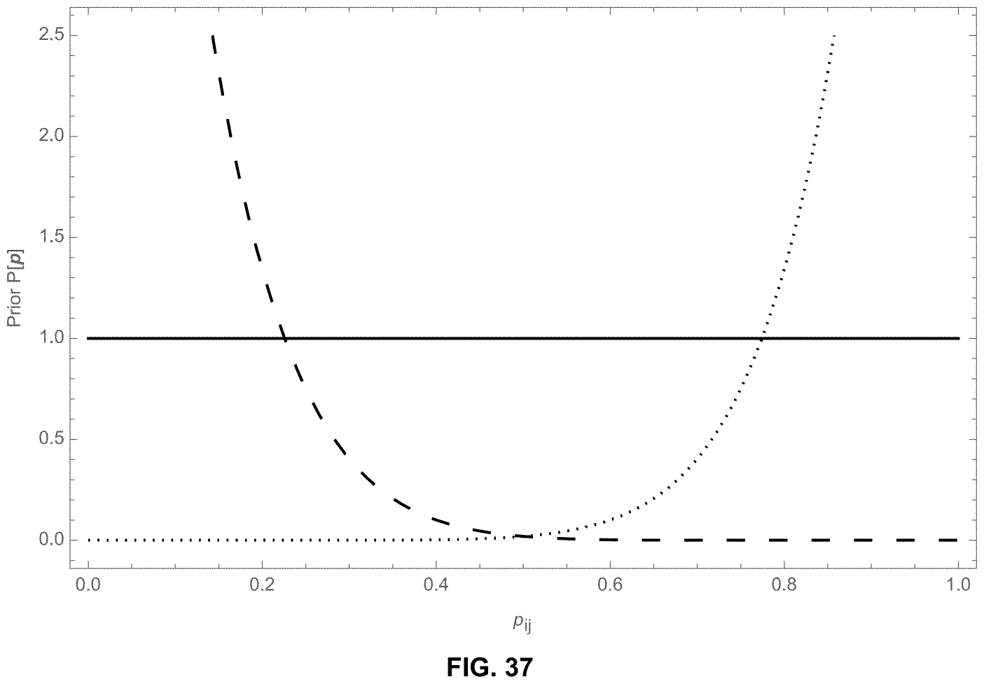

FIG. 37 shows three alternative examples of priors following a Beta distributions. In those possible prior distributions: Solid line: Non-informative, uniform prior (equivalent to Beta[1,1]. Dotted line: Beta distribution favoring perfectly matching overhangs (Beta[1,10]). Dashed line: Beta distribution favoring non-matching overhangs overhangs (Beta[10,1]).

Bayesian Estimation

In this case, the estimation of the posterior parameter distribution is done computationally by implementing a code that combines the formulas above and performs sums numerically. The marginal probability of the data P [f] is achieved by integrating over all parameter values

P[f]=∫ [symbol] (f;p)P[p] [symbol] p

Many methods for numerical sums and integration are available in the literature and in mathematical and statistical software such as Mathematica, Matlab, Maple, Octave, R, amongst others.

The output of the computation is a function or data structure that depends on the input f and may be an evaluable object in a software using certain interpolation methods. In an embodiment, approximations can be used so that each parameter is assumed to follow a normal distribution with certain mean and variance, and only the latter may be used as output.

Iterative Schemes

In a further embodiment of the method, an iterative scheme can be implemented in order to improve the estimated distribution of parameters with cumulative data in the following manner
1. Set initial surrogate distribution Fₒ[p] and set Pₒ[p]=Fₒ[p]
2. Set priors as P[p]=ΠP[pᵢⱼ], where P[pᵢⱼ]=Beta[(aᵢⱼ, bᵢⱼ)] for suitable real, positive numbers aᵢⱼ, bᵢⱼ and i,j run over all possible pairs (including mismatches and self− matches)
3. Draw or select a surrogate choice of parameters \tilde{p} from Pₒ[p]
4. Input an SOI, estimate a tree i using surrogate parameters \tilde{p}
5. Perform the assembly of the SOI in liquid handlers
6. Sequence the end-result to obtain a dataset [symbol]
7. From the sequencing data [symbol] Compute the frequency of the correct data f
8. Compute the likelihood function [symbol] as explained above in this example (a function of the tree topology)
9. Compute the prior P[p]
10. Compute the marginal data distribution P[p]
11. Construct the posterior P[p|f] by integration
12. Output and store the data as an evaluatable function
13. Set the newly computed distribution as new surrogate and new prior, P[p]=Pₒ[p]=P[p|f]
14. Repeat steps 4-14 for new SOIs.

In this manner, every time a new molecules is assembled it generates further information to improve the scoring, thereby converging Xₒ to an accurate predictor. The method can be extended to construct many molecules in parallel and generate a larger dataset from which to improve the parameter estimation.

In another embodiment, a full search may be performed in the parameter space in order to identify the best tree in the product space of trees x parameters, instead of using a surrogate parameters to estimate an optimal tree.

In a further embodiment, a hyper-distribution of trees may be considered to further improve the co-estimation of a tree and parameters based in observed data.

Sequences

>Seq_01 (SEQ ID NO: 1)
AGCCCTCCAGGACAGGCTGCATCAGAAGAGGCCATCAAGCAGGTCTGTTCCAAGGG
CCTTTGCGTCAGGTGGGCTCAGGATTCCAGGGTGGCTGGACCCCAGGCCCCAGCTCT
GCAGCAGGGAGGACGTGGCTGGGCTCGTGAAGCATGTGGGGGTGAGCCCAGGGGCC
CCAAGGCAGGGCACCTGGCCTTCAGCCTGCCTCAGCCCTGCCTGTCTCCCAGATCAC
TGTCCTTCTGCCATGGCCCTGTGGATGCGCCTCCTGCCCCTGCTGGCGCTGCTGGCCC
TCTGGGGACCTGACCCAGCCGCAGCCTTTGTGAACCAACACCTGTGCGGCTCACACC
TGGTGGAAGCTCTCTACCTAGTGTGCGGGGAACGAGGCTTCTTCTACACACCCAAGA
CCCGCCGGGAGGCAGAGGACCTGCAGGGTGAGCCAACTGCCCATTGCTGCCCCTGG
CCGCCCCCAGCCACCCCCTGCTCCTGGCGCTCCCACCCAGCATGGGCAGAAGGGGG
CAGGAGGCTGCCACCCAGCAGGGGGTCAGGTGCACTTTTTTAAAAAGAAGTTCTCTT
GGTCACGTCCTAAAAGTGACCAGCTCCCTGTGGCCCAGTCAGAATCTCAGCCTGAGG
ACGGTGTTGGCTTCGGCAGCCCCGAGATACATCAGAGGGTGGGCACGCTCCTCCCTC
CACTCGCCCCTCAAACAAATGCCCCGCAGCCCATTTCTCCACCCTCATTTGATGACC
GCAGATTCAAGTGTTTTGTTAAGTAAAGTCCTGGGTGACCTGGGGTCACAGGGTGCC
CCACGCTGCCTGCCTCTGGGCGAACACCCCATCACGCCCGGAGGAGGGCGTGGCTG
CCTGCCTGAGTGGGCCAGACCCCTGTCGCCAGGCCTCACGGCAGCTCCATAGTCAGG
AGATGGGGAAGATGCTGGGGACAGGCCCTGGGGAGAAGTACTGGGATCACCTGTTC
AGGCTCCCACTGTGACGCTGCCCCGGGGCGGGGGAAGGAGGTGGGACATGTGGGCG
TTGGGGCCTGTAGGTCCACACCCAGTGTGGGTGACCCTCCCTCTAACCTGGGTCCAG
CCCGGCTGGAGATGGGTGGGAGTGCGACCTAGGGCTGGCGGGCAGGCGGGCACTGT
GTCTCCCTGACTGTGTCCTCCTGTGTCCCTCTGCCTCGCCGCTGTTCCGGAACCTGCT
CTGCGCGGCACGTCCTGGCAGTGGGGCAGGTGGAGCTGGGCGGGGGCCCTGGTGCA
GGCAGCCTGCAGCCCTTGGCCCTGGAGGGGTCCCTGCAGAAGCGTGGCATTGTGGA
ACAATGCTGTACCAGCATCTGCTCCCTCTACCAGCTGGAGAACTACTGCAACTAGAC
GCAGCCCGCAGGCAGCCCCACACCCGCCGCCTCCTGCACCGAGAGAGATGGAATAA
AGCCCTTGAACCAGC
>Oligo a+ (SEQ ID NO: 2)
GCACCAAACGAAAATACTAAGGGCCAACGATACCGGGTGTACGGACACAG
>Oligo a− (SEQ ID NO: 3)
TCATCTGTGTCCGTACACCCGGTATCGTTGGCCCTTAGTATTTTCGTTTG
>Oligo b+ (SEQ ID NO: 4)
ATGATTACGTCTAAATCTCATGCCCAGTAGAAACAGGCGCGGCCAGAAAG
>Oligo b− (SEQ ID NO: 5)
CAATCTTTCTGGCCGCGCCTGTTTCTACTGGGCATGAGATTTAGACGTAA
>Oligo c+ (SEQ ID NO: 6)
ATTGAGGCGATCGCCCACCCGAATGGAGTCACAAGCCACAACCCGTATTG
>Oligo c− (SEQ ID NO: 7)
CAATCAATACGGGTTGTGGCTTGTGACTCCATTCGGGTGGGCGATCGCCT
>Oligo d+ (SEQ ID NO: 8)
ATTGTCTATTGCAATTGATTGCCGCATCATCTAGTGATGAGGACACCTGA
>Oligo d− (SEQ ID NO: 9)
GTGTTCAGGTGTCCTCATCACTAGATGATGCGGCAATCAATTGCAATAGA
>Oligo e+ (SEQ ID NO: 10)
ACACCTTACTCCCTAATTCGTCACCACATTGTTGTGAGCCATAGGGGGAC
>Oligo e− (SEQ ID NO: 11)
GGATGTCCCCCTATGGCTCACAACAATGTGGTGACGAATTAGGGAGTAAG
>Oligo f+ (SEQ ID NO: 12)
ATCCTGTTTAAGCTAGTTAGTTAGCACGTGAAGACGTGGGTTCGGGTTAG
>Oligo f− (SEQ ID NO: 13)
ACGGCTAACCCGAACCCACGTCTTCACGTGCTAACTAACTAGCTTAAACA
>Oligo g+ (SEQ ID NO: 14)
CCGTCCAACCATCTGAAGGGGGAATTGTAAACGTCGGTAGGGCCTGGCAC
>Oligo g− (SEQ ID NO: 15)
GGAGGTGCCAGGCCCTACCGACGTTTACAATTCCCCCTTCAGATGGTTGG
>Oligo h+ (SEQ ID NO: 16)
CTCCTGGCGCTCTCGGTTATCGGTGCTGCGCAAACCCGGAGCAGAGGCGT
>Oligo h− (SEQ ID NO: 17)
GCTTACGCCTCTGCTCCGGGTTTGCGCAGCACCGATAACCGAGAGCGCCA
>Oligo i+ (SEQ ID NO: 18)
AAGCGTGGTCCGACGTGTGAGCGTGCCCGTTAGCGAGTGGGGTCAGGGTG
>Oligo i− (SEQ ID NO: 19)
TCCGCACCCTGACCCCACTCGCTAACGGGCACGCTCACACGTCGGACCAC
>Oligo j+ (SEQ ID NO: 20)
CGGAGTGAACACCACCCCCATTGGTCTAATGCCCTATCAAAGAGAGCCCA
>Oligo j− (SEQ ID NO: 21)
GCATTGGGCTCTCTTTGATAGGGCATTAGACCAATGGGGGTGGTGTTCAC
>Oligo k+ (SEQ ID NO: 22)
ATGCCCTTCGATACCGAATATCCATCTCGGTCGAACCCATATTGCGATAA
>Oligo k− (SEQ ID NO: 23)
AACCTTATCGCAATATGGGTTCGACCGAGATGGATATTCGGTATCGAAGG
>Oligo l+ (SEQ ID NO: 24)
GGTTCTCTGGCGGCAAGACCCTCAGTCGATAACTCCCGGCTAGGGGTATG
>Oligo l− (SEQ ID NO: 25)
TACTCATACCCCTAGCCGGGAGTTATCGACTGAGGGTCTTGCCGCCAGAG
>Seq_02 (SEQ ID NO: 26)
TGTAGTCATGCCCACGGGTGCAAAGCACGAAACAGCAGCGGAGGAGAAGTATCGTA
CGGGGGTCAGGTGACTATAGGTTATCACTGGTAGGGTACCGCCCATAGTCCCCATGA
TACCCGATTCGGTCT
>Oligo a+ (SEQ ID NO: 27)
TGTAGTCATGCCCACG
>Oligo a− (SEQ ID NO: 28)
CACCCGTGGGCATGAC
>Oligo b+ (SEQ ID NO: 29)
GGTGCAAAGCACGAAA
>Oligo b− (SEQ ID NO: 30)
GCTGTTTCGTGCTTTG
>Oligo c+ (SEQ ID NO: 31)
CAGCAGCGGAGGAGAA
>Oligo c− (SEQ ID NO: 32)
ATACTTCTCCTCCGCT
>Oligo d+ (SEQ ID NO: 33)
GTATCGTACGGGGGTC
>Oligo d− (SEQ ID NO: 34)
ACCTGACCCCCGTACG
>Oligo e+ (SEQ ID NO: 35)
AGGTGACTATAGGTTA
>Oligo e− (SEQ ID NO: 36)
GTGATAACCTATAGTC
>Oligo f+ (SEQ ID NO: 37)
TCACTGGTAGGGTACC
>Oligo f− (SEQ ID NO: 38)
GGGCGGTACCCTACCA
>Oligo g+ (SEQ ID NO: 39)
GCCCATAGTCCCCATG
>Oligo g− (SEQ ID NO: 40)
GTATCATGGGGACTAT
>Oligo h+ (SEQ ID NO: 41)
ATACCCGATTCGGTCT
>Oligo h− (SEQ ID NO: 42)
TGGTAGACCGAATCGG
>Seq_03 (SEQ ID NO: 43)
GCACCAAACGAAAATACTATGGGCCAACGATACCGAGTGTACGGACACAGACGATT
ACGTCTGAATCTCATGCCCAGTAGTCGGAGGCGCGGCCAGAGATATTGAGGCGATC
AGCCACCCGAATGGAGAACAAAGCCACAACCCGTATTGCGTCTCTATTTGTTTTGAT
TGCCGCATCATCTAGTGATGAGGGCACCTGAACACCTTAATACCTAATTCGTCACCA
CATTGTTGTGAGCCATAGGGGGACATCCTGTTTA
>Oligo a+ (SEQ ID NO: 44)
GCACCAAACGAAAATA
>Oligo a− (SEQ ID NO: 45)
ATAGTATTTTCGTTTG
>Oligo b+ (SEQ ID NO: 46)
CTATGGGCCAACGATA
>Oligo b− (SEQ ID NO: 47)
TCGGTATCGTTGGCCC
>Oligo c+ (SEQ ID NO: 48)
CCGAGTGTACGGACAC
>Oligo c− (SEQ ID NO: 49)
GTCTGTGTCCGTACAC
>Oligo d+ (SEQ ID NO: 50)
AGACGATTACGTCTGA
>Oligo d− (SEQ ID NO: 51)
AGATTCAGACGTAATC
>Oligo e+ (SEQ ID NO: 52)
ATCTCATGCCCAGTAG
>Oligo e− (SEQ ID NO: 53)
CCGACTACTGGGCATG
>Oligo f+ (SEQ ID NO: 54)
TCGGAGGCGCGGCCAG
>Oligo f− (SEQ ID NO: 55)
ATCTCTGGCCGCGCCT
>Oligo g+ (SEQ ID NO: 56)
AGATATTGAGGCGATC
>Oligo g− (SEQ ID NO: 57)
GGCTGATCGCCTCAAT
>Oligo h+ (SEQ ID NO: 58)
AGCCACCCGAATGGAG
>Oligo h− (SEQ ID NO: 59)
TGTTCTCCATTCGGGT
>Oligo i+ (SEQ ID NO: 60)
AACAAAGCCACAACCC
>Oligo i− (SEQ ID NO: 61)
ATACGGGTTGTGGCTT
>Oligo j+ (SEQ ID NO: 62)
GTATTGCGTCTCTATT
>Oligo j− (SEQ ID NO: 63)
AACAAATAGAGACGCA
>Oligo k+ (SEQ ID NO: 64)
TGTTTTGATTGCCGCA
>Oligo k− (SEQ ID NO: 65)
ATGATGCGGCAATCAA
>Oligo l+ (SEQ ID NO: 66)
TCATCTAGTGATGAGG
>Oligo l− (SEQ ID NO: 67)
GTGCCCTCATCACTAG
>Oligo m+ (SEQ ID NO: 68)
GCACCTGAACACCTTA
>Oligo m− (SEQ ID NO: 69)
GTATTAAGGTGTTCAG
>Oligo n+ (SEQ ID NO: 70)
ATACCTAATTCGTCAC
>Oligo n− (SEQ ID NO: 71)
TGTGGTGACGAATTAG
>Oligo o+ (SEQ ID NO: 72)
CACATTGTTGTGAGCC
>Oligo o− (SEQ ID NO: 73)
CTATGGCTCACAACAA
>Oligo p+ (SEQ ID NO: 74)
ATAGGGGGACATCCTG
>c13leading (SEQ ID NO: 75)
CACTTAGTGCTCGTCC
>c13leading (SEQ ID NO: 76)
CGTTGATCGCTGGTGC
>c13leading (SEQ ID NO: 77)
ATCAGGGCCTTGAGCT
>c13leading (SEQ ID NO: 78)
GGCCTGTTGGTGGGCC
>c13leading (SEQ ID NO: 79)
TGCTAACGCCGCCCTC
>c13leading (SEQ ID NO: 80)
CTGTGGACGAAGTGGA
>c13leading (SEQ ID NO: 81)
ATGAGAGACGTGCGCC
>c13leading (SEQ ID NO: 82)
ATGTTATCTCCGCCAG
>c13leading (SEQ ID NO: 83)
CCGTCCATGCGTGACC
>c13leading (SEQ ID NO: 84)
GGGTCGGGTACGACCG
>c13leading (SEQ ID NO: 85)
GGCCGGAACGGACTCA
>c13leading (SEQ ID NO: 86)
CACCCATGGTGAGCAA
>c13leading (SEQ ID NO: 87)
TACGTATCCACCGGGA
>c13leading (SEQ ID NO: 88)
GCTTAGTTTGGCCCCC
>c13leading (SEQ ID NO: 89)
TTGGGCCGACCCGGGG
>c13leading (SEQ ID NO: 90)
AGTCCGAACGCTCGGG
>c13leading (SEQ ID NO: 91)
ACTACTACTTCACGAG
>c13leading (SEQ ID NO: 92)
CAAGCATCTCCGGCGC
>c13leading (SEQ ID NO: 93)
CGCTGCCCGCGATCTG
>c13leading (SEQ ID NO: 94)
GCACGCCCCTTCCAGA
>c13leading (SEQ ID NO: 95)
CTCCTGGGCAGCGACA
>c13leading (SEQ ID NO: 96)
GCGCCGAGAGGGCGGT
>c13leading (SEQ ID NO: 97)
CGCCCAGGGGAACCGC
>c13leading (SEQ ID NO: 98)
CCTACAGCCCCCGAGA
>c13leading (SEQ ID NO: 99)
TCGTTTCTCCCCCCGC
>c13leading (SEQ ID NO: 100)
TTCCACCCCTGGGCGA
>c13leading (SEQ ID NO: 101)
GGTGCATACCTTTCAA
>c13leading (SEQ ID NO: 102)
CCCGTCAAATGCCACT
>c13leading (SEQ ID NO: 103)
TAGAACGTCCTGCTCG
>c13leading (SEQ ID NO: 104)
TCCGTAGAAAACAGGC
>c13leading (SEQ ID NO: 105)
AGACGCCGGACGCCCA
>c13leading (SEQ ID NO: 106)
TATGGGAGGCGCTCGT
>c13leading (SEQ ID NO: 107)
TGGCCCGCTGGGCGAC
>c13leading (SEQ ID NO: 108)
TGGGCGTGACTGGCGA
>c13leading (SEQ ID NO: 109)
CCGCCAGGTATCCCCG
>c13leading (SEQ ID NO: 110)
GGGGGAGCGCGCCACG
>c13leading (SEQ ID NO: 111)
CTGCAGGCGGACGCGA
>c13leading (SEQ ID NO: 112)
GGGAGATGCAGGGACG
>c13leading (SEQ ID NO: 113)
GGGTGGTCAGCTTGAC
>c13leading (SEQ ID NO: 114)
CCGGGCCTGGGAGGCT
>c13leading (SEQ ID NO: 115)
CGTCCGGCCCCTCGCG
>c13leading (SEQ ID NO: 116)
GCTAGACTGGGATAGC
>c13leading (SEQ ID NO: 117)
GGTATACCAGCGGGAG
>c13leading (SEQ ID NO: 118)
GAGTCTCTCCGGCGTC
>c13leading (SEQ ID NO: 119)
CGGCCCTGCAGGCAAA
>c13leading (SEQ ID NO: 120)
GGCGTGAGCGTTTCCC
>c13leading (SEQ ID NO: 121)
GCTGTCCGGGCGGTAG
>c13leading (SEQ ID NO: 122)
CGGCTGGGGCCATGTC
>c13leading (SEQ ID NO: 123)
CGACGGCGCGCGATGT
>c13leading (SEQ ID NO: 124)
AGTCGGGGCCCATTCC
>c13leading (SEQ ID NO: 125)
ACGGTGCAACGTCCCC
>c13leading (SEQ ID NO: 126)
CTACAGTAGGTCAATC
>c13leading (SEQ ID NO: 127)
GGCAGGCACAGGCGCG
>c13leading (SEQ ID NO: 128)
CGCGCCCGCCGCGCTA
>c13leading (SEQ ID NO: 129)
GTTCCGTGGCCGTCGT
>c13leading (SEQ ID NO: 130)
ACAGAGCAGAGGAATC
>c13leading (SEQ ID NO: 131)
CGAAGTCGGGTTGCCC
>c13leading (SEQ ID NO: 132)
GGGCTCCTGTCCGGCC
>c13leading (SEQ ID NO: 133)
ACCGCCCCGAGCTCCC
>c13leading (SEQ ID NO: 134)
TACGGGACAGCCCGGG
>c13leading (SEQ ID NO: 135)
AAAGCTGGGAGGGACA
>c13leading (SEQ ID NO: 136)
GGGTAGTCGGCCCCCC
>c13leading (SEQ ID NO: 137)
CGCTCTGTCGTTCCGC
>c13leading (SEQ ID NO: 138)
ATCCGCCTTGCGGGCC
>c13lagging (SEQ ID NO: 139)
AACGGGACGAGCACTA
>c13lagging (SEQ ID NO: 140)
TGATGCACCAGCGATC
>c13lagging (SEQ ID NO: 141)
GGCCAGCTCAAGGCCC
>c13lagging (SEQ ID NO: 142)
AGCAGGCCCACCAACA
>c13lagging (SEQ ID NO: 143)
ACAGGAGGGCGGCGTT
>c13lagging (SEQ ID NO: 144)
TCATTCCACTTCGTCC
>c13lagging (SEQ ID NO: 145)
ACATGGCGCACGTCTC
>c13lagging (SEQ ID NO: 146)
ACGGCTGGCGGAGATA
>c13lagging (SEQ ID NO: 147)
ACCCGGTCACGCATGG
>c13lagging (SEQ ID NO: 148)
GGCCCGGTCGTACCCG
>c13lagging (SEQ ID NO: 149)
GGTGTGAGTCCGTTCC
>c13lagging (SEQ ID NO: 150)
CGTATTGCTCACCATG
>c13lagging (SEQ ID NO: 151)
AAGCTCCCGGTGGATA
>c13lagging (SEQ ID NO: 152)
CCAAGGGGGCCAAACT
>c13lagging (SEQ ID NO: 153)
GACTCCCCGGGTCGGC
>c13lagging (SEQ ID NO: 154)
TAGTCCCGAGCGTTCG
>c13lagging (SEQ ID NO: 155)
CTTGCTCGTGAAGTAG
>c13lagging (SEQ ID NO: 156)
AGCGGCGCCGGAGATG
>c13lagging (SEQ ID NO: 157)
GTGCCAGATCGCGGGC
>c13lagging (SEQ ID NO: 158)
GGAGTCTGGAAGGGGC
>c13lagging (SEQ ID NO: 159)
GCGCTGTCGCTGCCCA
>c13lagging (SEQ ID NO: 160)
GGCGACCGCCCTCTCG
>c13lagging (SEQ ID NO: 161)
TAGGGCGGTTCCCCTG
>c13lagging (SEQ ID NO: 162)
ACGATCTCGGGGGCTG
>c13lagging (SEQ ID NO: 163)
GGAAGCGGGGGGAGAA
>c13lagging (SEQ ID NO: 164)
CACCTCGCCCAGGGGT
>c13lagging (SEQ ID NO: 165)
CGGGTTGAAAGGTATG
>c13lagging (SEQ ID NO: 166)
TCTAAGTGGCATTTGA
>c13lagging (SEQ ID NO: 167)
CGGACGAGCAGGACGT
>c13lagging (SEQ ID NO: 168)
GTCTGCCTGTTTTCTA
>c13lagging (SEQ ID NO: 169)
CATATGGGCGTCCGGC
>c13lagging (SEQ ID NO: 170)
GCCAACGAGCGCCTCC
>c13lagging (SEQ ID NO: 171)
CCCAGTCGCCCAGCGG
>c13lagging (SEQ ID NO: 172)
GCGGTCGCCAGTCACG
>c13lagging (SEQ ID NO: 173)
CCCCCGGGGATACCTG
>c13lagging (SEQ ID NO: 174)
GCAGCGTGGCGCGCTC
>c13lagging (SEQ ID NO: 175)
TCCCTCGCGTCCGCCT
>c13lagging (SEQ ID NO: 176)
ACCCCGTCCCTGCATC
>c13lagging (SEQ ID NO: 177)
CCGGGTCAAGCTGACC
>c13lagging (SEQ ID NO: 178)
GACGAGCCTCCCAGGC
>c13lagging (SEQ ID NO: 179)
TAGCCGCGAGGGGCCG
>c13lagging (SEQ ID NO: 180)
TACCGCTATCCCAGTC
>c13lagging (SEQ ID NO: 181)
ACTCCTCCCGCTGGTA
>c13lagging (SEQ ID NO: 182)
GCCGGACGCCGGAGAG
>c13lagging (SEQ ID NO: 183)
CGCCTTTGCCTGCAGG
>c13lagging (SEQ ID NO: 184)
CAGCGGGAAACGCTCA
>c13lagging (SEQ ID NO: 185)
GCCGCTACCGCCCGGA
>c13lagging (SEQ ID NO: 186)
GTCGGACATGGCCCCA
>c13lagging (SEQ ID NO: 187)
GACTACATCGCGCGCC
>c13lagging (SEQ ID NO: 188)
CCGTGGAATGGGCCCC
>c13lagging (SEQ ID NO: 189)
GTAGGGGGACGTTGCA
>c13lagging (SEQ ID NO: 190)
TGCCGATTGACCTACT
>c13lagging (SEQ ID NO: 191)
CGCGCGCGCCTGTGCC
>c13lagging (SEQ ID NO: 192)
GAACTAGCGCGGCGGG
>c13lagging (SEQ ID NO: 193)
CTGTACGACGGCCACG
>c13lagging (SEQ ID NO: 194)
TTCGGATTCCTCTGCT
>c13lagging (SEQ ID NO: 195)
GCCCGGGCAACCCGAC
>c13lagging (SEQ ID NO: 196)
CGGTGGCCGGACAGGA
>c13lagging (SEQ ID NO: 197)
CGTAGGGAGCTCGGGG
>c13lagging (SEQ ID NO: 198)
CTTTCCCGGGCTGTCC
>c13lagging (SEQ ID NO: 199)
ACCCTGTCCCTCCCAG
>c13lagging (SEQ ID NO: 200)
AGCGGGGGGGCCGACT
>c13lagging (SEQ ID NO: 201)
GGATGCGGAACGACAG
>c13lagging (SEQ ID NO: 202)
GCGGGGCCCGCAAGGC
>c14leading (SEQ ID NO: 203)
CACTTAGTGCTCGTC
>c14leading (SEQ ID NO: 204)
CCGTTGATCGCTGGT
>c14leading (SEQ ID NO: 205)
GCATCAGGGCCTTGAGC
>c14leading (SEQ ID NO: 206)
TGGCCTGTTGGTGGGC
>c14leading (SEQ ID NO: 207)
CTGCTAACGCCGCCCT
>c14leading (SEQ ID NO: 208)
CCTGTGGACGAAGTGGA
>c14leading (SEQ ID NO: 209)
ATGAGAGACGTGCGCCA
>c14leading (SEQ ID NO: 210)
TGTTATCTCCGCCAGCC
>c14leading (SEQ ID NO: 211)
GTCCATGCGTGACCGGG
>c14leading (SEQ ID NO: 212)
TCGGGTACGACCGGGCC
>c14leading (SEQ ID NO: 213)
GGAACGGACTCACAC
>c14leading (SEQ ID NO: 214)
CCATGGTGAGCAATACG
>c14leading (SEQ ID NO: 215)
TATCCACCGGGAGCT
>c14leading (SEQ ID NO: 216)
TAGTTTGGCCCCCTT
>c14leading (SEQ ID NO: 217)
GGGCCGACCCGGGGAG
>c14leading (SEQ ID NO: 218)
TCCGAACGCTCGGGACT
>c14leading (SEQ ID NO: 219)
ACTACTTCACGAGCAAG
>c14leading (SEQ ID NO: 220)
CATCTCCGGCGCCGCTG
>c14leading (SEQ ID NO: 221)
CCCGCGATCTGGCACGC
>c14leading (SEQ ID NO: 222)
CCCTTCCAGACTCCT
>c14leading (SEQ ID NO: 223)
GGGCAGCGACAGCGC
>c14leading (SEQ ID NO: 224)
CGAGAGGGCGGTCGC
>c14leading (SEQ ID NO: 225)
CCAGGGGAACCGCCC
>c14leading (SEQ ID NO: 226)
TACAGCCCCCGAGATCG
>c14leading (SEQ ID NO: 227)
TTTCTCCCCCCGCTTCC
>c14leading (SEQ ID NO: 228)
ACCCCTGGGCGAGGTGC
>c14leading (SEQ ID NO: 229)
ATACCTTTCAACCCGTC
>c14leading (SEQ ID NO: 230)
AAATGCCACTTAGAACG
>c14leading (SEQ ID NO: 231)
TCCTGCTCGTCCGTAGA
>c14leading (SEQ ID NO: 232)
AAACAGGCAGACGCCGG
>c14leading (SEQ ID NO: 233)
ACGCCCATATGGGAGG
>c14leading (SEQ ID NO: 234)
CGCTCGTTGGCCCGC
>c14leading (SEQ ID NO: 235)
TGGGCGACTGGGCGTGA
>c14leading (SEQ ID NO: 236)
CTGGCGACCGCCAGGTA
>c14leading (SEQ ID NO: 237)
TCCCCGGGGGGAGCGCG
>c14leading (SEQ ID NO: 238)
CCACGCTGCAGGCGGAC
>c14leading (SEQ ID NO: 239)
GCGAGGGAGATGCAGG
>c14leading (SEQ ID NO: 240)
GACGGGGTGGTCAGCTT
>c14leading (SEQ ID NO: 241)
GACCCGGGCCTGGGAGG
>c14leading (SEQ ID NO: 242)
CTCGTCCGGCCCCTCGC
>c14leading (SEQ ID NO: 243)
GGCTAGACTGGGATAGC
>c14leading (SEQ ID NO: 244)
GGTATACCAGCGGGAGG
>c14leading (SEQ ID NO: 245)
AGTCTCTCCGGCGTCCG
>c14leading (SEQ ID NO: 246)
GCCCTGCAGGCAAAGG
>c14leading (SEQ ID NO: 247)
CGTGAGCGTTTCCCG
>c14leading (SEQ ID NO: 248)
CTGTCCGGGCGGTAGCG
>c14leading (SEQ ID NO: 249)
GCTGGGGCCATGTCCGA
>c14leading (SEQ ID NO: 250)
CGGCGCGCGATGTAGTC
>c14leading (SEQ ID NO: 251)
GGGGCCCATTCCACGGT
>c14leading (SEQ ID NO: 252)
GCAACGTCCCCCTAC
>c14leading (SEQ ID NO: 253)
AGTAGGTCAATCGGCAG
>c14leading (SEQ ID NO: 254)
GCACAGGCGCGCGCGCC
>c14leading (SEQ ID NO: 255)
CGCCGCGCTAGTTCC
>c14leading (SEQ ID NO: 256)
GTGGCCGTCGTACAG
>c14leading (SEQ ID NO: 257)
AGCAGAGGAATCCGAA
>c14leading (SEQ ID NO: 258)
GTCGGGTTGCCCGGG
>c14leading (SEQ ID NO: 259)
CTCCTGTCCGGCCACCG
>c14leading (SEQ ID NO: 260)
CCCCGAGCTCCCTAC
>c14leading (SEQ ID NO: 261)
GGGACAGCCCGGGAAA
>c14leading (SEQ ID NO: 262)
GCTGGGAGGGACAGGG
>c14leading (SEQ ID NO: 263)
TAGTCGGCCCCCCCG
>c14leading (SEQ ID NO: 264)
CTCTGTCGTTCCGCA
>c14leading (SEQ ID NO: 265)
TCCGCCTTGCGGGCC
>c14lagging (SEQ ID NO: 266)
ACGGGACGAGCACTA
>c14lagging (SEQ ID NO: 267)
ATGCACCAGCGATCA
>c14lagging (SEQ ID NO: 268)
GCCAGCTCAAGGCCCTG
>c14lagging (SEQ ID NO: 269)
GCAGGCCCACCAACAG
>c14lagging (SEQ ID NO: 270)
CAGGAGGGCGGCGTTA
>c14lagging (SEQ ID NO: 271)
TCATTCCACTTCGTCCA
>c14lagging (SEQ ID NO: 272)
AACATGGCGCACGTCTC
>c14lagging (SEQ ID NO: 273)
GGACGGCTGGCGGAGAT
>c14lagging (SEQ ID NO: 274)
CCGACCCGGTCACGCAT
>c14lagging (SEQ ID NO: 275)
TTCCGGCCCGGTCGTAC
>c14lagging (SEQ ID NO: 276)
ATGGGTGTGAGTCCG
>c14lagging (SEQ ID NO: 277)
GATACGTATTGCTCACC
>c14lagging (SEQ ID NO: 278)
ACTAAGCTCCCGGTG
>c14lagging (SEQ ID NO: 279)
GCCCAAGGGGGCCAA
>c14lagging (SEQ ID NO: 280)
CGGACTCCCCGGGTCG
>c14lagging (SEQ ID NO: 281)
TAGTAGTCCCGAGCGTT
>c14lagging (SEQ ID NO: 282)
GATGCTTGCTCGTGAAG
>c14lagging (SEQ ID NO: 283)
CGGGCAGCGGCGCCGGA
>c14lagging (SEQ ID NO: 284)
AGGGGCGTGCCAGATCG
>c14lagging (SEQ ID NO: 285)
GCCCAGGAGTCTGGA
>c14lagging (SEQ ID NO: 286)
CTCGGCGCTGTCGCT
>c14lagging (SEQ ID NO: 287)
CTGGGCGACCGCCCT
>c14lagging (SEQ ID NO: 288)
TGTAGGGCGGTTCCC
>c14lagging (SEQ ID NO: 289)
GAAACGATCTCGGGGGC
>c14lagging (SEQ ID NO: 290)
GGGTGGAAGCGGGGGGA
>c14lagging (SEQ ID NO: 291)
GTATGCACCTCGCCCAG
>c14lagging (SEQ ID NO: 292)
ATTTGACGGGTTGAAAG
>c14lagging (SEQ ID NO: 293)
AGGACGTTCTAAGTGGC
>c14lagging (SEQ ID NO: 294)
GTTTTCTACGGACGAGC
>c14lagging (SEQ ID NO: 295)
GCGTCCGGCGTCTGCCT
>c14lagging (SEQ ID NO: 296)
AGCGCCTCCCATATGG
>c14lagging (SEQ ID NO: 297)
CCCAGCGGGCCAACG
>c14lagging (SEQ ID NO: 298)
CCAGTCACGCCCAGTCG
>c14lagging (SEQ ID NO: 299)
GGGATACCTGGCGGTCG
>c14lagging (SEQ ID NO: 300)
GTGGCGCGCTCCCCCCG
>c14lagging (SEQ ID NO: 301)
TCGCGTCCGCCTGCAGC
>c14lagging (SEQ ID NO: 302)
CGTCCCTGCATCTCCC
>c14lagging (SEQ ID NO: 303)
GGTCAAGCTGACCACCC
>c14lagging (SEQ ID NO: 304)
CGAGCCTCCCAGGCCCG
>c14lagging (SEQ ID NO: 305)
AGCCGCGAGGGGCCGGA
>c14lagging (SEQ ID NO: 306)
TACCGCTATCCCAGTCT
>c14lagging (SEQ ID NO: 307)
GACTCCTCCCGCTGGTA
>c14lagging (SEQ ID NO: 308)
GGGCCGGACGCCGGAGA
>c14lagging (SEQ ID NO: 309)
CACGCCTTTGCCTGCA
>c14lagging (SEQ ID NO: 310)
ACAGCGGGAAACGCT
>c14lagging (SEQ ID NO: 311)
CAGCCGCTACCGCCCGG
>c14lagging (SEQ ID NO: 312)
GCCGTCGGACATGGCCC
>c14lagging (SEQ ID NO: 313)
CCCCGACTACATCGCGC
>c14lagging (SEQ ID NO: 314)
TTGCACCGTGGAATGGG
>c14lagging (SEQ ID NO: 315)
TACTGTAGGGGGACG
>c14lagging (SEQ ID NO: 316)
GTGCCTGCCGATTGACC
>c14lagging (SEQ ID NO: 317)
GGCGGGCGCGCGCGCCT
>c14lagging (SEQ ID NO: 318)
CCACGGAACTAGCGC
>c14lagging (SEQ ID NO: 319)
TGCTCTGTACGACGG
>c14lagging (SEQ ID NO: 320)
CGACTTCGGATTCCTC
>c14lagging (SEQ ID NO: 321)
GGAGCCCGGGCAACC
>c14lagging (SEQ ID NO: 322)
GGGGCGGTGGCCGGACA
>c14lagging (SEQ ID NO: 323)
TCCCGTAGGGAGCTC
>c14lagging (SEQ ID NO: 324)
CAGCTTTCCCGGGCTG
>c14lagging (SEQ ID NO: 325)
ACTACCCTGTCCCTCC
>c14lagging (SEQ ID NO: 326)
AGAGCGGGGGGGCCG
>c14lagging (SEQ ID NO: 327)
CGGATGCGGAACGAC
>c14lagging (SEQ ID NO: 328)
GCGGGGCCCGCAAGG

What is claimed is:

1. A method of polymer synthesis, the method comprising:
inputting a desired polynucleotide sequence in an in silico graph structure comprising a plurality of branches, each of which specifies a linear order of nucleic acids or xeno nucleic acids;
wherein the in silico graph structure is programmed for selecting an optimal combination of branches and vertices that specify the desired sequence and for directing the assembly of the polynucleotide molecule with the desired sequence.

2. The method of claim 1, wherein the branches correspond to oligonucleotides or polynucleotides in separated compartments.

3. The method of claim 1, wherein the in silico graph structure is resident in a computer system comprising program instructions executable to cause the system to:
generate a plurality of trees, each tree representing an ordered combination of operations to produce the desired polynucleotide sequence;
select the tree that provides an optimal combination of operations; and
direct the assembly of the desired polynucleotide sequence using the selected tree.

4. The method of claim 3, wherein the tree comprises branches representing sequences connected by nodes that represent attachments to be made between the sequences.

5. The method of claim 3, wherein the selecting step comprises calculating a score for each tree, the score representing a measure of success that the tree will result in the molecule with the desired polynucleotide sequence.

6. The method of claim 5, wherein the measure of success is based on stored scores or probabilities of success in assembling partial constructs and/or stored probabilities of mis-assemblies.

7. The method of claim 1, wherein the selecting step comprises identifying subgroups each comprising fewer than about twenty oligos; generating, scoring, and selecting a branch for each subgroup; and combining optimal branches from the subgroups to form an assembly tree having an optimal production score.

8. The method of claim 1, further comprising the step of directing a fluid handling system to make the desired polynucleotide, wherein the fluid handling system directs assembly of the desired polynucleotide in a microfluidic apparatus.

9. The method of claim 1, wherein a software package searches the graph structure to identify a local maxima, thereby to select one or more optimal branches.

10. The method of claim 1, wherein the in silico graph structure executes a machine learning algorithm to select said optimal combination of branches.

11. A method of synthesizing a polymer, the method comprising:
receiving sequence information describing a desired polynucleotide;
generating, by a computer system, a plurality of trees, each tree giving an order of attachments among oligos to form the desired polynucleotide;
selecting one of the trees having an optimal production score; and
directing, by the computing system, a liquid handling system to make the desired polynucleotide by joining the oligos as given by the selected tree.

12. The method of claim 11, wherein leaves of the trees represent the oligos and nodes of the trees represent attachments between oligos.

13. The method of claim 11, wherein the selecting step includes a calculating a score for each tree, the score representing a probability that the nucleic acid will be successfully made using that tree.

14. The method of claim 11, further comprising scoring the tree using (i) stored probabilities of success in ligating overhangs of the oligos; (ii) stored estimates of risk of mis-ligations between unintended pairs of the oligos; or (iii) both.

15. The method of claim 11, wherein there are greater than 10\hat{ }30 trees that give an order of attachments among the oligos to form the nucleic acid, and the method comprises:
performing the generating and selecting steps for a first subset of the oligos to form an intermediate tree; and storing the intermediate tree for use as a leaf in selecting a final tree having the optimal production score.

16. The method of claim 11, further comprising identifying subgroups each comprising fewer than about twenty of the oligos; generating, scoring, and selecting sub-trees for each subgroup; and joining together one optimal scoring tree from each subgroup to yield the selected tree having the optimal production score.

17. The method of claim 16, wherein for each subgroup, a software package generates all possible trees; stores all of the trees in memory; applies a scoring matrix to each of the trees in memory to score all of the trees; and selects a best-scoring tree for the sub-group.

18. The method of claim 11, wherein the desired polynucleotide comprises one substantially entire expression vector or organismal genome.

19. The method of claim 11, wherein making the desired polynucleotide comprises executing a software package to transform the selected tree into instructions executed by a liquid handling system to transfer the oligos among reaction vessels, in the order given by the selected tree to make the desired polynucleotide.

20. The method of claim 11, wherein a first leaf is connected to a root of the selected tree by a first number of nodes and edges and a second leaf is connected to the root of the selected tree by a second number not equal to the first.
